(19) United States
(12) Patent Application Publication
(10) Pub. No.: US 2024/0260453 A1
(43) Pub. Date: Aug. 1, 2024

(54) Organic Light-Emitting Compound And Organic Light-Emitting Device Comprising Same

(71) Applicant: P&H TECH Co., Ltd, Gyeonggi-do (KR)

(72) Inventors: Seo-Yong HYUN, Gyeonggi-do (KR); Seok-Keun YOON, Gyeonggi-do (KR); Gwan-hee PARK, Gyeonggi-do (KR)

(73) Assignee: P&H TECH Co., Ltd, Gyeonggi-do (KR)

(21) Appl. No.: 18/558,728

(22) PCT Filed: May 11, 2022
(86) PCT No.: PCT/KR2022/006716

§ 371 Date: Nov. 2, 2023

(30) Foreign Application Priority Data
May 14, 2021 (KR) 10-2021-0062392

(51) Int. Cl. H10K 85/60; C07D 405/12; C07D 409/10; C09K 11/06; H10K 50/18

(52) U.S. Cl. CPC H10K 85/636; C07D 405/12; C07D 409/10; C09K 11/06; H10K 85/633; C09K 2211/1018; H10K 50/181; H10K 85/615; H10K 85/6572; H10K 85/6574; H10K 85/6576

(57) ABSTRACT

An organic compound according to the present invention has, as the skeleton thereof, a structure that has a phenyl group incorporated at the —N terminus at position 9 in carbazole wherein an aryl(heteroaryl)amine group is incorporated via a linker at position 4 in carbazole, and the amine group includes a terphenyl group, dibenzofuran, or dibenzothiophene. Due to these structural characteristics, the compound according to the present invention may be employed in organic layers, such as an electron block layer, in organic light-emitting devices to realize an organic light-emitting device having light emission characteristics such as low driving voltage, excellent luminous efficiency, and the like of the device, and thus may be advantageously used industrially in a variety of display devices.

### TECHNICAL FIELD

The present invention relates to an organic light emitting compound, and more particularly, to an organic light emitting compound which is employed as a material for an organic layer, such as an electron blocking layer in an organic light emitting device, and an organic light emitting device which employs the same, thus achieving greatly improved luminescent properties such as low-voltage driving of the device and excellent luminous efficiency.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and read, and thus has recently become a subject of intense interest as a next-generation display device.

However, in order for such an organic light emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light emitting device has not yet been sufficiently made.

Thus, further improvements in terms of efficiency and life characteristics are required for good stability, high efficiency, long lifetime, and large size of organic light emitting devices. Particularly, there is a strong need to develop materials constituting each organic layer of organic light emitting devices.

### DISCLOSURE

### Technical Problem

Accordingly, an aspect of the present invention intends to provide a novel organic light emitting compound which is employed in an organic layer such as an electron blocking layer in an organic light emitting device, achieving excellent luminescent properties such as low-voltage driving of the device and improved luminous efficiency, and an organic light emitting device including the same.

### Technical Solution

An aspect of the present invention provides an organic light emitting compound represented by Formula I below and an organic light emitting device including the same in an organic layer such as an electron blocking layer in the device.

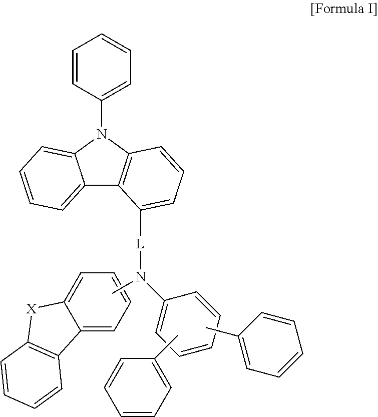

Characteristic structures of Formula I above and compounds L and X implemented thereby will be described later.

### Advantageous Effects

When an organic light emitting compound according to an embodiment of the present invention is employed as a material for an organic layer such as an electron blocking layer, in the organic light emitting device, it is possible to realize light emission characteristics such as low-voltage driving and excellent luminous efficiency of the device, thus can be usefully used in a variety of display devices.

### BEST MODE

Hereinafter, the present invention will be described in more detail.

The present invention relates to an organic light emitting device represented by Formula I that is employed for an organic layer, such as an electron blocking layer, in an organic light emitting device, achieving luminescent properties such as low-voltage driving of the device and excellent luminous efficiency.

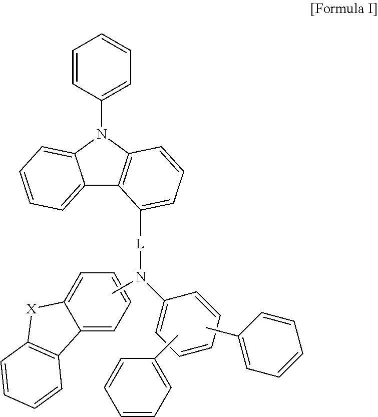

In Formula I, X is O or S and L is a divalent linking group of any one selected from groups represented by Structural Formula I below.

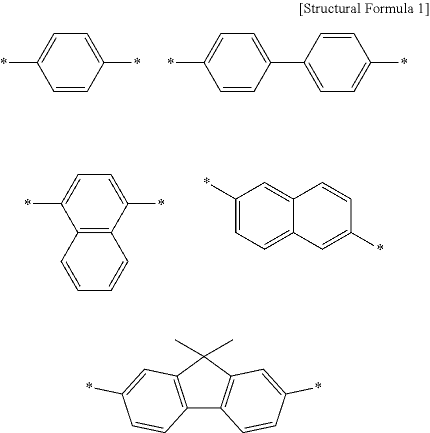

The compound according to an embodiment of the present invention is structurally characterized in that (i) a structure in which a phenyl group is introduced into a —N terminus at position 9 of carbazole is used as a skeleton as represented by Formula I (ii) an aryl (heteroaryl) amine group is introduced at position 4 of carbazole through phenylene, biphenylene, naphthylene, or fluorenylene linking group as represented by Structural Formula I, and (iii) the amine group consists of a terphenyl group, dibenzofuran, or dibenzothiophene.

The organic light emitting compound according to an embodiment of the present invention represented by Formula I above is used as an organic layer, such as an electron blocking layer, in an organic light emitting device, due to its structural features, thus improving low-voltage driving characteristic of the device and a luminous efficiency characteristic.

Preferred and specific examples of the organic light emitting compound represented by Formula I according to an embodiment of the present invention include the following compounds, but are not limited thereto.

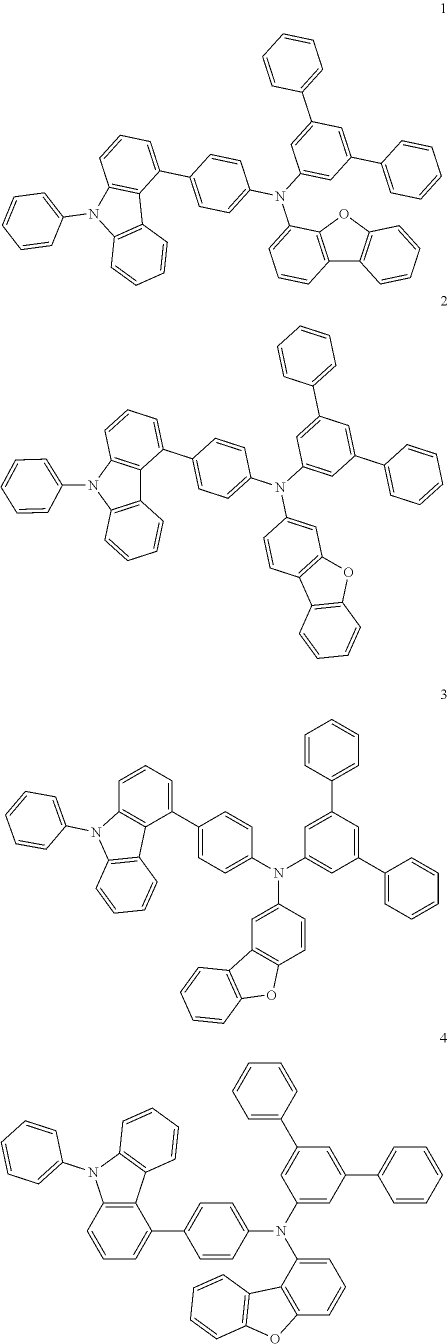

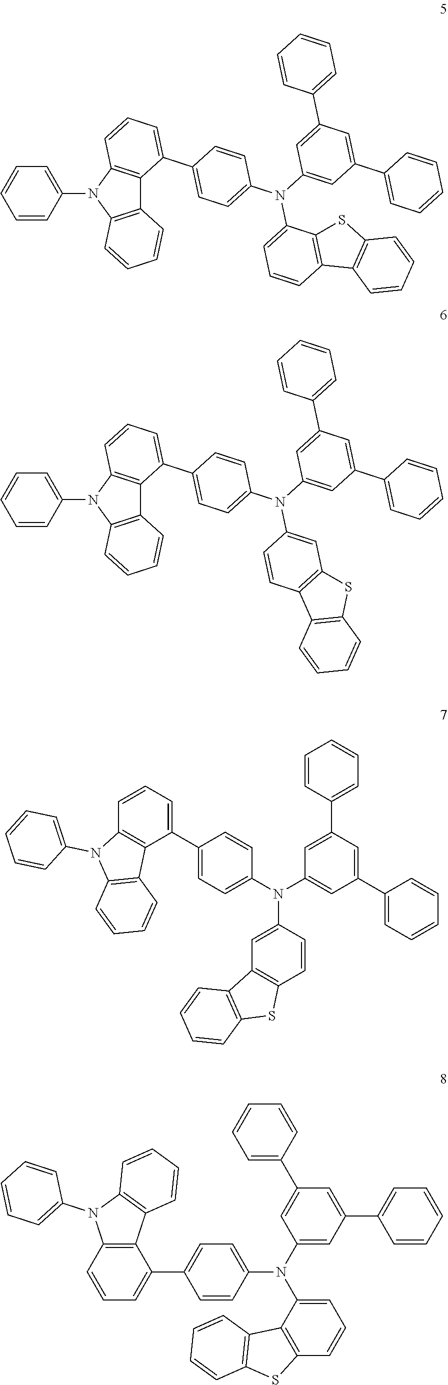 [SMILES: c1ccc(-c2cc(-c3ccccc3)cc(N(c3ccc(-c4cccc5c4c4ccccc4n5-c4ccccc4)cc3)c3cccc4c3sc3ccccc34)c2)cc1 ; c1ccc(-c2cc(-c3ccccc3)cc(N(c3ccc(-c4cccc5c4c4ccccc4n5-c4ccccc4)cc3)c3ccc4c(c3)sc3ccccc34)c2)cc1 ; c1ccc(-c2cc(-c3ccccc3)cc(N(c3ccc(-c4cccc5c4c4ccccc4n5-c4ccccc4)cc3)c3ccc4sc5ccccc5c4c3)c2)cc1 ; c1ccc(-c2cc(-c3ccccc3)cc(N(c3ccc(-c4cccc5c4c4ccccc4n5-c4ccccc4)cc3)c3cccc4sc5ccccc5c34)c2)cc1]

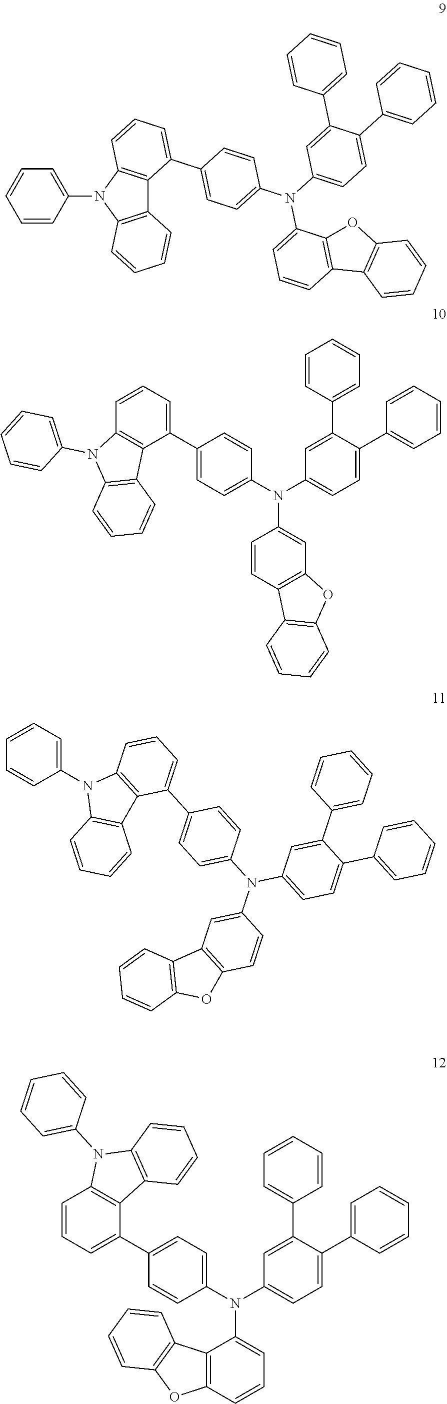 [SMILES: c1ccc(-c2ccc(N(c3ccc(-c4cccc5c4c4ccccc4n5-c4ccccc4)cc3)c3cccc4c3oc3ccccc34)cc2-c2ccccc2)cc1 ; c1ccc(-c2ccc(N(c3ccc(-c4cccc5c4c4ccccc4n5-c4ccccc4)cc3)c3ccc4c(c3)oc3ccccc34)cc2-c2ccccc2)cc1 ; c1ccc(-c2ccc(N(c3ccc(-c4cccc5c4c4ccccc4n5-c4ccccc4)cc3)c3ccc4oc5ccccc5c4c3)cc2-c2ccccc2)cc1 ; c1ccc(-c2ccc(N(c3ccc(-c4cccc5c4c4ccccc4n5-c4ccccc4)cc3)c3cccc4oc5ccccc5c34)cc2-c2ccccc2)cc1]

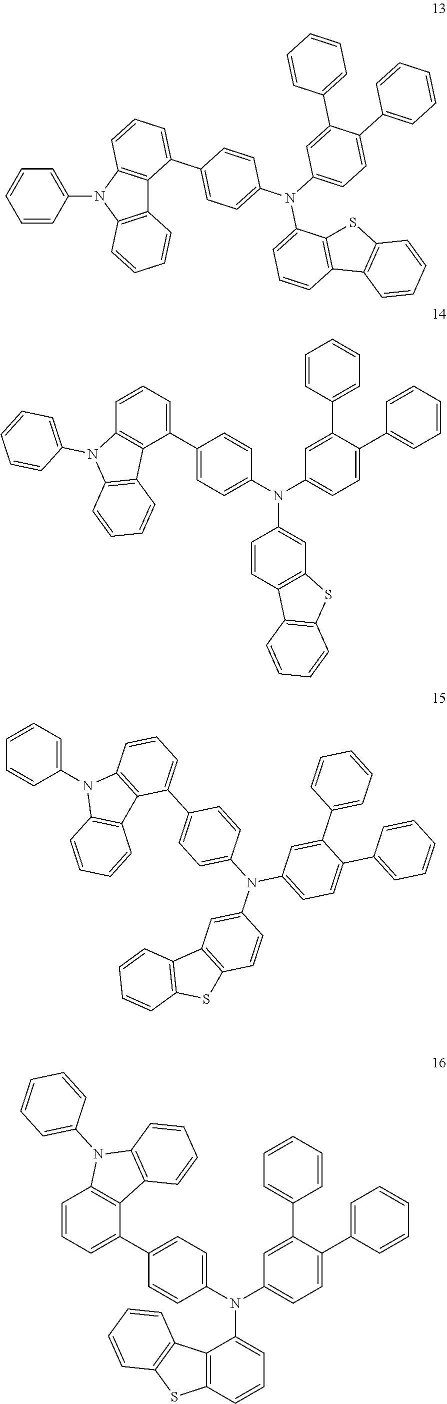 [SMILES: c1ccc(-c2ccc(N(c3ccc(-c4cccc5c4c4ccccc4n5-c4ccccc4)cc3)c3cccc4c3sc3ccccc34)cc2-c2ccccc2)cc1 ; c1ccc(-c2ccc(N(c3ccc(-c4cccc5c4c4ccccc4n5-c4ccccc4)cc3)c3ccc4c(c3)sc3ccccc34)cc2-c2ccccc2)cc1 ; c1ccc(-c2ccc(N(c3ccc(-c4cccc5c4c4ccccc4n5-c4ccccc4)cc3)c3ccc4sc5ccccc5c4c3)cc2-c2ccccc2)cc1 ; c1ccc(-c2ccc(N(c3ccc(-c4cccc5c4c4ccccc4n5-c4ccccc4)cc3)c3cccc4sc5ccccc5c34)cc2-c2ccccc2)cc1]

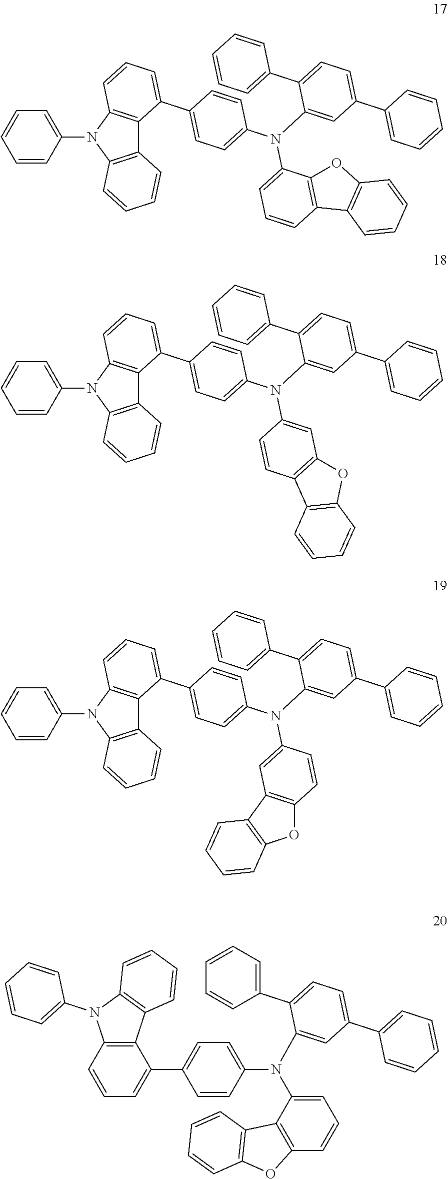 [SMILES: c1ccc(-c2ccc(-c3ccccc3)c(N(c3ccc(-c4cccc5c4c4ccccc4n5-c4ccccc4)cc3)c3cccc4c3oc3ccccc34)c2)cc1 ; c1ccc(-c2ccc(-c3ccccc3)c(N(c3ccc(-c4cccc5c4c4ccccc4n5-c4ccccc4)cc3)c3ccc4c(c3)oc3ccccc34)c2)cc1 ; c1ccc(-c2ccc(-c3ccccc3)c(N(c3ccc(-c4cccc5c4c4ccccc4n5-c4ccccc4)cc3)c3ccc4oc5ccccc5c4c3)c2)cc1 ; c1ccc(-c2ccc(-c3ccccc3)c(N(c3ccc(-c4cccc5c4c4ccccc4n5-c4ccccc4)cc3)c3cccc4oc5ccccc5c34)c2)cc1]

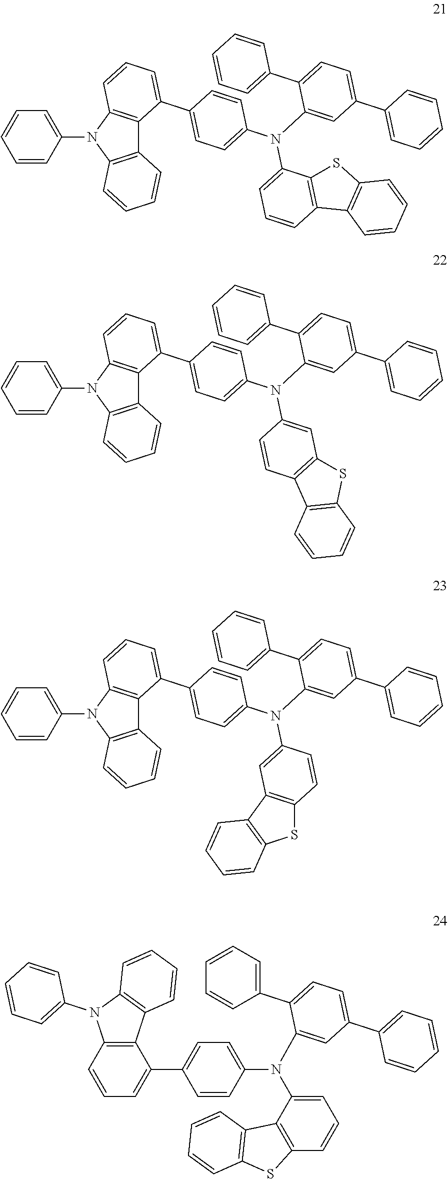 [SMILES: c1ccc(-c2ccc(-c3ccccc3)c(N(c3ccc(-c4cccc5c4c4ccccc4n5-c4ccccc4)cc3)c3cccc4c3sc3ccccc34)c2)cc1 ; c1ccc(-c2ccc(-c3ccccc3)c(N(c3ccc(-c4cccc5c4c4ccccc4n5-c4ccccc4)cc3)c3ccc4c(c3)sc3ccccc34)c2)cc1 ; c1ccc(-c2ccc(-c3ccccc3)c(N(c3ccc(-c4cccc5c4c4ccccc4n5-c4ccccc4)cc3)c3ccc4sc5ccccc5c4c3)c2)cc1 ; c1ccc(-c2ccc(-c3ccccc3)c(N(c3ccc(-c4cccc5c4c4ccccc4n5-c4ccccc4)cc3)c3cccc4sc5ccccc5c34)c2)cc1]

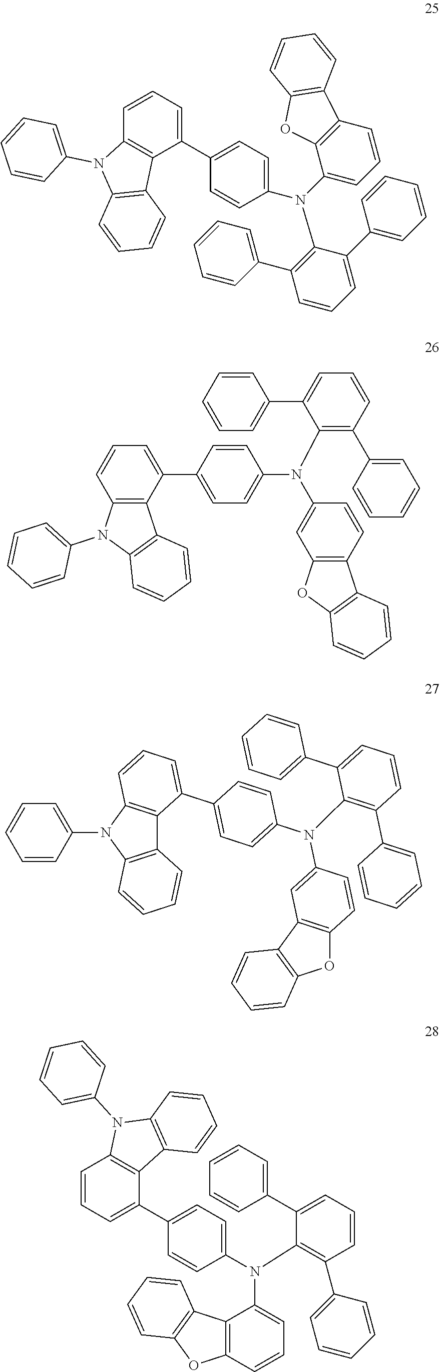 [SMILES: c1ccc(-c2cccc(-c3ccccc3)c2N(c2ccc(-c3cccc4c3c3ccccc3n4-c3ccccc3)cc2)c2cccc3c2oc2ccccc23)cc1 ; c1ccc(-c2cccc(-c3ccccc3)c2N(c2ccc(-c3cccc4c3c3ccccc3n4-c3ccccc3)cc2)c2ccc3c(c2)oc2ccccc23)cc1 ; c1ccc(-c2cccc(-c3ccccc3)c2N(c2ccc(-c3cccc4c3c3ccccc3n4-c3ccccc3)cc2)c2ccc3oc4ccccc4c3c2)cc1 ; c1ccc(-c2cccc(-c3ccccc3)c2N(c2ccc(-c3cccc4c3c3ccccc3n4-c3ccccc3)cc2)c2cccc3oc4ccccc4c23)cc1]

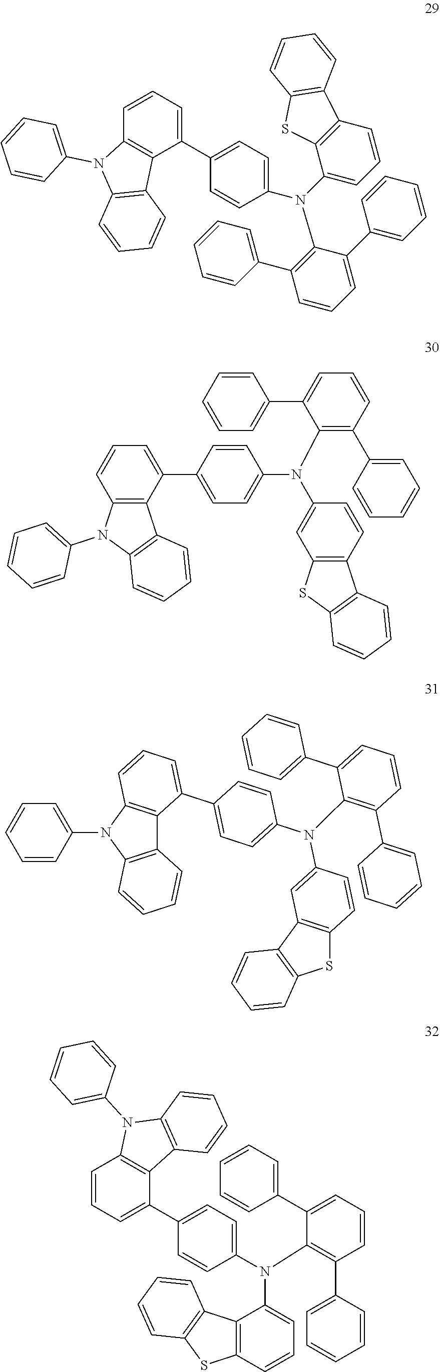 [SMILES: c1ccc(-c2cccc(-c3ccccc3)c2N(c2ccc(-c3cccc4c3c3ccccc3n4-c3ccccc3)cc2)c2cccc3c2sc2ccccc23)cc1 ; c1ccc(-c2cccc(-c3ccccc3)c2N(c2ccc(-c3cccc4c3c3ccccc3n4-c3ccccc3)cc2)c2ccc3c(c2)sc2ccccc23)cc1 ; c1ccc(-c2cccc(-c3ccccc3)c2N(c2ccc(-c3cccc4c3c3ccccc3n4-c3ccccc3)cc2)c2ccc3sc4ccccc4c3c2)cc1 ; c1ccc(-c2cccc(-c3ccccc3)c2N(c2ccc(-c3cccc4c3c3ccccc3n4-c3ccccc3)cc2)c2cccc3sc4ccccc4c23)cc1]

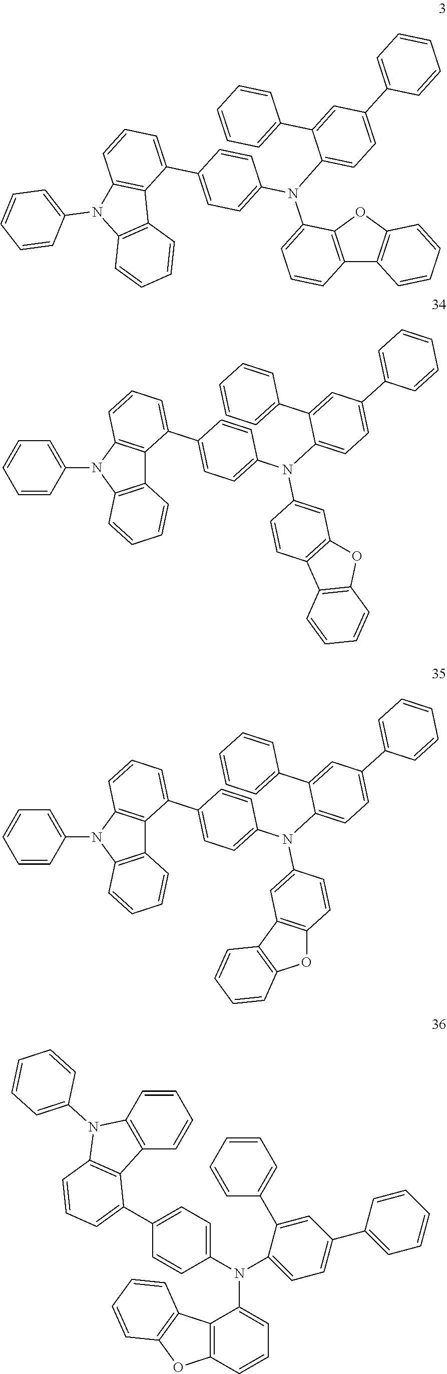

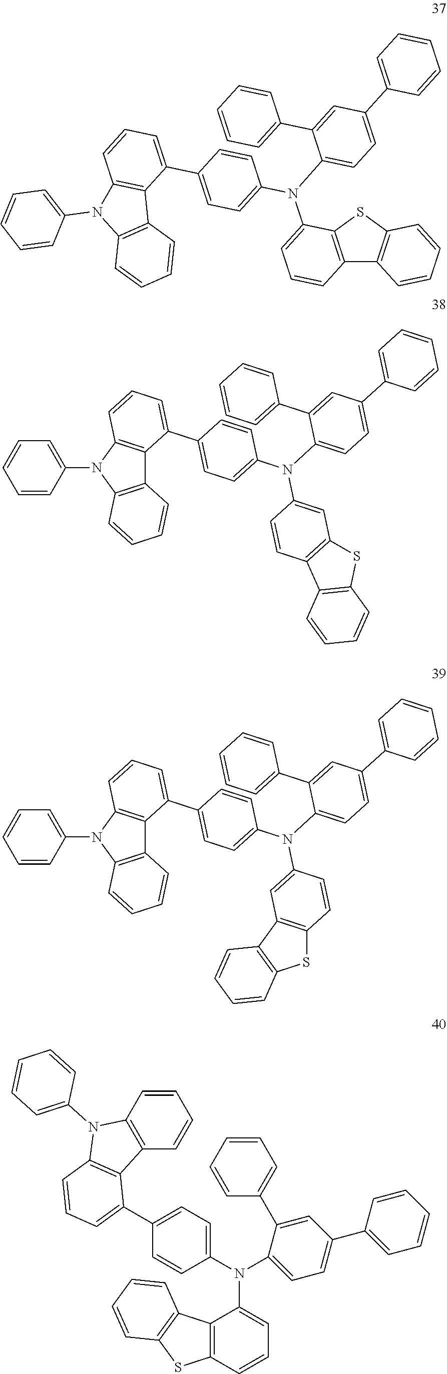

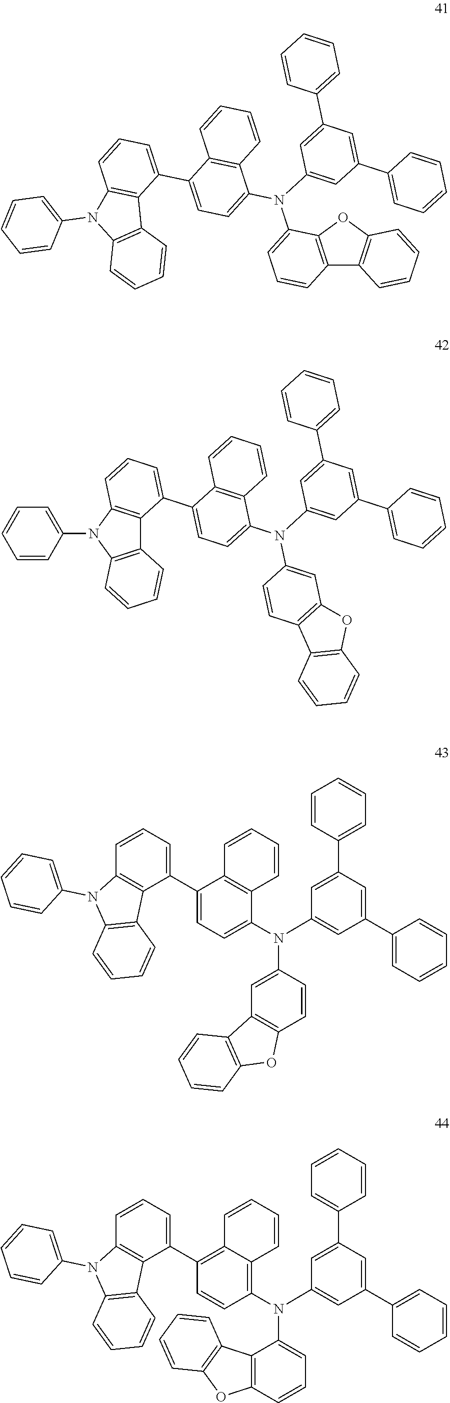 [SMILES: c1ccc(-c2cc(-c3ccccc3)cc(N(c3ccc(-c4cccc5c4c4ccccc4n5-c4ccccc4)c4ccccc34)c3cccc4c3oc3ccccc34)c2)cc1 ; c1ccc(-c2cc(-c3ccccc3)cc(N(c3ccc4c(c3)oc3ccccc34)c3ccc(-c4cccc5c4c4ccccc4n5-c4ccccc4)c4ccccc34)c2)cc1 ; c1ccc(-c2cc(-c3ccccc3)cc(N(c3ccc4oc5ccccc5c4c3)c3ccc(-c4cccc5c4c4ccccc4n5-c4ccccc4)c4ccccc34)c2)cc1 ; c1ccc(-c2cc(-c3ccccc3)cc(N(c3ccc(-c4cccc5c4c4ccccc4n5-c4ccccc4)c4ccccc34)c3cccc4oc5ccccc5c34)c2)cc1]

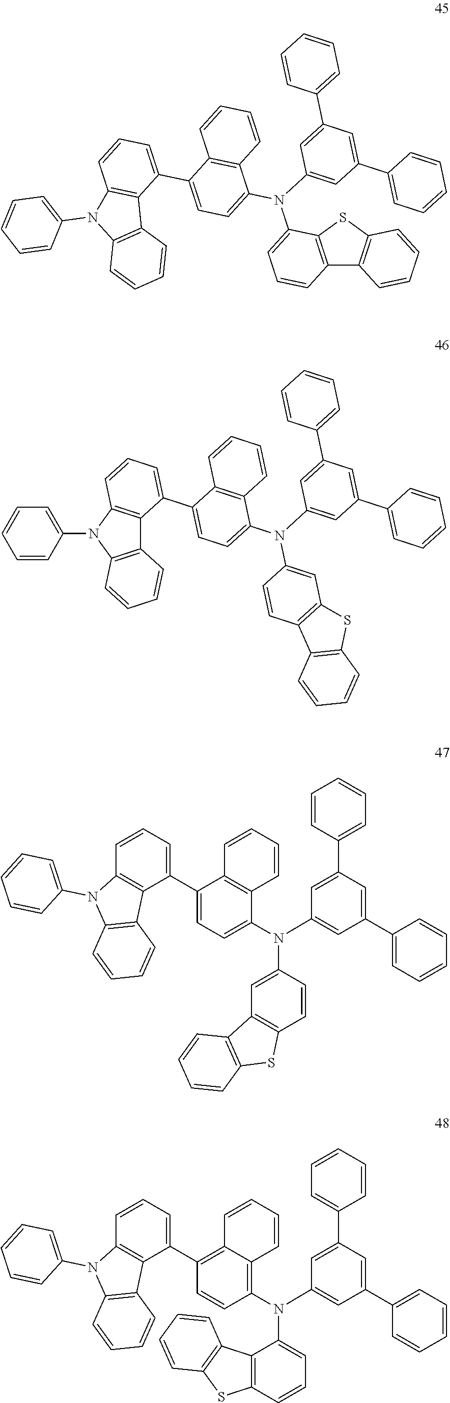 [SMILES: c1ccc(-c2cc(-c3ccccc3)cc(N(c3ccc(-c4cccc5c4c4ccccc4n5-c4ccccc4)c4ccccc34)c3cccc4c3sc3ccccc34)c2)cc1 ; c1ccc(-c2cc(-c3ccccc3)cc(N(c3ccc4c(c3)sc3ccccc34)c3ccc(-c4cccc5c4c4ccccc4n5-c4ccccc4)c4ccccc34)c2)cc1 ; c1ccc(-c2cc(-c3ccccc3)cc(N(c3ccc4sc5ccccc5c4c3)c3ccc(-c4cccc5c4c4ccccc4n5-c4ccccc4)c4ccccc34)c2)cc1 ; c1ccc(-c2cc(-c3ccccc3)cc(N(c3ccc(-c4cccc5c4c4ccccc4n5-c4ccccc4)c4ccccc34)c3cccc4sc5ccccc5c34)c2)cc1]

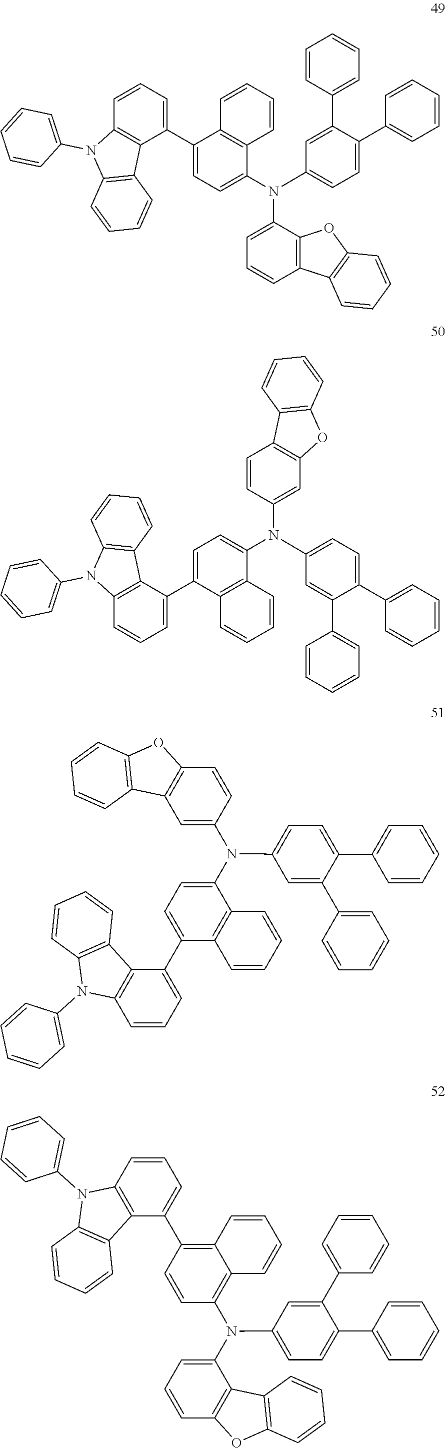 [SMILES: c1ccc(-c2ccc(N(c3ccc(-c4cccc5c4c4ccccc4n5-c4ccccc4)c4ccccc34)c3cccc4c3oc3ccccc34)cc2-c2ccccc2)cc1 ; c1ccc(-c2ccc(N(c3ccc4c(c3)oc3ccccc34)c3ccc(-c4cccc5c4c4ccccc4n5-c4ccccc4)c4ccccc34)cc2-c2ccccc2)cc1 ; c1ccc(-c2ccc(N(c3ccc4oc5ccccc5c4c3)c3ccc(-c4cccc5c4c4ccccc4n5-c4ccccc4)c4ccccc34)cc2-c2ccccc2)cc1 ; c1ccc(-c2ccc(N(c3ccc(-c4cccc5c4c4ccccc4n5-c4ccccc4)c4ccccc34)c3cccc4oc5ccccc5c34)cc2-c2ccccc2)cc1]

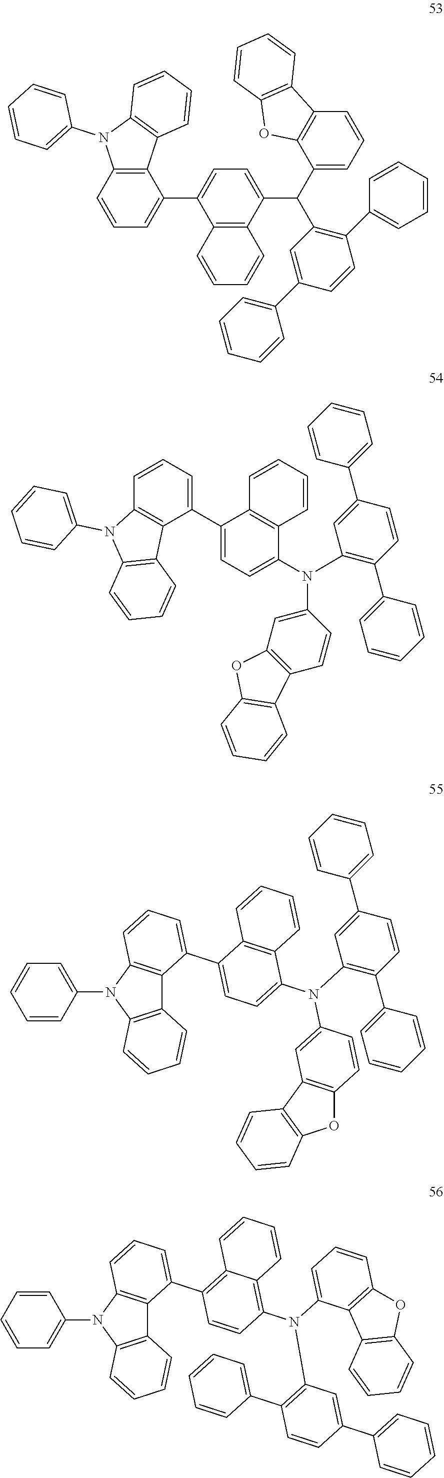 [SMILES: c1ccc(-c2ccc(-c3ccccc3)c(C(c3ccc(-c4cccc5c4c4ccccc4n5-c4ccccc4)c4ccccc34)c3cccc4c3oc3ccccc34)c2)cc1 ; c1ccc(-c2ccc(-c3ccccc3)c(N(c3ccc4c(c3)oc3ccccc34)c3ccc(-c4cccc5c4c4ccccc4n5-c4ccccc4)c4ccccc34)c2)cc1 ; c1ccc(-c2ccc(-c3ccccc3)c(N(c3ccc4oc5ccccc5c4c3)c3ccc(-c4cccc5c4c4ccccc4n5-c4ccccc4)c4ccccc34)c2)cc1 ; c1ccc(-c2ccc(-c3ccccc3)c(N(c3ccc(-c4cccc5c4c4ccccc4n5-c4ccccc4)c4ccccc34)c3cccc4oc5ccccc5c34)c2)cc1]

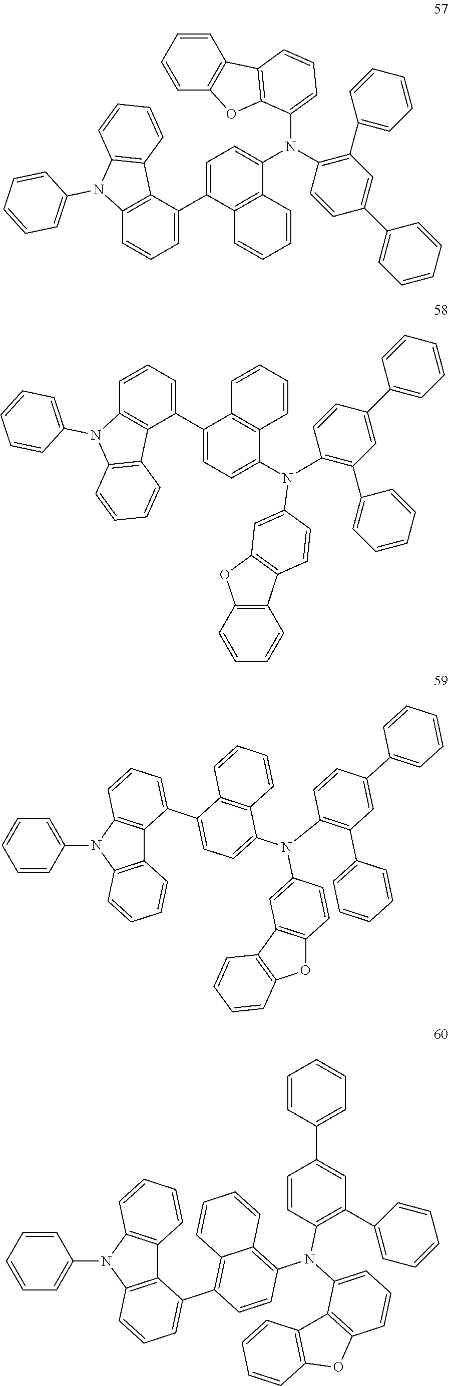 [SMILES: c1ccc(-c2ccc(N(c3ccc(-c4cccc5c4c4ccccc4n5-c4ccccc4)c4ccccc34)c3cccc4c3oc3ccccc34)c(-c3ccccc3)c2)cc1 ; c1ccc(-c2ccc(N(c3ccc4c(c3)oc3ccccc34)c3ccc(-c4cccc5c4c4ccccc4n5-c4ccccc4)c4ccccc34)c(-c3ccccc3)c2)cc1 ; c1ccc(-c2ccc(N(c3ccc4oc5ccccc5c4c3)c3ccc(-c4cccc5c4c4ccccc4n5-c4ccccc4)c4ccccc34)c(-c3ccccc3)c2)cc1 ; c1ccc(-c2ccc(N(c3ccc(-c4cccc5c4c4ccccc4n5-c4ccccc4)c4ccccc34)c3cccc4oc5ccccc5c34)c(-c3ccccc3)c2)cc1]

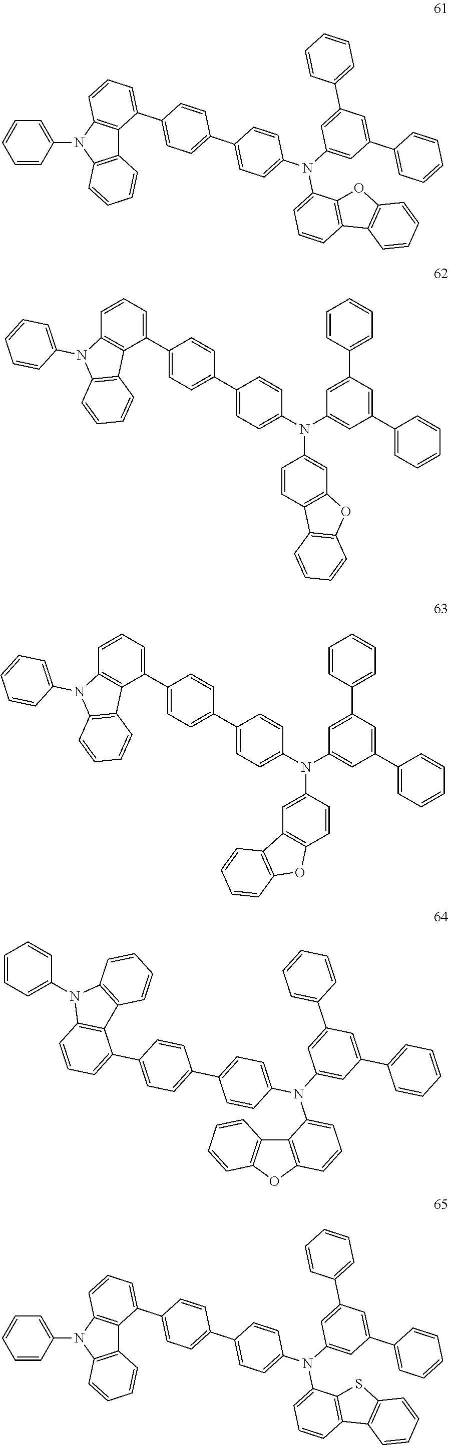

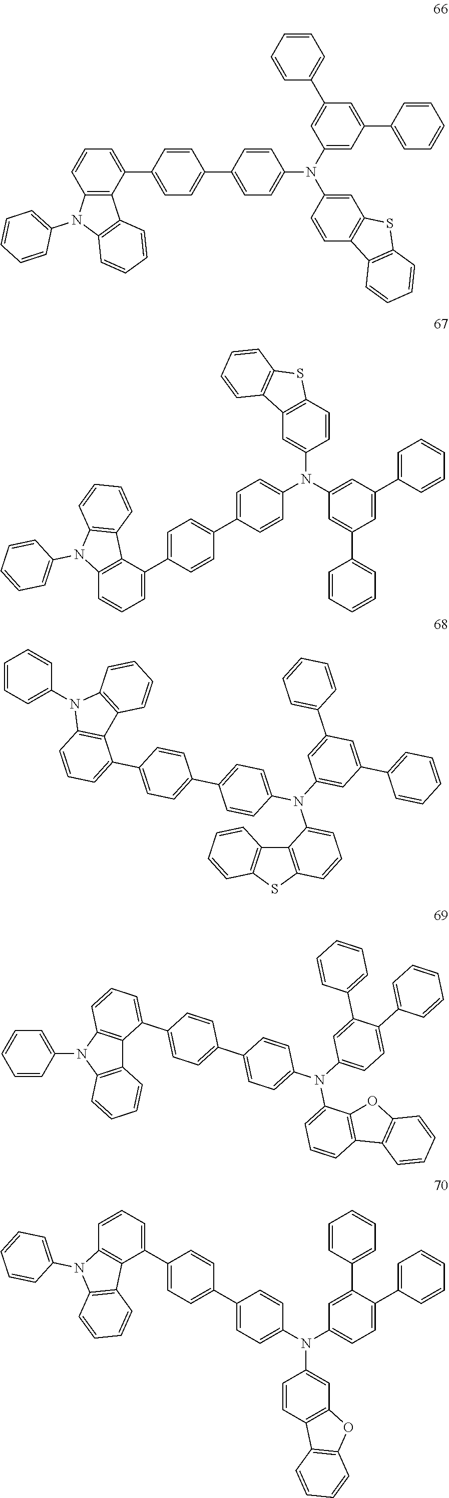

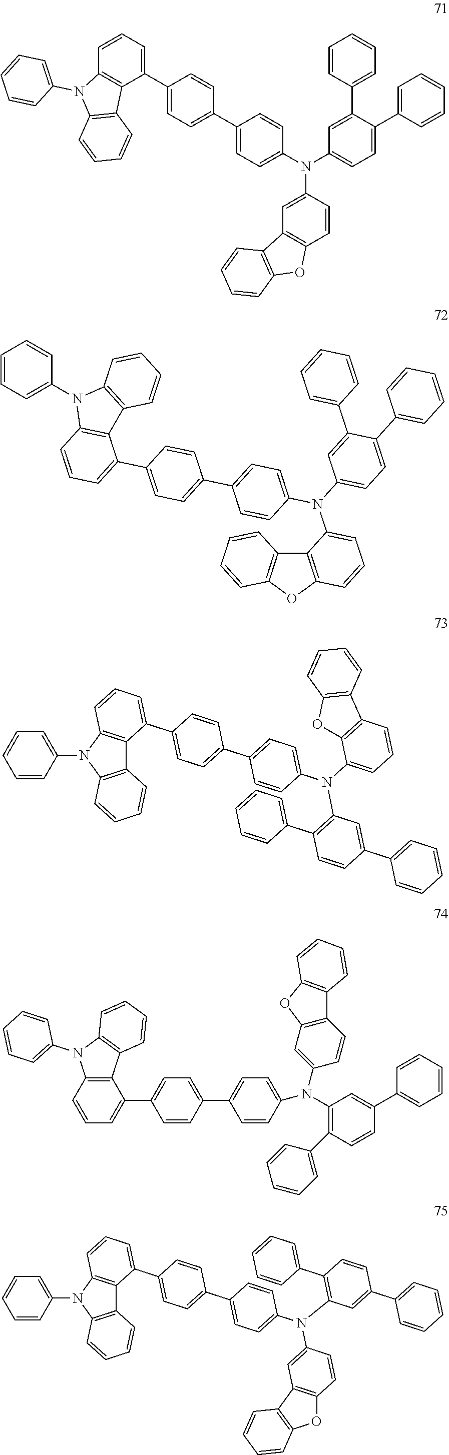

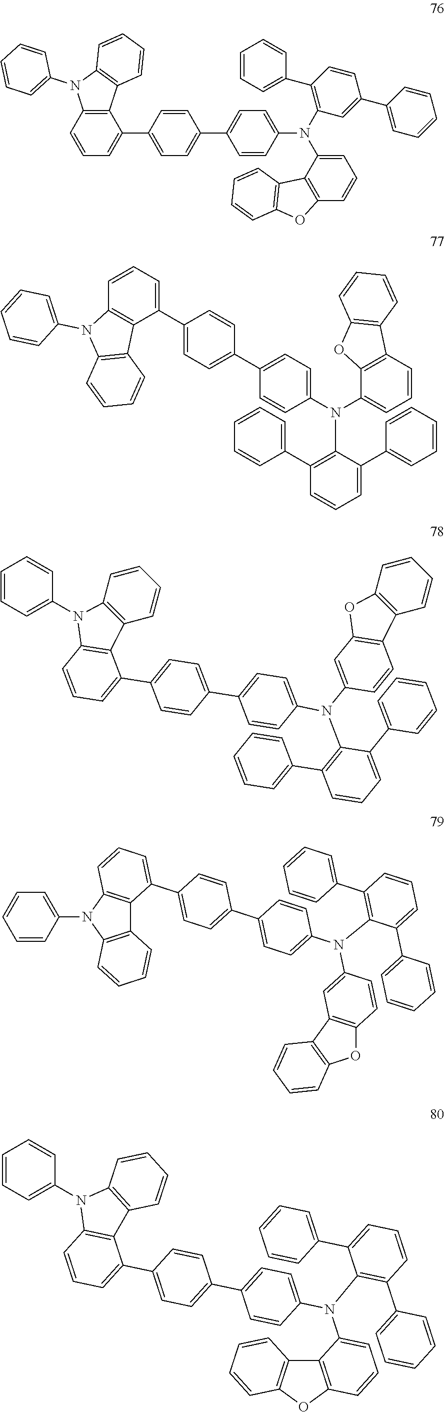

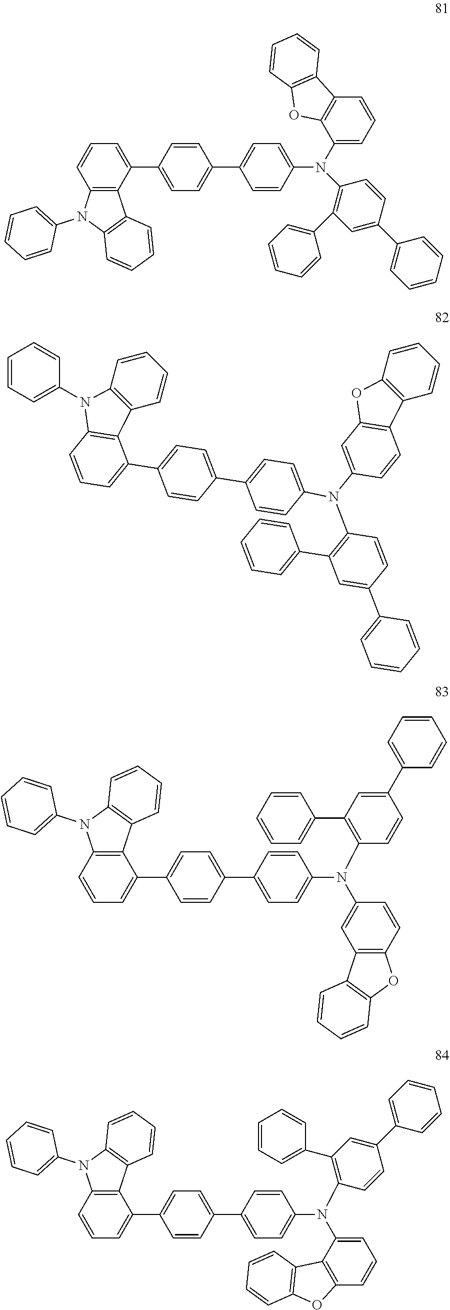 [SMILES: c1ccc(-c2ccc(N(c3ccc(-c4ccc(-c5cccc6c5c5ccccc5n6-c5ccccc5)cc4)cc3)c3cccc4c3oc3ccccc34)c(-c3ccccc3)c2)cc1 ; c1ccc(-c2ccc(N(c3ccc(-c4ccc(-c5cccc6c5c5ccccc5n6-c5ccccc5)cc4)cc3)c3ccc4c(c3)oc3ccccc34)c(-c3ccccc3)c2)cc1 ; c1ccc(-c2ccc(N(c3ccc(-c4ccc(-c5cccc6c5c5ccccc5n6-c5ccccc5)cc4)cc3)c3ccc4oc5ccccc5c4c3)c(-c3ccccc3)c2)cc1 ; c1ccc(-c2ccc(N(c3ccc(-c4ccc(-c5cccc6c5c5ccccc5n6-c5ccccc5)cc4)cc3)c3cccc4oc5ccccc5c34)c(-c3ccccc3)c2)cc1]

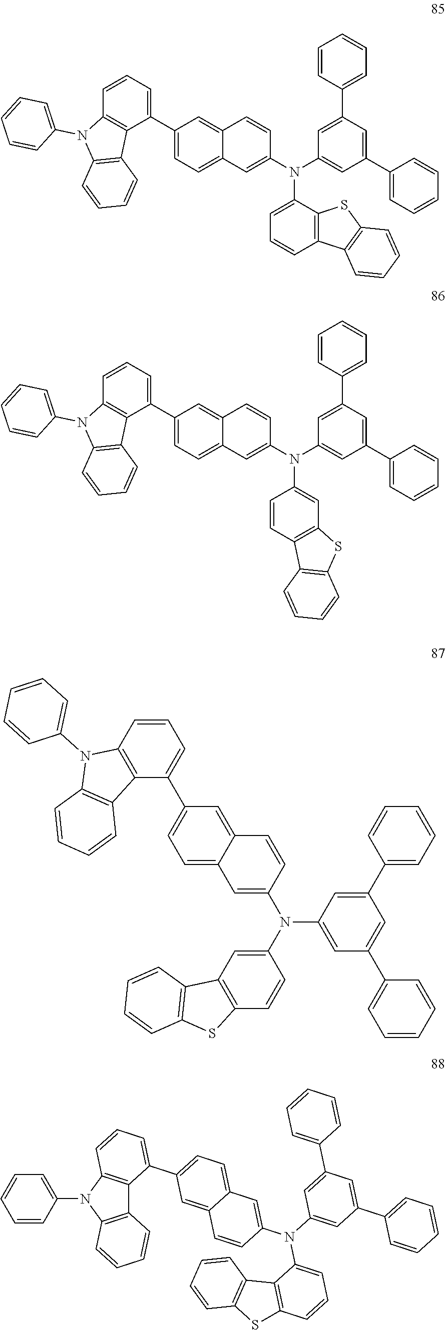 [SMILES: c1ccc(-c2cc(-c3ccccc3)cc(N(c3ccc4cc(-c5cccc6c5c5ccccc5n6-c5ccccc5)ccc4c3)c3cccc4c3sc3ccccc34)c2)cc1 ; c1ccc(-c2cc(-c3ccccc3)cc(N(c3ccc4cc(-c5cccc6c5c5ccccc5n6-c5ccccc5)ccc4c3)c3ccc4c(c3)sc3ccccc34)c2)cc1 ; c1ccc(-c2cc(-c3ccccc3)cc(N(c3ccc4cc(-c5cccc6c5c5ccccc5n6-c5ccccc5)ccc4c3)c3ccc4sc5ccccc5c4c3)c2)cc1 ; c1ccc(-c2cc(-c3ccccc3)cc(N(c3ccc4cc(-c5cccc6c5c5ccccc5n6-c5ccccc5)ccc4c3)c3cccc4sc5ccccc5c34)c2)cc1]

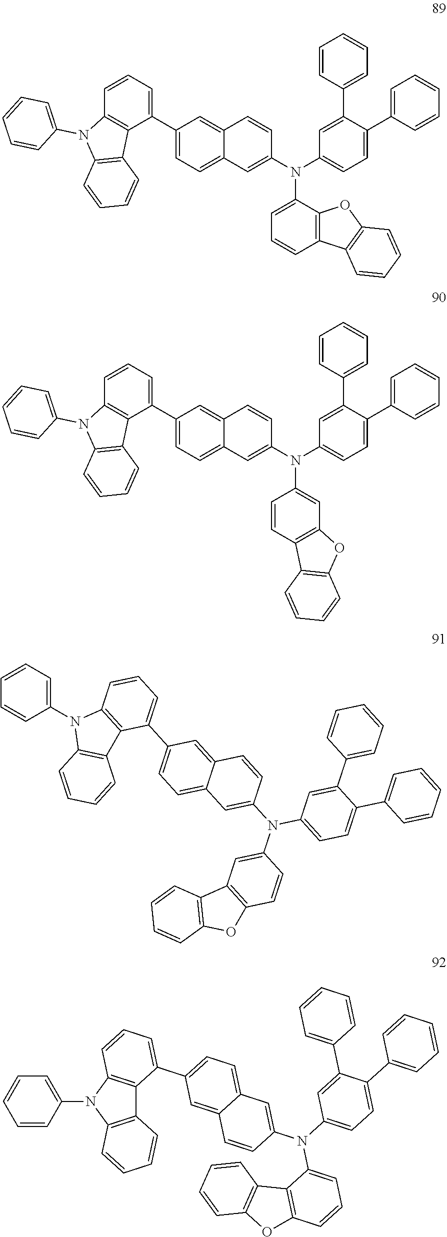 [SMILES: c1ccc(-c2ccc(N(c3ccc4cc(-c5cccc6c5c5ccccc5n6-c5ccccc5)ccc4c3)c3cccc4c3oc3ccccc34)cc2-c2ccccc2)cc1 ; c1ccc(-c2ccc(N(c3ccc4cc(-c5cccc6c5c5ccccc5n6-c5ccccc5)ccc4c3)c3ccc4c(c3)oc3ccccc34)cc2-c2ccccc2)cc1 ; c1ccc(-c2ccc(N(c3ccc4cc(-c5cccc6c5c5ccccc5n6-c5ccccc5)ccc4c3)c3ccc4oc5ccccc5c4c3)cc2-c2ccccc2)cc1 ; c1ccc(-c2ccc(N(c3ccc4cc(-c5cccc6c5c5ccccc5n6-c5ccccc5)ccc4c3)c3cccc4oc5ccccc5c34)cc2-c2ccccc2)cc1]

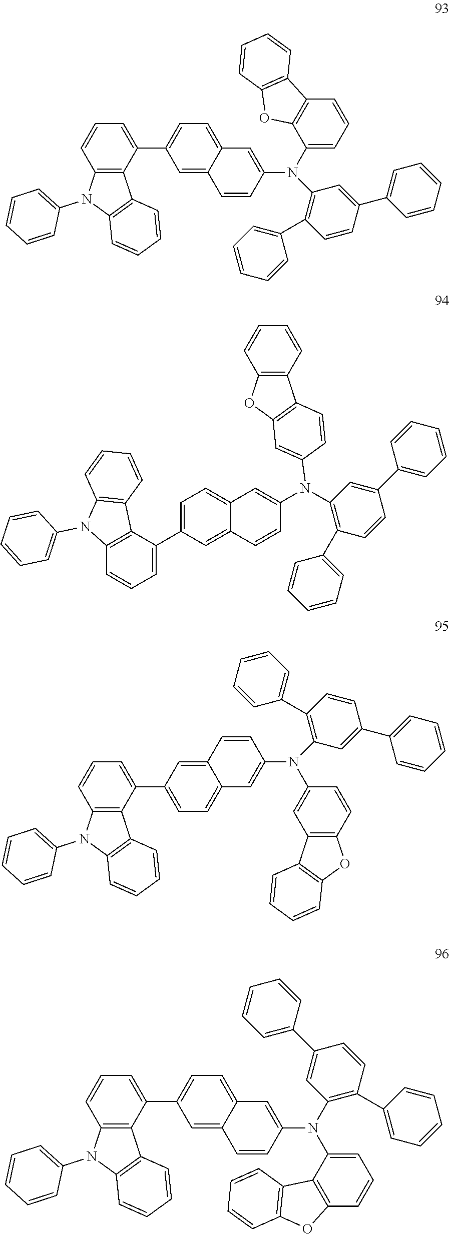 [SMILES: c1ccc(-c2ccc(-c3ccccc3)c(N(c3ccc4cc(-c5cccc6c5c5ccccc5n6-c5ccccc5)ccc4c3)c3cccc4c3oc3ccccc34)c2)cc1 ; c1ccc(-c2ccc(-c3ccccc3)c(N(c3ccc4cc(-c5cccc6c5c5ccccc5n6-c5ccccc5)ccc4c3)c3ccc4c(c3)oc3ccccc34)c2)cc1 ; c1ccc(-c2ccc(-c3ccccc3)c(N(c3ccc4cc(-c5cccc6c5c5ccccc5n6-c5ccccc5)ccc4c3)c3ccc4oc5ccccc5c4c3)c2)cc1 ; c1ccc(-c2ccc(-c3ccccc3)c(N(c3ccc4cc(-c5cccc6c5c5ccccc5n6-c5ccccc5)ccc4c3)c3cccc4oc5ccccc5c34)c2)cc1]

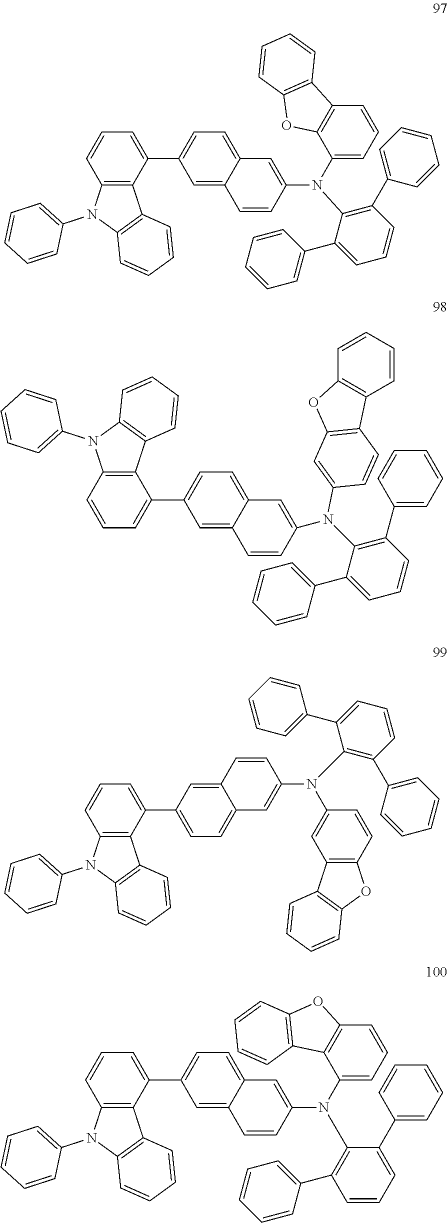 [SMILES: c1ccc(-c2cccc(-c3ccccc3)c2N(c2ccc3cc(-c4cccc5c4c4ccccc4n5-c4ccccc4)ccc3c2)c2cccc3c2oc2ccccc23)cc1 ; c1ccc(-c2cccc(-c3ccccc3)c2N(c2ccc3cc(-c4cccc5c4c4ccccc4n5-c4ccccc4)ccc3c2)c2ccc3c(c2)oc2ccccc23)cc1 ; c1ccc(-c2cccc(-c3ccccc3)c2N(c2ccc3cc(-c4cccc5c4c4ccccc4n5-c4ccccc4)ccc3c2)c2ccc3oc4ccccc4c3c2)cc1 ; c1ccc(-c2cccc(-c3ccccc3)c2N(c2ccc3cc(-c4cccc5c4c4ccccc4n5-c4ccccc4)ccc3c2)c2cccc3oc4ccccc4c23)cc1]

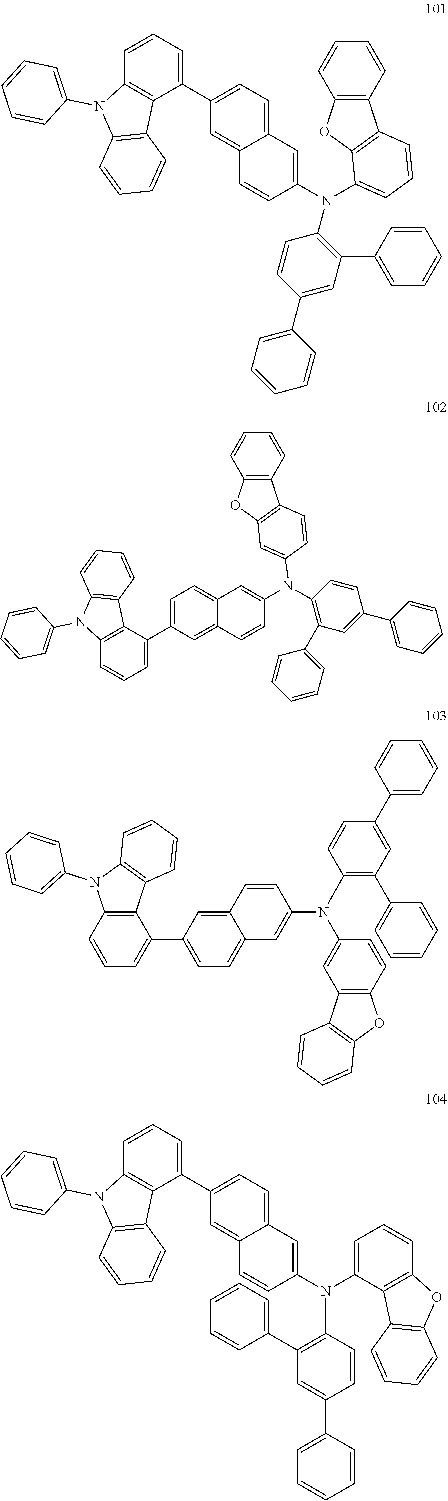

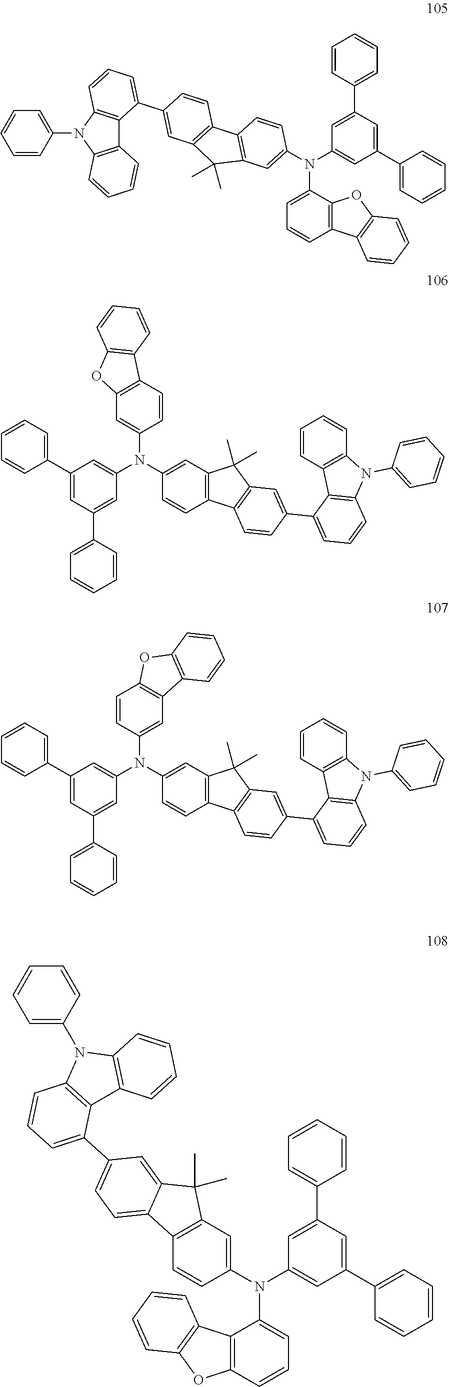 [SMILES: CC1(C)c2cc(-c3cccc4c3c3ccccc3n4-c3ccccc3)ccc2-c2ccc(N(c3cc(-c4ccccc4)cc(-c4ccccc4)c3)c3cccc4c3oc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4c(c3)oc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4oc5ccccc5c4c3)cc21 ; CC1(C)c2cc(-c3cccc4c3c3ccccc3n4-c3ccccc3)ccc2-c2ccc(N(c3cc(-c4ccccc4)cc(-c4ccccc4)c3)c3cccc4oc5ccccc5c34)cc21]

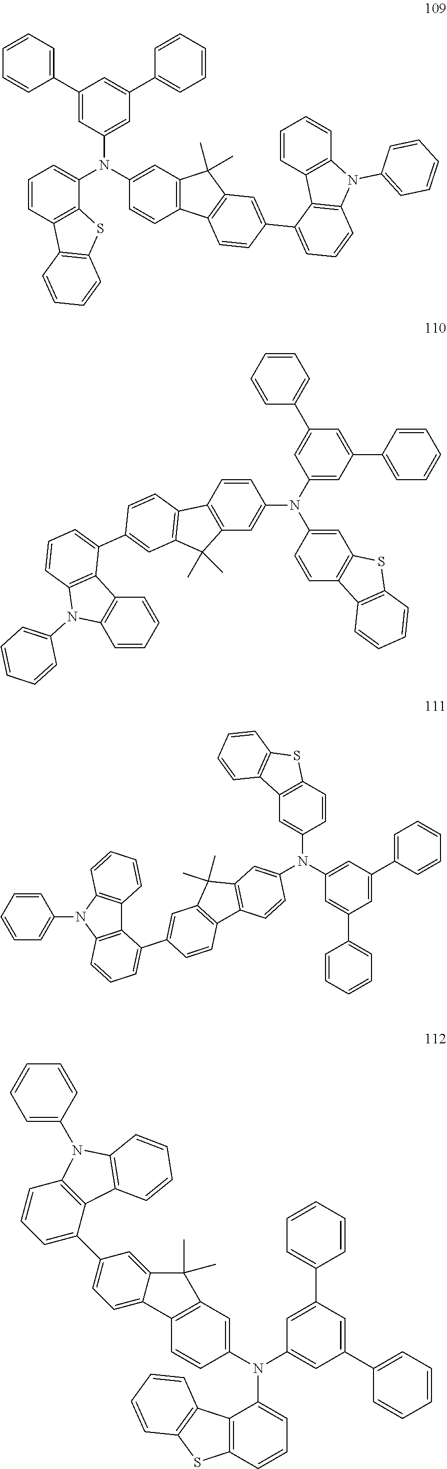 [SMILES: CC1(C)c2cc(-c3cccc4c3c3ccccc3n4-c3ccccc3)ccc2-c2ccc(N(c3cc(-c4ccccc4)cc(-c4ccccc4)c3)c3cccc4c3sc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4c(c3)sc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4sc5ccccc5c4c3)cc21 ; CC1(C)c2cc(-c3cccc4c3c3ccccc3n4-c3ccccc3)ccc2-c2ccc(N(c3cc(-c4ccccc4)cc(-c4ccccc4)c3)c3cccc4sc5ccccc5c34)cc21]

As such, the organic light emitting compound according to an embodiment of the present invention may synthesize an organic light emitting compound having various characteristics using a characteristic skeleton exhibiting inherent characteristics and moieties having inherent characteristics introduced into the skeleton. As a result, the organic light emitting compound according to an embodiment of the present invention may be employed as materials for various organic layers, such as a light emitting layer, a hole transport layer, an electron transport layer, an electron blocking layer, and a hole blocking layer, achieving further improved luminescent properties such as a luminous efficiency of the device by being used as a material of an electron blocking layer.

In addition, the compound of an embodiment of the present invention may be applied to a device according to a general method for manufacturing an organic light emitting device.

An organic light emitting device according to an embodiment of the present invention may include a first electrode, a second electrode, and an organic layer arranged therebetween. The organic light emitting device may be manufactured using a general device manufacturing method and material, except that the organic compound of an embodiment of the present invention is used to form the organic layer of the device.

The organic layer of the organic light emitting device according to an embodiment of the present invention may have a monolayer structure or a multilayer structure in which two or more organic layers are stacked. For example, the structure of the organic layers may include a hole injecting layer, a hole transport layer, a light emitting layer, an electron transport layer, an electron injecting layer, an electron blocking layer, a hole blocking layer, and a light efficiency improving layer (capping layer). The number of the organic layers is not limited and may be increased or decreased.

Preferred structures of the organic layers of the organic light emitting according to an embodiment of the present invention will be explained in more detail in the examples to be described later.

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light emitting device may be fabricated by depositing a cathode material, organic layer materials, and an anode material in this order on a substrate. The organic layers may have a multilayer structure including a hole injecting layer, a hole transport layer, a light emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene] (PEDT), polypyrrole, and polyaniline.

As the cathode material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport material is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light emitting device.

### Mode for Carrying Out the Disclosure

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 1

### (1) Preparative Example 1: Synthesis of Intermediate 1-1

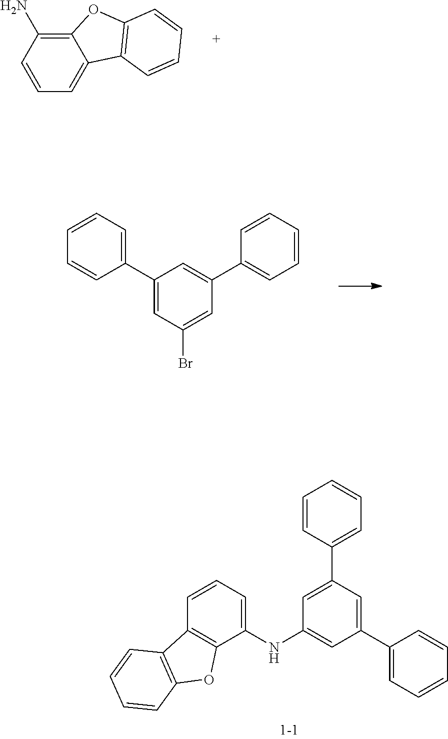 [SMILES: Nc1cccc2c1oc1ccccc12 ; Brc1cc(-c2ccccc2)cc(-c2ccccc2)c1 ; c1ccc(-c2cc(Nc3cccc4c3oc3ccccc34)cc(-c3ccccc3)c2)cc1]

Toluene 150 ml was added to Dibenzo[b,d]furan-4-amine (10.0 g, 0.055 mol), 1-Bromo-3,5-diphenylbenzene (25.3 g, 0.082 mol), NaOtBu (10.5 g, 0.109 mol), Pd(dba)₂ (1.6 g, 0.003 mol), t-Bu3P (1.1 g, 0.006 mol), and then was reacted with stirring at 70° C. for 4 hours. After completion of the reaction, the reaction mixture was extracted, concentrated, and then subjected to column and recrystallization to obtain 13.8 g (yield 61.4%) of Intermediate 1-1.

### (2) Preparative Example 2: Synthesis of Compound 1

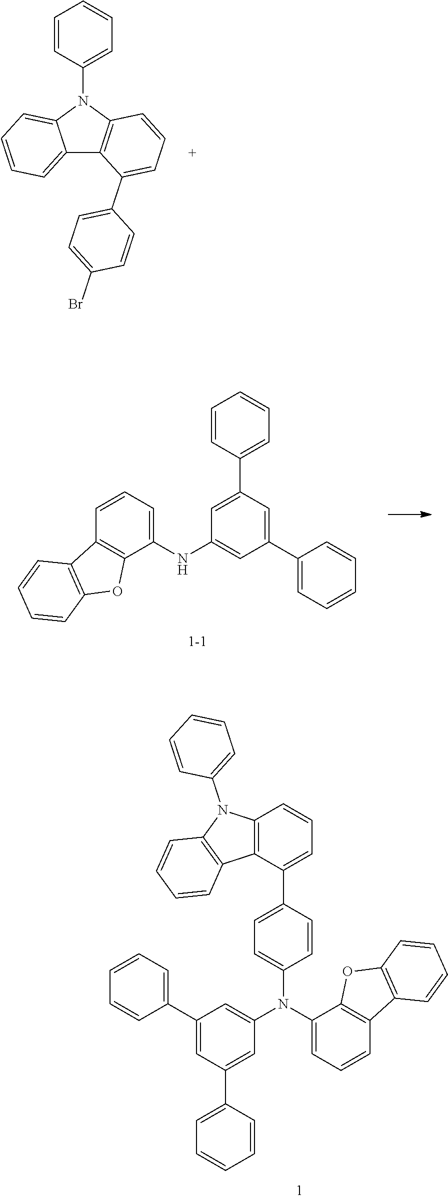 [SMILES: Brc1ccc(-c2cccc3c2c2ccccc2n3-c2ccccc2)cc1 ; c1ccc(-c2cc(Nc3cccc4c3oc3ccccc34)cc(-c3ccccc3)c2)cc1 ; c1ccc(-c2cc(-c3ccccc3)cc(N(c3ccc(-c4cccc5c4c4ccccc4n5-c4ccccc4)cc3)c3cccc4c3oc3ccccc34)c2)cc1]

Toluene 150 ml was added to 4-4-Bromophenyl-N-phenylcarbazole (10.0 g, 0.025 mol), Intermediate 1-1 (15.5 g, 0.038 mol), NaOtBu (4.8 g, 0.050 mol), Pd(dba)₂ (0.7 g, 0.003 mol), t-Bu3P (0.5 g, 0.006 mol), and then were reacted with stirring at 70° C. for 4 hours. After completion of the reaction, the reaction mixture was extracted, concentrated, and then subjected to column and recrystallization to obtain 11.8 g (yield 77.5%) of Compound 1.

LC/MS: m/z=728[(M)⁺]

### Synthesis Example 2: Synthesis of Compound 2

### (1) Preparative Example 1: Synthesis of Intermediate 2-1

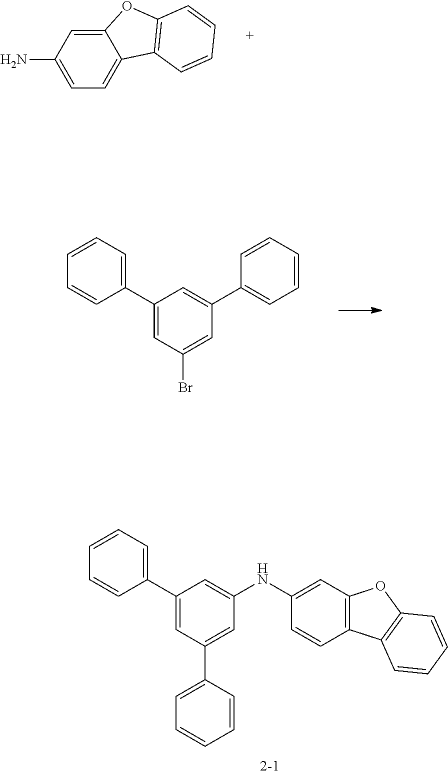 [SMILES: Nc1ccc2c(c1)oc1ccccc12 ; Brc1cc(-c2ccccc2)cc(-c2ccccc2)c1 ; c1ccc(-c2cc(Nc3ccc4c(c3)oc3ccccc34)cc(-c3ccccc3)c2)cc1]

Toluene 150 ml was added to Dibenzo[b,d]furan-3-amine (10.0 g, 0.072 mol), 1-Bromo-3,5-diphenylbenzene (33.6 g, 0.108 mol), NaOtBu (13.9 g, 0.145 mol), Pd(dba)₂ (2.1 g, 0.004 mol), t-Bu3P (1.5 g, 0.007 mol), and then was reacted with stirring at 70° C. for 4 hours. After completion of the reaction, the reaction mixture was extracted, concentrated, and then subjected to column and recrystallization to obtain 19.2 g (yield 64.5%) of Intermediate 2-1.

### (2) Preparative Example 2: Synthesis of Compound 2

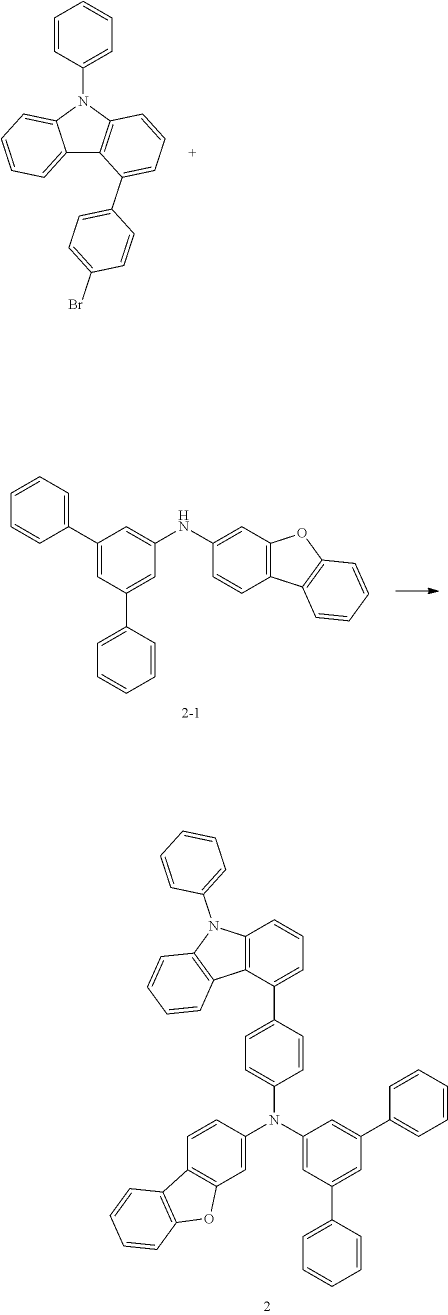 [SMILES: Brc1ccc(-c2cccc3c2c2ccccc2n3-c2ccccc2)cc1 ; c1ccc(-c2cc(Nc3ccc4c(c3)oc3ccccc34)cc(-c3ccccc3)c2)cc1 ; c1ccc(-c2cc(-c3ccccc3)cc(N(c3ccc(-c4cccc5c4c4ccccc4n5-c4ccccc4)cc3)c3ccc4c(c3)oc3ccccc34)c2)cc1]

Toluene 150 ml was added to 4-4-Bromophenyl-N-phenylcarbazole (10.0 g, 0.025 mol), Intermediate 2-1 (15.5 g, 0.038 mol), NaOtBu (4.8 g, 0.050 mol), Pd(dba)₂ (0.7 g, 0.003 mol), t-Bu3P (0.5 g, 0.006 mol), and then was reacted with stirring at 70° C. for 4 hours. After completion of the reaction, the reaction mixture was extracted, concentrated, and then subjected to column and recrystallization to obtain 13.3 g (yield 72.7%) of Compound 2.

LC/MS: m/z=728[(M)⁺]

### Synthesis Example 3: Synthesis of Compound 7

### (1) Preparative Example 1: Synthesis of Intermediate 7-1

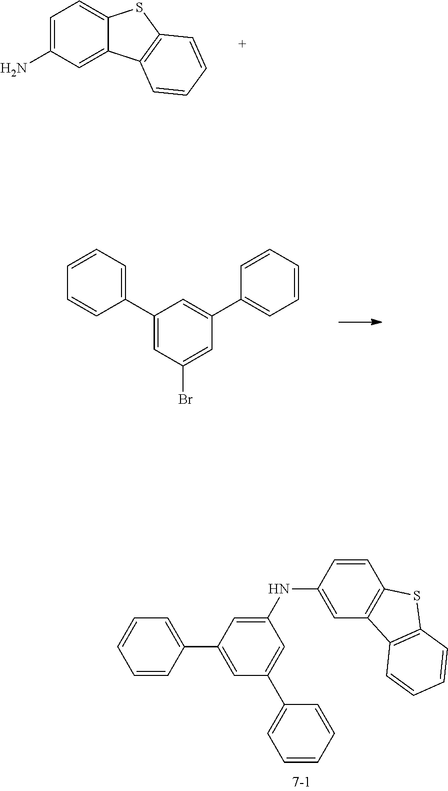 [SMILES: Nc1ccc2sc3ccccc3c2c1 ; Brc1cc(-c2ccccc2)cc(-c2ccccc2)c1 ; c1ccc(-c2cc(Nc3ccc4sc5ccccc5c4c3)cc(-c3ccccc3)c2)cc1]

Toluene 150 ml was added to 2-Aminodibenzothiophene (10.0 g, 0.050 mol), 1-Bromo-3,5-diphenylbenzene (23.3 g, 0.075 mol), NaOtBu (9.7 g, 0.100 mol), Pd(dba)₂ (1.4 g, 0.003 mol), t-Bu3P (1.0 g, 0.005 mol), and then was reacted with stirring at 70° C. for 4 hours. After completion of the reaction, the reaction mixture was extracted, concentrated, and then subjected to column and recrystallization to obtain 12.7 g (yield 59.2%) of Intermediate 7-1.

### (2) Preparative Example 2: Synthesis of Compound 7

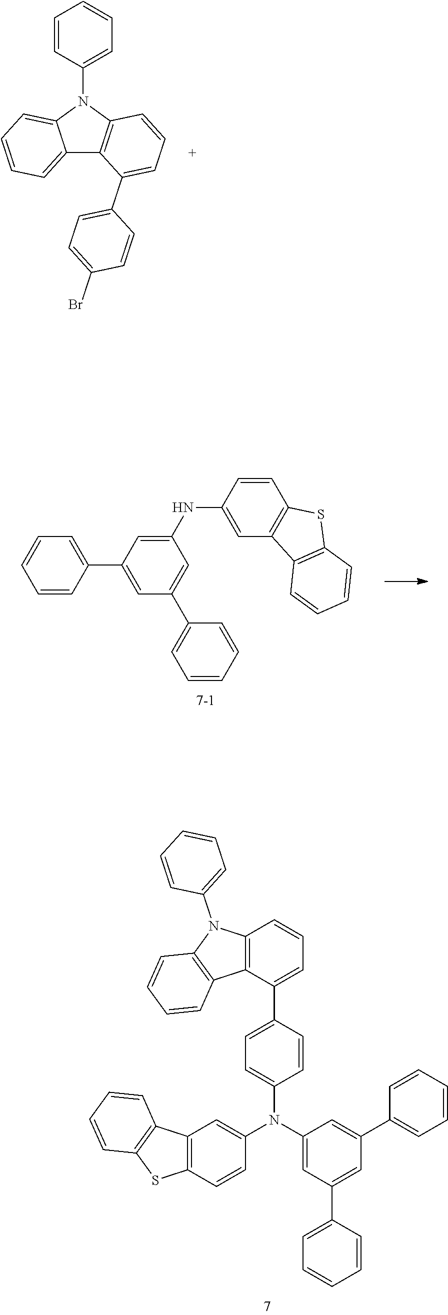 [SMILES: Brc1ccc(-c2cccc3c2c2ccccc2n3-c2ccccc2)cc1 ; c1ccc(-c2cc(Nc3ccc4sc5ccccc5c4c3)cc(-c3ccccc3)c2)cc1 ; c1ccc(-c2cc(-c3ccccc3)cc(N(c3ccc(-c4cccc5c4c4ccccc4n5-c4ccccc4)cc3)c3ccc4sc5ccccc5c4c3)c2)cc1]

Toluene 150 ml was added to 4-4-Bromophenyl-N-phenylcarbazole (10.0 g, 0.025 mol), Intermediate 7-1 (16.1 g, 0.038 mol), NaOtBu (4.8 g, 0.050 mol), Pd(dba)₂ (0.7 g, 0.003 mol), t-Bu₃P (0.5 g, 0.006 mol), and then was reacted with stirring at 70° C. for 4 hours. After completion of the reaction, the reaction mixture was extracted, concentrated, and then subjected to column and recrystallization to obtain 12.2 g (yield 65.2%) of Compound 7.

LC/MS: m/z=744[(M)⁺]

### Synthesis Example 4: Synthesis of Compound 12

### (1) Preparative Example 1: Synthesis of Intermediate 12-1

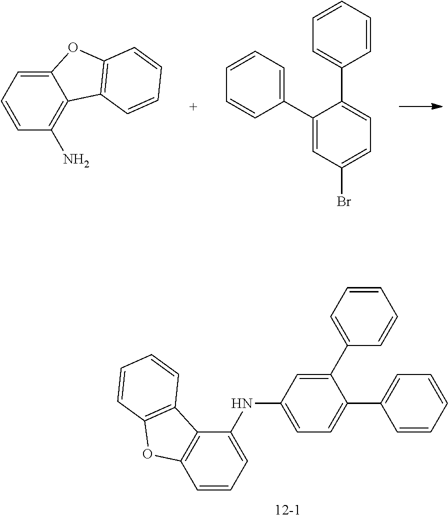 [SMILES: Nc1cccc2oc3ccccc3c12 ; Brc1ccc(-c2ccccc2)c(-c2ccccc2)c1 ; c1ccc(-c2ccc(Nc3cccc4oc5ccccc5c34)cc2-c2ccccc2)cc1]

Toluene 150 ml was added to Dibenzo[b,d]furan-1-amine (10.0 g, 0.055 mol), 1-Bromo-3.4-diphenylbenzene (25.3 g, 0.082 mol), NaOtBu (10.5 g, 0.109 mol), Pd(dba)₂ (1.6 g, 0.003 mol), t-Bu3P (1.1 g, 0.006 mol), and then was reacted with stirring at 70° C. for 4 hours. After completion of the reaction, the reaction mixture was extracted, concentrated, and then subjected to column and recrystallization to obtain 11.5 g (yield 51.2%) of Intermediate 12-1.

### (2) Preparative Example 2: Synthesis of Compound 12

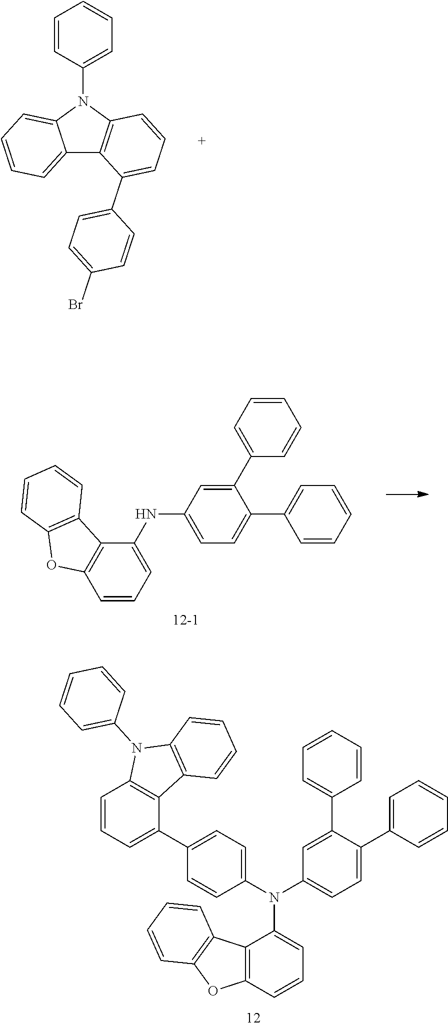 [SMILES: Brc1ccc(-c2cccc3c2c2ccccc2n3-c2ccccc2)cc1 ; c1ccc(-c2ccc(Nc3cccc4oc5ccccc5c34)cc2-c2ccccc2)cc1 ; c1ccc(-c2ccc(N(c3ccc(-c4cccc5c4c4ccccc4n5-c4ccccc4)cc3)c3cccc4oc5ccccc5c34)cc2-c2ccccc2)cc1]

Toluene 150 ml was added to 4-4-Bromophenyl-N-phenylcarbazole (10.0 g, 0.025 mol), Intermediate 12-1 (15.5 g, 0.038 mol), NaOtBu (4.8 g, 0.050 mol), Pd(dba)₂ (0.7 g, 0.003 mol), t-Bu₃P (0.5 g, 0.006 mol), and then was reacted with stirring at 70° C. for 4 hours. After completion of the reaction, the reaction mixture was extracted, concentrated, and then subjected to column and recrystallization to obtain 12.5 g (yield 68.3%) of Compound 12.

LC/MS: m/z=728[(M)⁺]

### Synthesis Example 5: Synthesis of Compound 18

### (1) Preparative Example 1: Synthesis of Intermediate 18-1

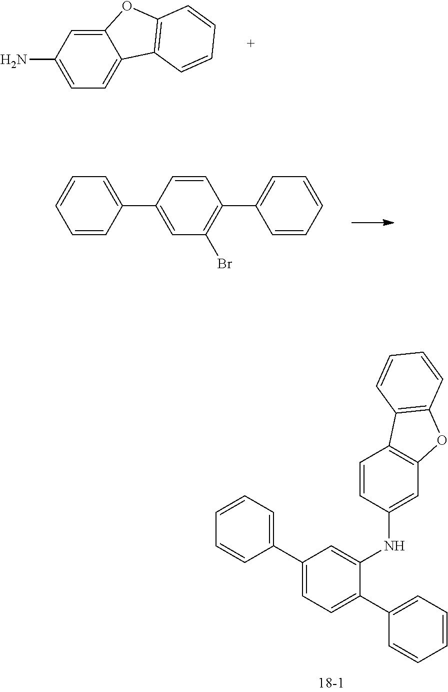 [SMILES: Nc1ccc2c(c1)oc1ccccc12 ; Brc1cc(-c2ccccc2)ccc1-c1ccccc1 ; c1ccc(-c2ccc(-c3ccccc3)c(Nc3ccc4c(c3)oc3ccccc34)c2)cc1]

Toluene 150 ml was added to Dibenzo[b,d]furan-3-amine (10.0 g, 0.055 mol), 2′-Bromo-1,l′:4′,1″-terphenyl (25.3 g, 0.082 mol), NaOtBu (10.5 g, 0.109 mol), Pd(dba)₂ (1.6 g, 0.003 mol), t-Bu₃P (1.1 g, 0.006 mol), and then was reacted with stirring at 70° C. for 4 hours. After completion of the reaction, the reaction mixture was extracted, concentrated, and then subjected to column and recrystallization to obtain 10.9 g (yield 48.5%) of Compound 18-1.

### (2) Preparative Example 2: Synthesis of Compound 18

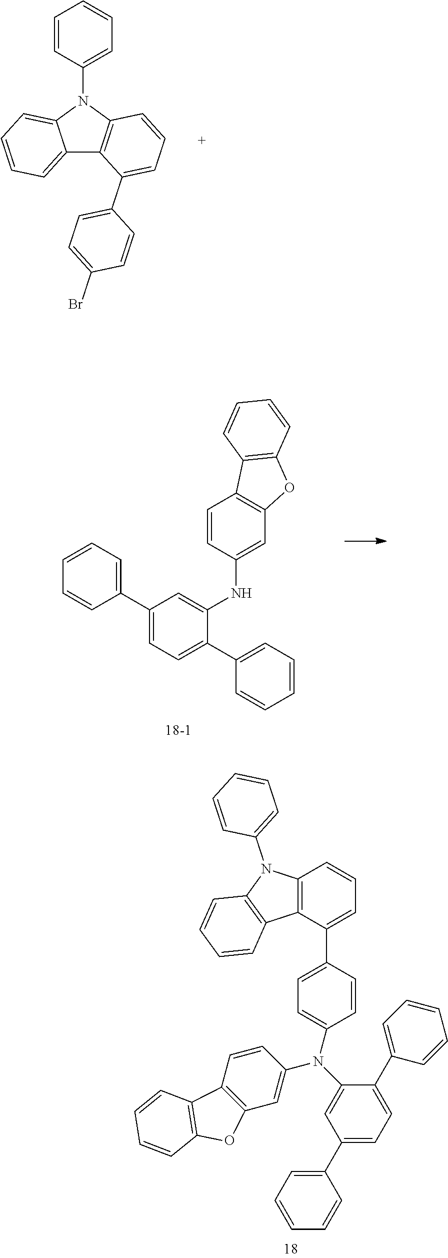 [SMILES: Brc1ccc(-c2cccc3c2c2ccccc2n3-c2ccccc2)cc1 ; c1ccc(-c2ccc(-c3ccccc3)c(Nc3ccc4c(c3)oc3ccccc34)c2)cc1 ; c1ccc(-c2ccc(-c3ccccc3)c(N(c3ccc(-c4cccc5c4c4ccccc4n5-c4ccccc4)cc3)c3ccc4c(c3)oc3ccccc34)c2)cc1]

Toluene 150 ml was added to 4-4-Bromophenyl-N-phenylcarbazole (10.0 g, 0.025 mol), Intermediate 18-1 (15.5 g, 0.038 mol), NaOtBu (4.8 g, 0.050 mol), Pd(dba)₂ (0.7 g, 0.003 mol), t-Bu₃P (0.5 g, 0.006 mol), and then was reacted with stirring at 70° C. for 4 hours. After completion of the reaction, the reaction mixture was extracted, concentrated, and then subjected to column and recrystallization to obtain 13.5 g (yield 73.8%) of Compound 18.

LC/MS: m/z=728[(M)⁺]

### Synthesis Example 6: Synthesis of Compound 29

### (1) Preparative Example 1: Synthesis of Intermediate 29-1

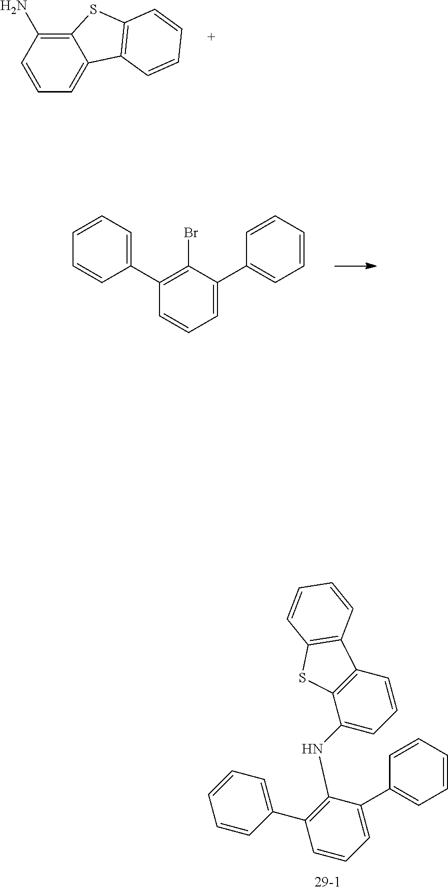 [SMILES: Nc1cccc2c1sc1ccccc12 ; Brc1c(-c2ccccc2)cccc1-c1ccccc1 ; c1ccc(-c2cccc(-c3ccccc3)c2Nc2cccc3c2sc2ccccc23)cc1]

Toluene 150 ml was added to Dibenzo[b,d]thiophen-4-amine (10.0 g, 0.050 mol), 2′-Bromo-1,1′:3′,1″-terphenyl (23.3 g, 0.075 mol), NaOtBu (9.7 g, 0.100 mol), Pd(dba)₂ (1.4 g, 0.003 mol), t-Bu₃P (1.0 g, 0.005 mol), and then was reacted with stirring at 70° C. for 4 hours. After completion of the reaction, the reaction mixture was extracted, concentrated, and then subjected to column and recrystallization to obtain 11.7 g (yield 54.5%) of Intermediate 29-1.

### (2) Preparative Example 2: Synthesis of Compound 29

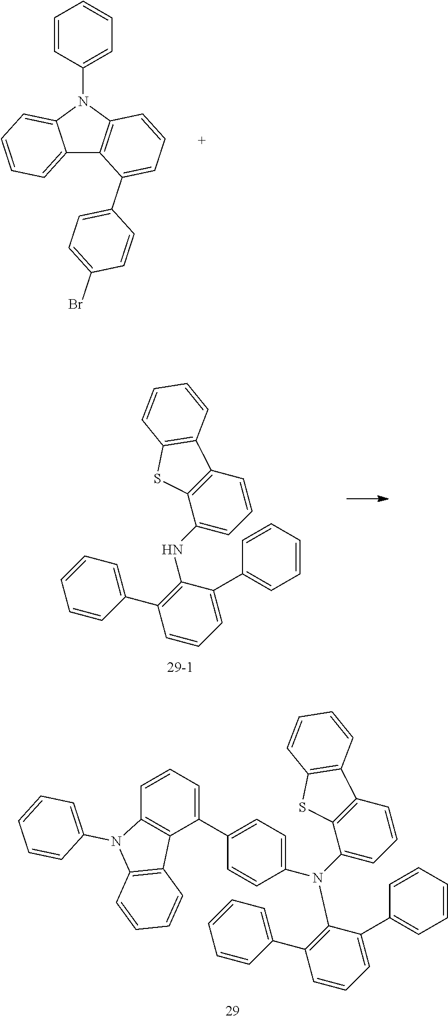 [SMILES: Brc1ccc(-c2cccc3c2c2ccccc2n3-c2ccccc2)cc1 ; c1ccc(-c2cccc(-c3ccccc3)c2Nc2cccc3c2sc2ccccc23)cc1 ; c1ccc(-c2cccc(-c3ccccc3)c2N(c2ccc(-c3cccc4c3c3ccccc3n4-c3ccccc3)cc2)c2cccc3c2sc2ccccc23)cc1]

Toluene 150 ml was added to 4-4-Bromophenyl-N-phenylcarbazole (10.0 g, 0.025 mol), Intermediate 29-1 (16.1 g, 0.038 mol), NaOtBu (4.8 g, 0.050 mol), Pd(dba)₂ (0.7 g, 0.003 mol), t-Bu₃P (0.5 g, 0.006 mol), and then was reacted with stirring at 70° C. for 4 hours. After completion of the reaction, the reaction mixture was extracted, concentrated, and then subjected to column and recrystallization to obtain 13.1 g (yield 70.0%) of Compound 29.

LC/MS: m/z=744[(M)⁺]

### Synthesis Example 7: Synthesis of Compound 42

### (1) Preparative Example 1: Synthesis of Compound 42

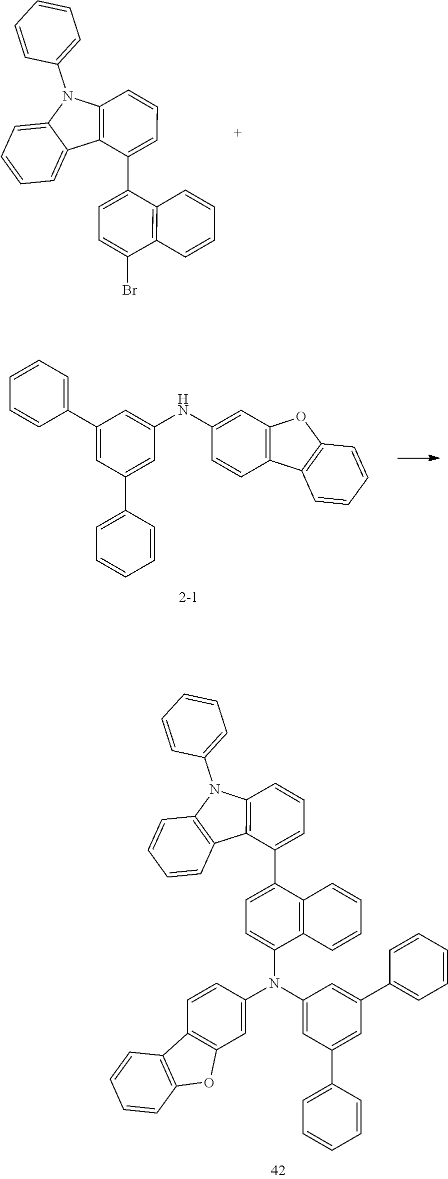 [SMILES: Brc1ccc(-c2cccc3c2c2ccccc2n3-c2ccccc2)c2ccccc12 ; c1ccc(-c2cc(Nc3ccc4c(c3)oc3ccccc34)cc(-c3ccccc3)c2)cc1 ; c1ccc(-c2cc(-c3ccccc3)cc(N(c3ccc4c(c3)oc3ccccc34)c3ccc(-c4cccc5c4c4ccccc4n5-c4ccccc4)c4ccccc34)c2)cc1]

Toluene 150 ml was added to 4-(4-bromonaphthalen-1-yl)-9-phenyl-9H-carbazole (10.0 g, 0.022 mol), Intermediate 2-1 (13.8 g, 0.034 mol), NaOtBu (4.3 g, 0.045 mol), Pd(dba)₂ (0.6 g, 0.002 mol), t-Bu₃P (0.5 g, 0.006 mol), and then was reacted with stirring at 70° C. for 4 hours. After completion of the reaction, the reaction mixture was extracted, concentrated, and then subjected to column and recrystallization to obtain 12.9 g (yield 74.3%) of Compound 42.

LC/MS: m/z=778[(M)⁺]

### Synthesis Example 8: Synthesis of Compound 63

### (1) Preparative Example 1: Synthesis of Intermediate 63-1

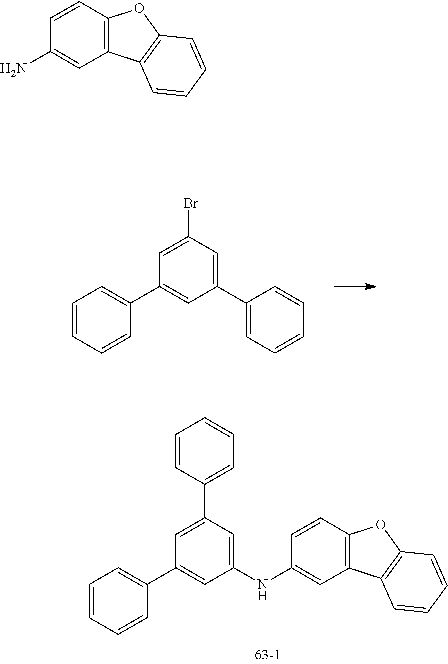 [SMILES: Nc1ccc2oc3ccccc3c2c1 ; Brc1cc(-c2ccccc2)cc(-c2ccccc2)c1 ; c1ccc(-c2cc(Nc3ccc4oc5ccccc5c4c3)cc(-c3ccccc3)c2)cc1]

Toluene 150 ml was added to Dibenzo[b,d]furan-2-amine (10.0 g, 0.055 mol), 1-Bromo-3,5-diphenylbenzene (25.3 g, 0.082 mol), NaOtBu (10.5 g, 0.109 mol), Pd(dba)₂ (1.6 g, 0.003 mol), t-Bu₃P (1.1 g, 0.006 mol), and then were reacted with stirring at 70° C. for 4 hours. After completion of the reaction, the reaction mixture was extracted, concentrated, and then subjected to column and recrystallization to obtain 12.4 g (yield 55.2%) of Compound 63-1.

### (2) Preparative Example 2: Synthesis of Compound 63

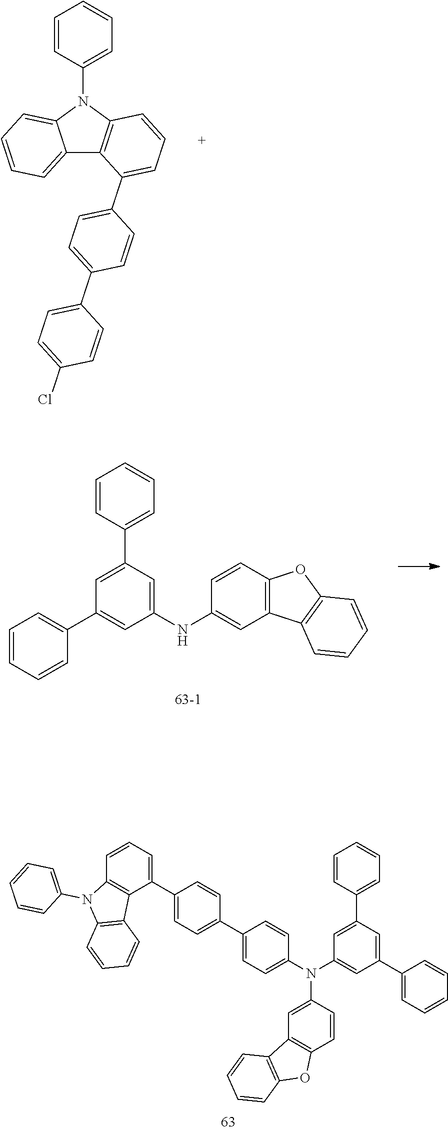 [SMILES: Clc1ccc(-c2ccc(-c3cccc4c3c3ccccc3n4-c3ccccc3)cc2)cc1 ; c1ccc(-c2cc(Nc3ccc4oc5ccccc5c4c3)cc(-c3ccccc3)c2)cc1 ; c1ccc(-c2cc(-c3ccccc3)cc(N(c3ccc(-c4ccc(-c5cccc6c5c5ccccc5n6-c5ccccc5)cc4)cc3)c3ccc4oc5ccccc5c4c3)c2)cc1]

Toluene 150 ml was added to 4-(4′-chlorobiphenyl-4-yl)-9-phenyl-9H-carbazole (10.0 g, 0.023 mol), Intermediate 63-1 (14.4 g, 0.035 mol), NaOtBu (4.5 g, 0.047 mol), Pd(dba)₂ (0.7 g, 0.002 mol), t-Bu₃P (0.5 g, 0.006 mol), and then was reacted with stirring at 70ºC for 4 hours. After completion of the reaction, the reaction mixture was extracted, concentrated, and then subjected to column and recrystallization to obtain 14.1 g (yield 75.3%) of Compound 63.

LC/MS: m/z=804[(M)⁺]

### Synthesis Example 9: Synthesis of Compound 77

### (1) Preparative Example 1: Synthesis of Intermediate 77-1

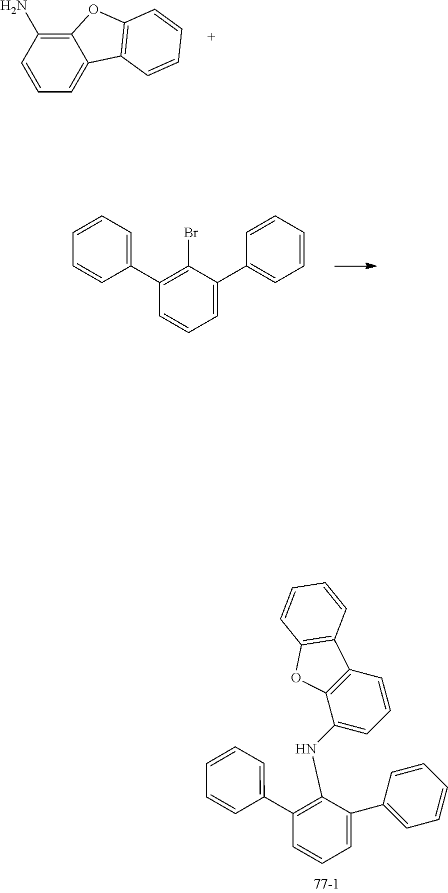 [SMILES: Nc1cccc2c1oc1ccccc12 ; Brc1c(-c2ccccc2)cccc1-c1ccccc1 ; c1ccc(-c2cccc(-c3ccccc3)c2Nc2cccc3c2oc2ccccc23)cc1]

Toluene 150 ml was added to Dibenzo[b,d]furan-4-amine (10.0 g, 0.055 mol), 1-Bromo-3,5-diphenylbenzene (25.3 g, 0.082 mol), NaOtBu (10.5 g, 0.109 mol), Pd(dba)₂ (1.6 g, 0.003 mol), t-Bu₃P (1.1 g, 0.006 mol), and then was reacted with stirring at 70° C. for 4 hours. After completion of the reaction, the reaction mixture was extracted, concentrated, and then subjected to column and recrystallization to obtain 10.8 g (yield 48.1%) of Intermediate 77-1.

### (2) Preparative Example 2: Synthesis of Compound 77

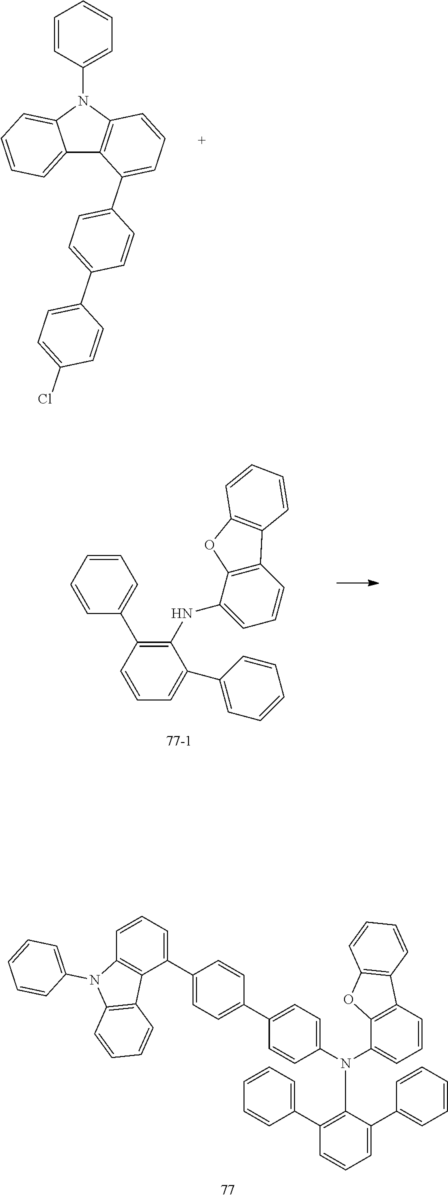 [SMILES: Clc1ccc(-c2ccc(-c3cccc4c3c3ccccc3n4-c3ccccc3)cc2)cc1 ; c1ccc(-c2cccc(-c3ccccc3)c2Nc2cccc3c2oc2ccccc23)cc1 ; c1ccc(-c2cccc(-c3ccccc3)c2N(c2ccc(-c3ccc(-c4cccc5c4c4ccccc4n5-c4ccccc4)cc3)cc2)c2cccc3c2oc2ccccc23)cc1]

Toluene 150 ml was added to 4-(4′-chlorobiphenyl-4-yl)-9-phenyl-9H-carbazole (10.0 g, 0.023 mol), Intermediate 77-1 (14.4 g, 0.035 mol), NaOtBu (4.5 g, 0.047 mol), Pd(dba)₂ (0.7 g, 0.002 mol), t-Bu₃P (0.5 g, 0.006 mol), and then was reacted with stirring at 70ºC for 4 hours. After completion of the reaction, the reaction mixture was extracted, concentrated, and then subjected to column and recrystallization to obtain 10.2 g (yield 54.5%) of Compound 77.

LC/MS: m/z=804[(M)⁺]

### Synthesis Example 10: Synthesis of Compound 91

### (1) Preparative Example 1: Synthesis of Intermediate 91-1

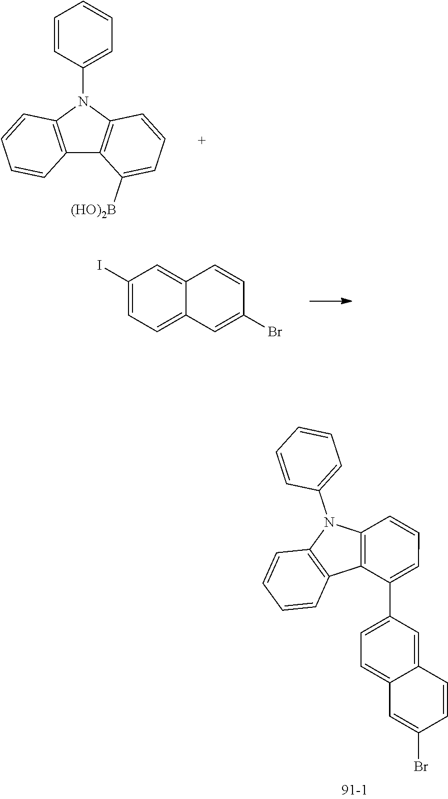 [SMILES: OB(O)c1cccc2c1c1ccccc1n2-c1ccccc1 ; Brc1ccc2cc(I)ccc2c1 ; Brc1ccc2cc(-c3cccc4c3c3ccccc3n4-c3ccccc3)ccc2c1]

Toluene 200 mL, ethanol 50 mL, and H₂O 50 mL were added to (9-Phenyl-9H-carbazol-4-yl)boronic acid (10.0 g, 0.035 mol), 2-Bromo-6-iodonaphthalene (13.9 g, 0.042 mol), K₂CO₃ (14.4 g, 0.105 mol), Pd(PPh₃)₄ (0.8 g, 0.0007 mol), and then was reacted with stirring at 100° C. for 6 hours. After completion of the reaction, the reaction mixture was extracted, concentrated, and then subjected to column and recrystallization to obtain 9.5 g (yield 60.8%) of Intermediate 91-1.

### (2) Preparative Example 2: Synthesis of Intermediate 91-2

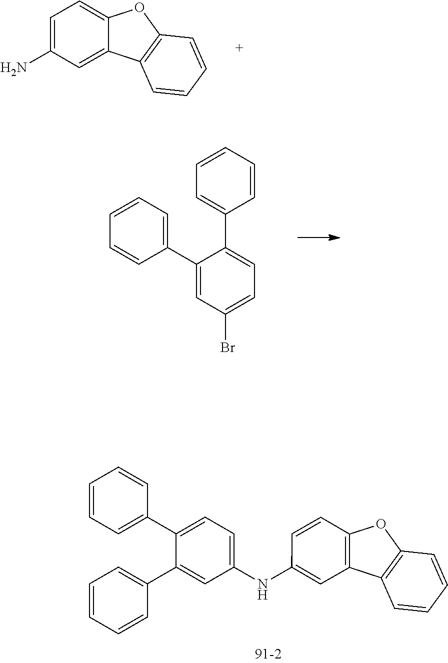 [SMILES: Nc1ccc2oc3ccccc3c2c1 ; Brc1ccc(-c2ccccc2)c(-c2ccccc2)c1 ; c1ccc(-c2ccc(Nc3ccc4oc5ccccc5c4c3)cc2-c2ccccc2)cc1]

Toluene 150 ml was added to Dibenzo[b,d]furan-2-amine (10.0 g, 0.055 mol), 1-Bromo-3.4-diphenylbenzene (25.3 g, 0.082 mol), NaOtBu (10.5 g, 0.109 mol), Pd(dba)₂ (1.6 g, 0.003 mol), t-Bu₃P (1.1 g, 0.006 mol), and then was reacted with stirring at 70° C. for 4 hours. After completion of the reaction, the reaction mixture was extracted, concentrated, and then subjected to column and recrystallization to obtain 9.7 g (yield 43.2%) of Intermediate 91-2.

### (3) Preparative Example 3: Synthesis of Compound 91

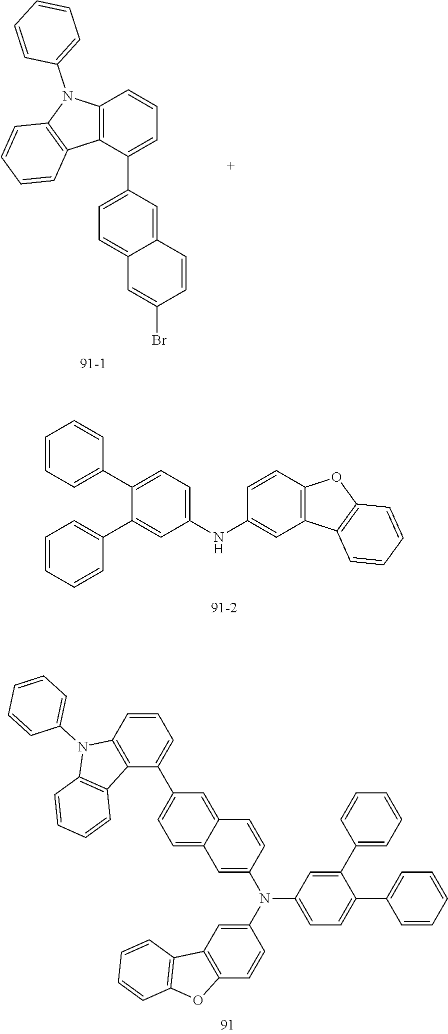 [SMILES: Brc1ccc2cc(-c3cccc4c3c3ccccc3n4-c3ccccc3)ccc2c1 ; c1ccc(-c2ccc(Nc3ccc4oc5ccccc5c4c3)cc2-c2ccccc2)cc1 ; c1ccc(-c2ccc(N(c3ccc4cc(-c5cccc6c5c5ccccc5n6-c5ccccc5)ccc4c3)c3ccc4oc5ccccc5c4c3)cc2-c2ccccc2)cc1]

Toluene 150 ml was added to Intermediate 91-1 (10.0 g, 0.022 mol), Intermediate 91-2 (13.8 g, 0.034 mol), NaOtBu (4.3 g, 0.045 mol), Pd(dba)₂ (0.6 g, 0.002 mol), t-Bu₃P (0.5 g, 0.006 mol), and then was reacted with stirring at 70° C. for 4 hours. After completion of the reaction, the reaction mixture was extracted, concentrated, and then subjected to column and recrystallization to obtain 10.5 g (yield 60.4%) of Compound 91.

LC/MS: m/z=778[(M)⁺]

### Synthesis Example 11: Synthesis of Compound 106

### (1) Preparative Example 1: Synthesis of Intermediate 106-1

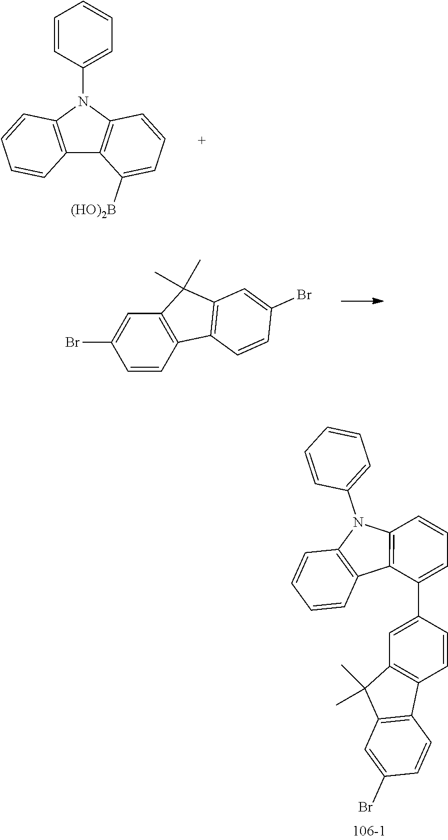 [SMILES: OB(O)c1cccc2c1c1ccccc1n2-c1ccccc1 ; CC1(C)c2cc(Br)ccc2-c2ccc(Br)cc21 ; CC1(C)c2cc(Br)ccc2-c2ccc(-c3cccc4c3c3ccccc3n4-c3ccccc3)cc21]

Toluene 200 mL, ethanol 50 mL, and H₂O 50 mL were added to (9-Phenyl-9H-carbazol-4-yl)boronic acid (10.0 g, 0.035 mol), 2.7-Dibromo-9.9-dimethylfluorene (14.7 g, 0.042 mol), K₂CO₃ (14.4 g, 0.105 mol), Pd(PPh₃)₄ (0.8 g, 0.0007 mol), and then were reacted with stirring at 100° C. for 6 hours. After completion of the reaction, the reaction mixture was extracted, concentrated, and then subjected to column and recrystallization to obtain 9.5 g (yield 53.0%) of Intermediate 106-1.

### (2) Preparative Example 2: Synthesis of Compound 106

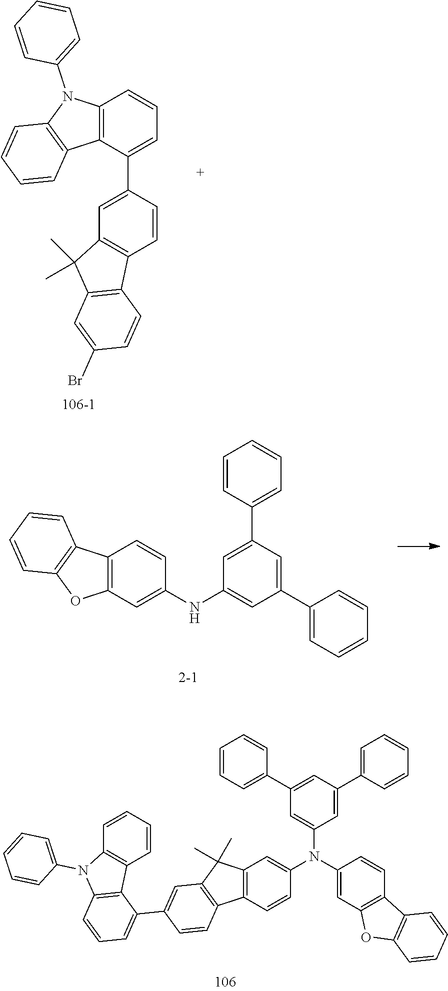 [SMILES: CC1(C)c2cc(Br)ccc2-c2ccc(-c3cccc4c3c3ccccc3n4-c3ccccc3)cc21 ; c1ccc(-c2cc(Nc3ccc4c(c3)oc3ccccc34)cc(-c3ccccc3)c2)cc1 ; CC1(C)c2cc(-c3cccc4c3c3ccccc3n4-c3ccccc3)ccc2-c2ccc(N(c3cc(-c4ccccc4)cc(-c4ccccc4)c3)c3ccc4c(c3)oc3ccccc34)cc21]

Toluene 150 m was added to Intermediate 106-1 (10.0 g, 0.019 mol), Intermediate 2-1 (12.0 g, 0.029 mol), NaOtBu (3.7 g, 0.039 mol), Pd(dba)₂ (0.6 g, 0.002 mol), t-Bu₃P (0.4 g, 0.006 mol), and then was reacted with stirring at 70° C. for 4 hours. After completion of the reaction, the reaction mixture was extracted, concentrated, and then subjected to column and recrystallization to obtain 11.9 g (yield 72.5%) of Compound 106.

LC/MS: m/z=844[(M)⁺]

### DEVICE EXAMPLES

In an example of the present invention, an ITO transparent electrode was patterned on a glass substrate having dimensions of 25 mm×25 mm×0.7 mm to have a light emitting area of 2 mm×2 mm using an ITO glass substrate to which the ITO transparent electrode was attached, followed by cleaning. After the substrate was mounted in a vacuum chamber, organic materials and metals were deposited in the following structure on the ITO at a base pressure of 1×10⁻⁶ torr.

### Device Examples 1 to 73

After fabricating an organic light emitting device with the following device structure using the compounds implemented according to an embodiment of the present invention as a material for an electron blocking layer, luminescent properties including a current efficiency were measured.

ITO/Hole injecting layer (HAT-CN, 5 nm)/Hole transport layer (α-NPB, 100 nm)/Electron blocking layer (EBL1 10 nm)/Light emitting layer (20 nm)/Electron transport layer (201:Liq, 30 nm)/LiF (1 nm)/Al (100 nm)

In order to form a hole injecting layer on the ITO transport electrode, HAT-CN was deposited with a thickness of 5 nm and then a 100 nm thick hole transport layer was formed into a film using α-NPB. A 10 nm thick electron blocking layer was formed into a film using a compound according to an embodiment of the present invention listed in Table 1 below. Further, a 20 nm thick light emitting layer was co-deposited using BH1 as a host compound and BD1 as a dopant compound. Further, a 30 nm thick electron transport layer (doped with Liq 50% of the following Compound 201) was formed into a film. Finally, a 1 nm thick LiF and 100 nm thick Al were formed into films to fabricate an organic light emitting device.

### Device Comparative Example 1

An organic light emitting device for Device Comparative Example 1 was fabricated in the same manner, except that following EB1 was used instead of the compound according to an embodiment of the present invention as an electron blocking layer in the device structure of the above Example 1.

### Device Comparative Example 2

An organic light emitting device for Device Comparative Example 2 was fabricated in the same manner, except that following EB2 was used instead of the compound according to an embodiment of the present invention as an electron blocking layer in the device structure of the above Example 1.

### Device Comparative Example 3

An organic light emitting device for Device Comparative Example 3 was fabricated in the same manner, except that following EB3 was used instead of the compound according to an embodiment of the present invention as an electron blocking layer in the device structure of the above Example 1.

### Device Comparative Example 4

An organic light emitting device for Device Comparative Example 4 was fabricated in the same manner, except that following EB4 was used instead of the compound according to an embodiment of the present invention as an electron blocking layer in the device structure of the above Example 1.

### Experimental Example 1: Luminescent Properties of Device Examples 1 to 73

Voltages, currents, and luminous efficiencies of the organic light emitting devices fabricated according to the above Examples and Comparative Examples were measured using Source meter (Model 237, Keithley) and a spectroradiometer (PR-650, Photo Research). Result values at 1000 nit are shown in Table 1 below.

TABLE 1

| | Electron | | | | |
|---|---|---|---|---|---|
| Examples | blocking layer | V | cd/A | CIEx | CIEy |
| 1 | Formula 1 | 4.6 | 8.4 | 0.1330 | 0.1235 |
| 2 | Formula 2 | 4.5 | 8.2 | 0.1380 | 0.1363 |
| 3 | Formula 3 | 4.6 | 8.1 | 0.1370 | 0.1460 |
| 4 | Formula 4 | 4.3 | 8.5 | 0.1380 | 0.1410 |
| 5 | Formula 5 | 4.2 | 8.3 | 0.1310 | 0.1330 |
| 6 | Formula 6 | 4.5 | 8.1 | 0.1370 | 0.1420 |
| 7 | Formula 7 | 4.6 | 8.5 | 0.1380 | 0.1460 |
| 8 | Formula 8 | 4.4 | 8.1 | 0.1310 | 0.1420 |
| 9 | Formula 9 | 4.6 | 8.2 | 0.1350 | 0.1335 |
| 10 | Formula 12 | 4.4 | 8.5 | 0.1320 | 0.1292 |
| 11 | Formula 17 | 4.1 | 8.1 | 0.1310 | 0.1328 |
| 12 | Formula 18 | 4.3 | 8.4 | 0.1320 | 0.1235 |
| 13 | Formula 20 | 4.8 | 8.3 | 0.1360 | 0.1351 |
| 14 | Formula 21 | 4.5 | 8.1 | 0.1316 | 0.1340 |
| 15 | Formula 22 | 4.7 | 8.5 | 0.1345 | 0.1322 |
| 16 | Formula 24 | 4.7 | 8.6 | 0.1331 | 0.1234 |
| 17 | Formula 25 | 4.6 | 8.5 | 0.1393 | 0.1244 |
| 18 | Formula 27 | 4.5 | 8.2 | 0.1334 | 0.1328 |
| 19 | Formula 28 | 4.7 | 8.4 | 0.1353 | 0.1335 |
| 20 | Formula 29 | 4.8 | 8.2 | 0.1324 | 0.1301 |
| 21 | Formula 33 | 4.7 | 8.3 | 0.1345 | 0.1294 |
| 22 | Formula 34 | 4.8 | 8.5 | 0.1339 | 0.1232 |
| 23 | Formula 35 | 4.3 | 8.4 | 0.1350 | 0.1192 |
| 24 | Formula 36 | 4.5 | 8.4 | 0.1321 | 0.1221 |
| 25 | Formula 37 | 4.3 | 8.2 | 0.1326 | 0.1254 |
| 26 | Formula 40 | 4.6 | 8.1 | 0.1313 | 0.1293 |
| 27 | Formula 41 | 4.3 | 8.4 | 0.1320 | 0.1262 |
| 28 | Formula 42 | 4.5 | 8.2 | 0.1345 | 0.1333 |
| 29 | Formula 43 | 4.3 | 8.1 | 0.1350 | 0.1266 |
| 30 | Formula 44 | 4.2 | 8.4 | 0.1320 | 0.1353 |
| 31 | Formula 45 | 4.8 | 8.5 | 0.1310 | 0.1470 |
| 32 | Formula 48 | 4.6 | 8.6 | 0.1300 | 0.1470 |
| 33 | Formula 49 | 4.8 | 8.4 | 0.1390 | 0.1450 |
| 34 | Formula 50 | 4.7 | 8.2 | 0.1330 | 0.1420 |
| 35 | Formula 52 | 4.5 | 8.4 | 0.1310 | 0.1460 |
| 36 | Formula 53 | 4.6 | 8.5 | 0.1310 | 0.1420 |
| 37 | Formula 54 | 4.5 | 8.7 | 0.1340 | 0.1430 |
| 38 | Formula 55 | 4.7 | 8.1 | 0.1353 | 0.1420 |
| 39 | Formula 57 | 4.6 | 8.4 | 0.1356 | 0.1430 |
| 40 | Formula 58 | 4.2 | 8.5 | 0.1321 | 0.1440 |
| 41 | Formula 60 | 4.7 | 8.2 | 0.1347 | 0.1480 |
| 42 | Formula 61 | 4.8 | 8.7 | 0.1340 | 0.1480 |
| 43 | Formula 62 | 4.4 | 8.8 | 0.1312 | 0.1470 |
| 44 | Formula 63 | 4.6 | 8.0 | 0.1327 | 0.1530 |
| 45 | Formula 66 | 4.5 | 8.6 | 0.1353 | 0.1520 |
| 46 | Formula 68 | 4.8 | 8.1 | 0.1359 | 0.1450 |
| 47 | Formula 69 | 4.4 | 8.5 | 0.1353 | 0.1490 |
| 48 | Formula 70 | 4.5 | 8.1 | 0.1310 | 0.1420 |
| 49 | Formula 73 | 4.6 | 8.4 | 0.1376 | 0.1450 |
| 50 | Formula 74 | 4.5 | 8.5 | 0.1320 | 0.1480 |
| 51 | Formula 75 | 4.1 | 8.2 | 0.1340 | 0.1470 |
| 52 | Formula 76 | 4.3 | 8.5 | 0.1320 | 0.1480 |
| 53 | Formula 77 | 4.5 | 8.1 | 0.1320 | 0.1450 |
| 54 | Formula 78 | 4.2 | 8.4 | 0.1330 | 0.1460 |
| 55 | Formula 79 | 4.1 | 8.2 | 0.1290 | 0.1470 |
| 56 | Formula 83 | 4.3 | 8.5 | 0.1310 | 0.1530 |
| 57 | Formula 85 | 4.3 | 8.2 | 0.1330 | 0.1480 |
| 58 | Formula 88 | 4.5 | 8.5 | 0.1310 | 0.1450 |
| 59 | Formula 90 | 4.1 | 8.2 | 0.1320 | 0.1520 |
| 60 | Formula 91 | 4.5 | 8.3 | 0.1330 | 0.1460 |
| 61 | Formula 92 | 4.7 | 8.3 | 0.1320 | 0.1460 |
| 62 | Formula 93 | 4.3 | 8.5 | 0.1310 | 0.1490 |
| 63 | Formula 94 | 4.1 | 8.1 | 0.1320 | 0.1470 |
| 64 | Formula 95 | 4.6 | 8.1 | 0.1340 | 0.1440 |
| 65 | Formula 98 | 4.2 | 8.5 | 0.1330 | 0.1440 |
| 66 | Formula 99 | 4.1 | 8.7 | 0.1320 | 0.1480 |
| 67 | Formula 100 | 4.6 | 8.6 | 0.1310 | 0.1470 |
| 68 | Formula 105 | 4.7 | 8.5 | 0.1320 | 0.1420 |
| 69 | Formula 106 | 4.3 | 8.5 | 0.1330 | 0.1470 |
| 70 | Formula 107 | 4.0 | 8.4 | 0.1330 | 0.1430 |
| 71 | Formula 108 | 4.4 | 8.2 | 0.1330 | 0.1450 |
| 72 | Formula 109 | 4.1 | 8.3 | 0.1330 | 0.1460 |
| 73 | Formula 110 | 4.5 | 8.5 | 0.1310 | 0.1510 |
| Comparative | EB1 | 5.0 | 7.8 | 0.1328 | 0.1328 |
| Example 1 | | | | | |
| Comparative | EB2 | 4.9 | 7.9 | 0.1342 | 0.1346 |
| Example 2 | | | | | |
| Comparative | EB3 | 4.9 | 7.7 | 0.1333 | 0.1340 |
| Example 3 | | | | | |
| Comparative | EB4 | 5.1 | 7.8 | 0.1330 | 0.1338 |
| Example 4 | | | | | |

As seen from the results in Table 1, it was confirmed that when the compound according to the embodiment of the present invention was employed for the electron blocking layer in the organic light emitting device, the luminescent properties such as low-voltage driving characteristic, the luminous efficiency, and the quantum efficiency are much more excellent than the devices (Comparative Examples 1 to 4) which employ the compound compared to the characteristic structure of the compound according to the embodiment of the present invention as a compound used for a material of the electron blocking layer of the related art.

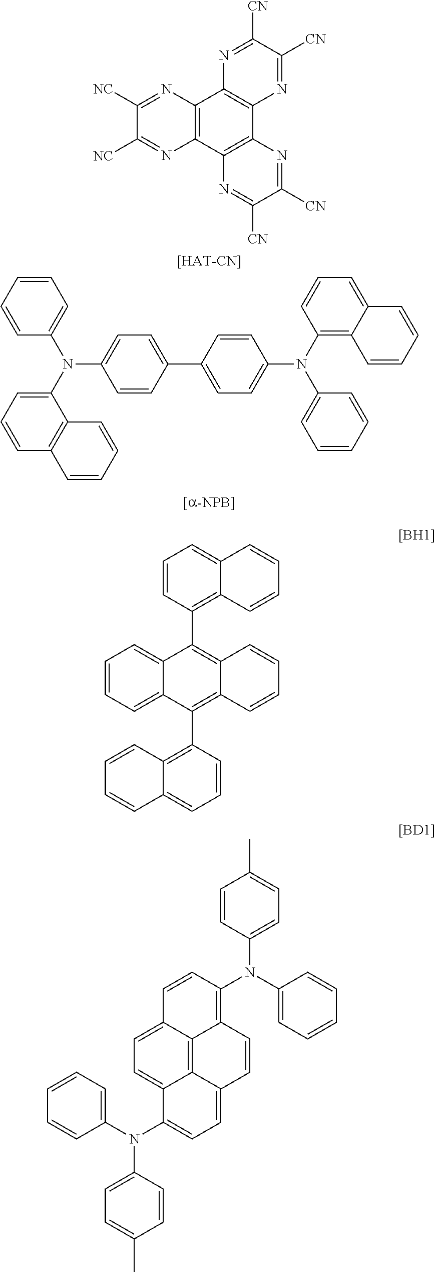

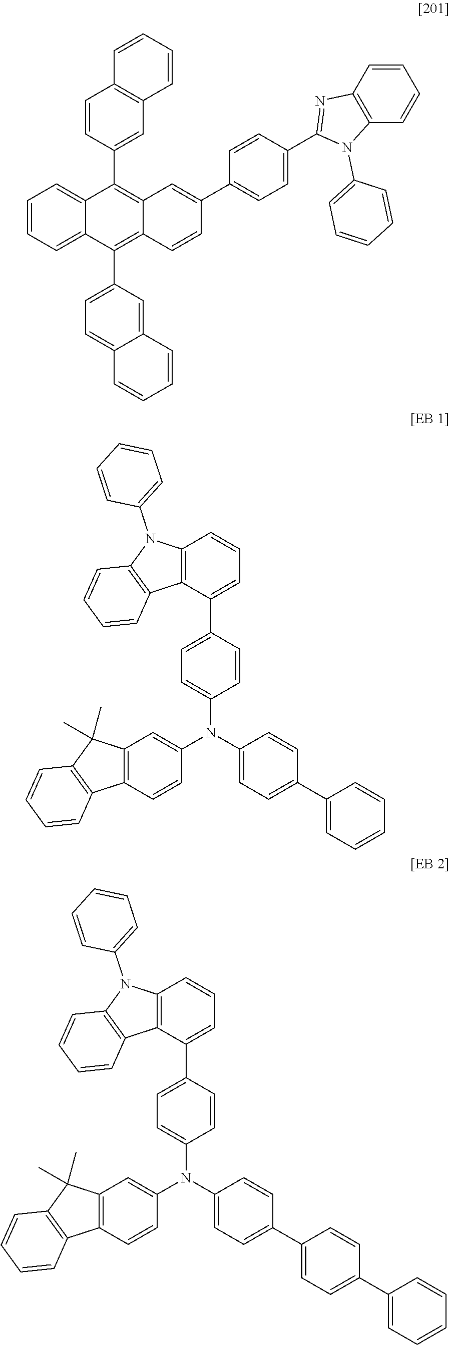 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC1(C)c2ccccc2-c2ccc(N(c3ccc(-c4ccccc4)cc3)c3ccc(-c4cccc5c4c4ccccc4n5-c4ccccc4)cc3)cc21 ; CC1(C)c2ccccc2-c2ccc(N(c3ccc(-c4ccc(-c5ccccc5)cc4)cc3)c3ccc(-c4cccc5c4c4ccccc4n5-c4ccccc4)cc3)cc21]

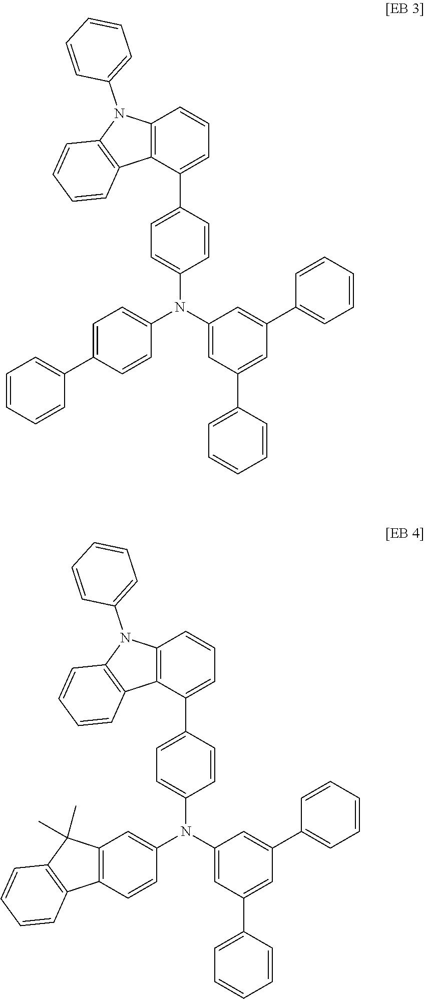 [SMILES: c1ccc(-c2ccc(N(c3ccc(-c4cccc5c4c4ccccc4n5-c4ccccc4)cc3)c3cc(-c4ccccc4)cc(-c4ccccc4)c3)cc2)cc1 ; CC1(C)c2ccccc2-c2ccc(N(c3ccc(-c4cccc5c4c4ccccc4n5-c4ccccc4)cc3)c3cc(-c4ccccc4)cc(-c4ccccc4)c3)cc21]

### INDUSTRIAL APPLICABILITY

An organic light emitting compound according to the embodiment of the present invention is employed as a material for an organic layer such as an electron blocking layer, in the organic light emitting device to implement an organic light emitting device having light emission characteristics such as low-voltage driving and excellent luminous efficiency, thus can be advantageously used industrially in a variety of display devices.

1. An organic light emitting compound represented by Formula I below: 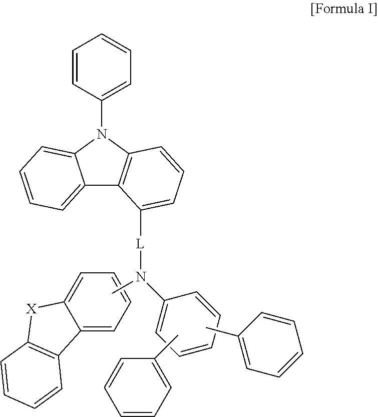
wherein in Formula I, X is O or S and L is a divalent linking group of any one selected from groups represented by Structural Formula 1 below.

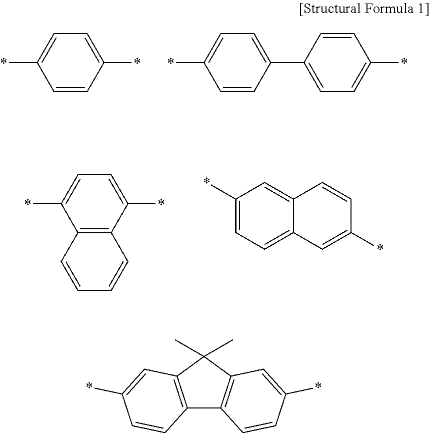

2. The organic light emitting device of claim 1, wherein Formula I is any one selected from Compound 1 to Compound 112 below.

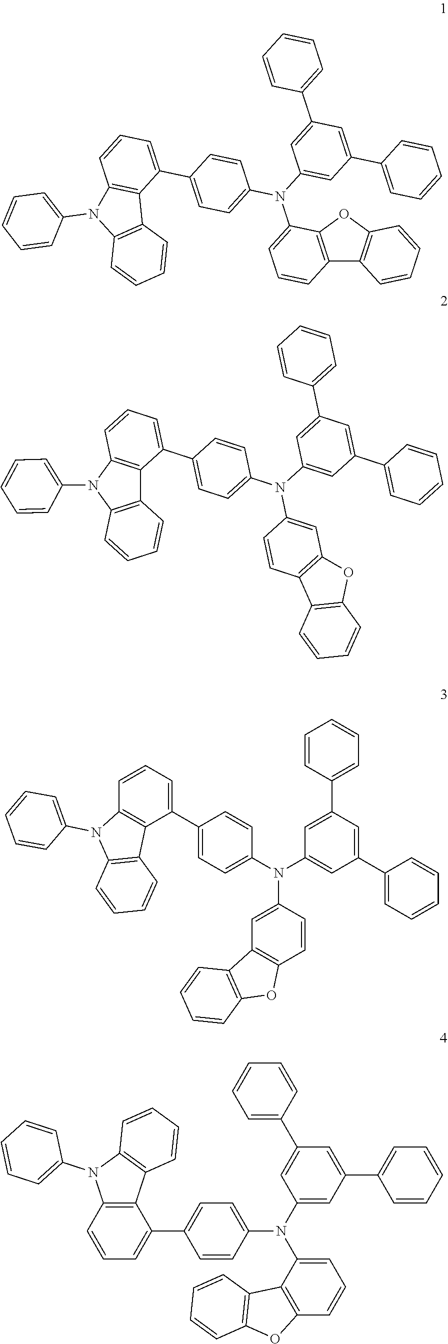

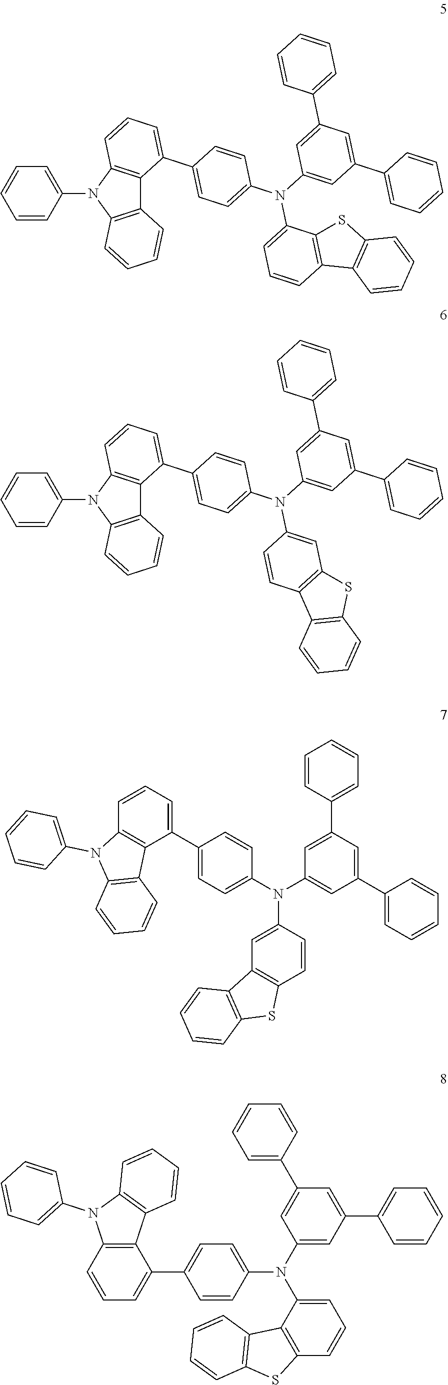 [SMILES: c1ccc(-c2cc(-c3ccccc3)cc(N(c3ccc(-c4cccc5c4c4ccccc4n5-c4ccccc4)cc3)c3cccc4c3sc3ccccc34)c2)cc1 ; c1ccc(-c2cc(-c3ccccc3)cc(N(c3ccc(-c4cccc5c4c4ccccc4n5-c4ccccc4)cc3)c3ccc4c(c3)sc3ccccc34)c2)cc1 ; c1ccc(-c2cc(-c3ccccc3)cc(N(c3ccc(-c4cccc5c4c4ccccc4n5-c4ccccc4)cc3)c3ccc4sc5ccccc5c4c3)c2)cc1 ; c1ccc(-c2cc(-c3ccccc3)cc(N(c3ccc(-c4cccc5c4c4ccccc4n5-c4ccccc4)cc3)c3cccc4sc5ccccc5c34)c2)cc1]

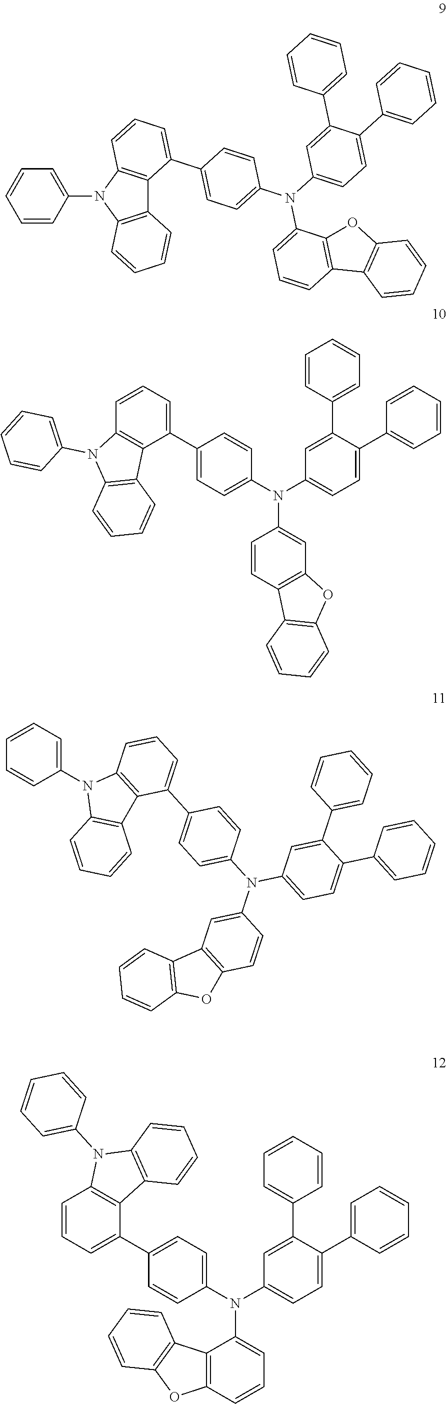 [SMILES: c1ccc(-c2ccc(N(c3ccc(-c4cccc5c4c4ccccc4n5-c4ccccc4)cc3)c3cccc4c3oc3ccccc34)cc2-c2ccccc2)cc1 ; c1ccc(-c2ccc(N(c3ccc(-c4cccc5c4c4ccccc4n5-c4ccccc4)cc3)c3ccc4c(c3)oc3ccccc34)cc2-c2ccccc2)cc1 ; c1ccc(-c2ccc(N(c3ccc(-c4cccc5c4c4ccccc4n5-c4ccccc4)cc3)c3ccc4oc5ccccc5c4c3)cc2-c2ccccc2)cc1 ; c1ccc(-c2ccc(N(c3ccc(-c4cccc5c4c4ccccc4n5-c4ccccc4)cc3)c3cccc4oc5ccccc5c34)cc2-c2ccccc2)cc1]

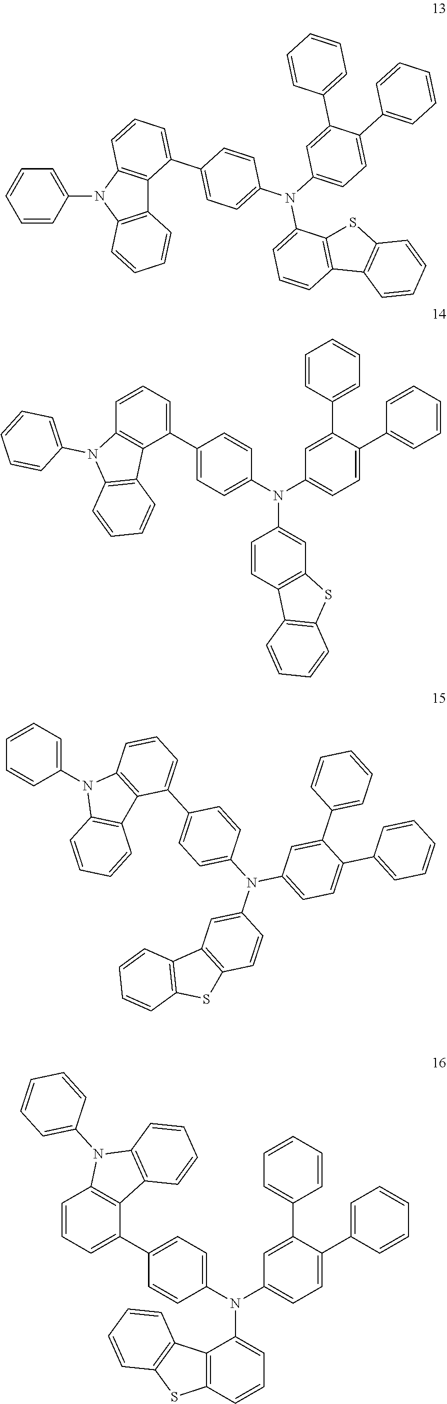 [SMILES: c1ccc(-c2ccc(N(c3ccc(-c4cccc5c4c4ccccc4n5-c4ccccc4)cc3)c3cccc4c3sc3ccccc34)cc2-c2ccccc2)cc1 ; c1ccc(-c2ccc(N(c3ccc(-c4cccc5c4c4ccccc4n5-c4ccccc4)cc3)c3ccc4c(c3)sc3ccccc34)cc2-c2ccccc2)cc1 ; c1ccc(-c2ccc(N(c3ccc(-c4cccc5c4c4ccccc4n5-c4ccccc4)cc3)c3ccc4sc5ccccc5c4c3)cc2-c2ccccc2)cc1 ; c1ccc(-c2ccc(N(c3ccc(-c4cccc5c4c4ccccc4n5-c4ccccc4)cc3)c3cccc4sc5ccccc5c34)cc2-c2ccccc2)cc1]

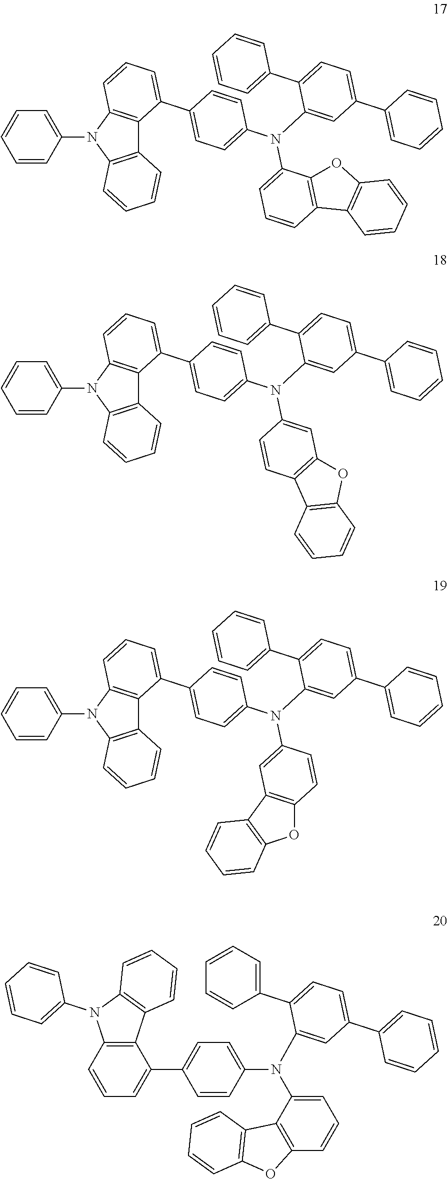 [SMILES: c1ccc(-c2ccc(-c3ccccc3)c(N(c3ccc(-c4cccc5c4c4ccccc4n5-c4ccccc4)cc3)c3cccc4c3oc3ccccc34)c2)cc1 ; c1ccc(-c2ccc(-c3ccccc3)c(N(c3ccc(-c4cccc5c4c4ccccc4n5-c4ccccc4)cc3)c3ccc4c(c3)oc3ccccc34)c2)cc1 ; c1ccc(-c2ccc(-c3ccccc3)c(N(c3ccc(-c4cccc5c4c4ccccc4n5-c4ccccc4)cc3)c3ccc4oc5ccccc5c4c3)c2)cc1 ; c1ccc(-c2ccc(-c3ccccc3)c(N(c3ccc(-c4cccc5c4c4ccccc4n5-c4ccccc4)cc3)c3cccc4oc5ccccc5c34)c2)cc1]

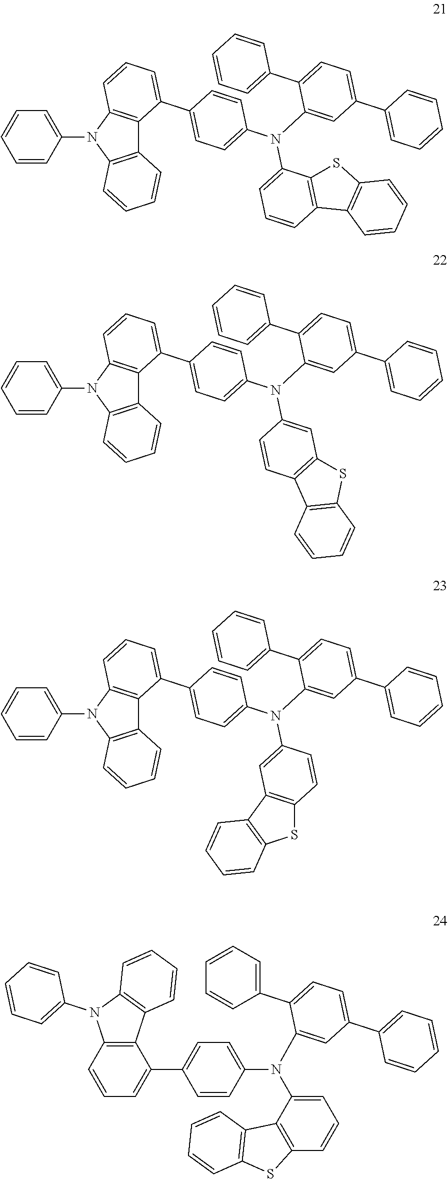 [SMILES: c1ccc(-c2ccc(-c3ccccc3)c(N(c3ccc(-c4cccc5c4c4ccccc4n5-c4ccccc4)cc3)c3cccc4c3sc3ccccc34)c2)cc1 ; c1ccc(-c2ccc(-c3ccccc3)c(N(c3ccc(-c4cccc5c4c4ccccc4n5-c4ccccc4)cc3)c3ccc4c(c3)sc3ccccc34)c2)cc1 ; c1ccc(-c2ccc(-c3ccccc3)c(N(c3ccc(-c4cccc5c4c4ccccc4n5-c4ccccc4)cc3)c3ccc4sc5ccccc5c4c3)c2)cc1 ; c1ccc(-c2ccc(-c3ccccc3)c(N(c3ccc(-c4cccc5c4c4ccccc4n5-c4ccccc4)cc3)c3cccc4sc5ccccc5c34)c2)cc1]

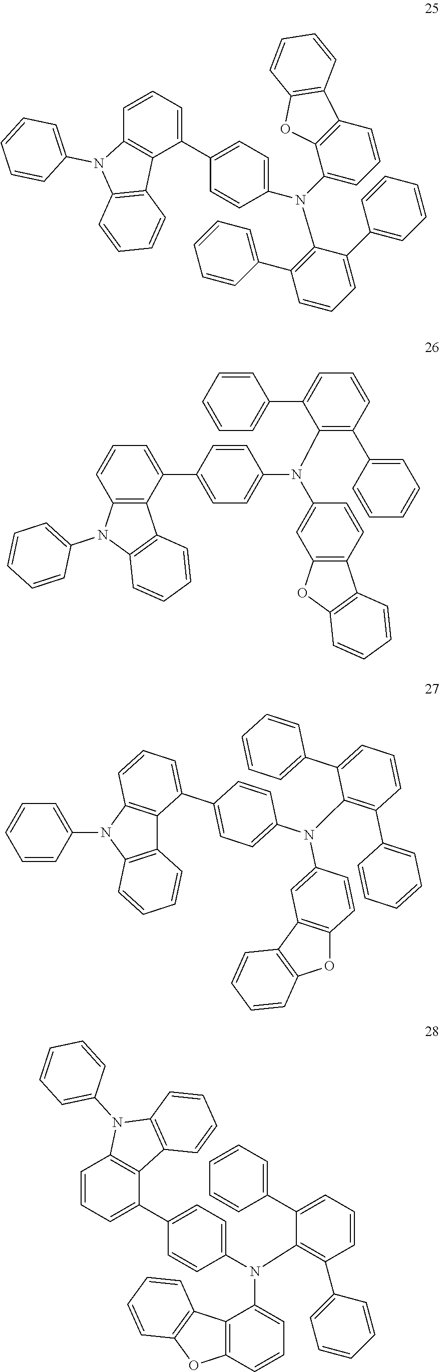 [SMILES: c1ccc(-c2cccc(-c3ccccc3)c2N(c2ccc(-c3cccc4c3c3ccccc3n4-c3ccccc3)cc2)c2cccc3c2oc2ccccc23)cc1 ; c1ccc(-c2cccc(-c3ccccc3)c2N(c2ccc(-c3cccc4c3c3ccccc3n4-c3ccccc3)cc2)c2ccc3c(c2)oc2ccccc23)cc1 ; c1ccc(-c2cccc(-c3ccccc3)c2N(c2ccc(-c3cccc4c3c3ccccc3n4-c3ccccc3)cc2)c2ccc3oc4ccccc4c3c2)cc1 ; c1ccc(-c2cccc(-c3ccccc3)c2N(c2ccc(-c3cccc4c3c3ccccc3n4-c3ccccc3)cc2)c2cccc3oc4ccccc4c23)cc1]

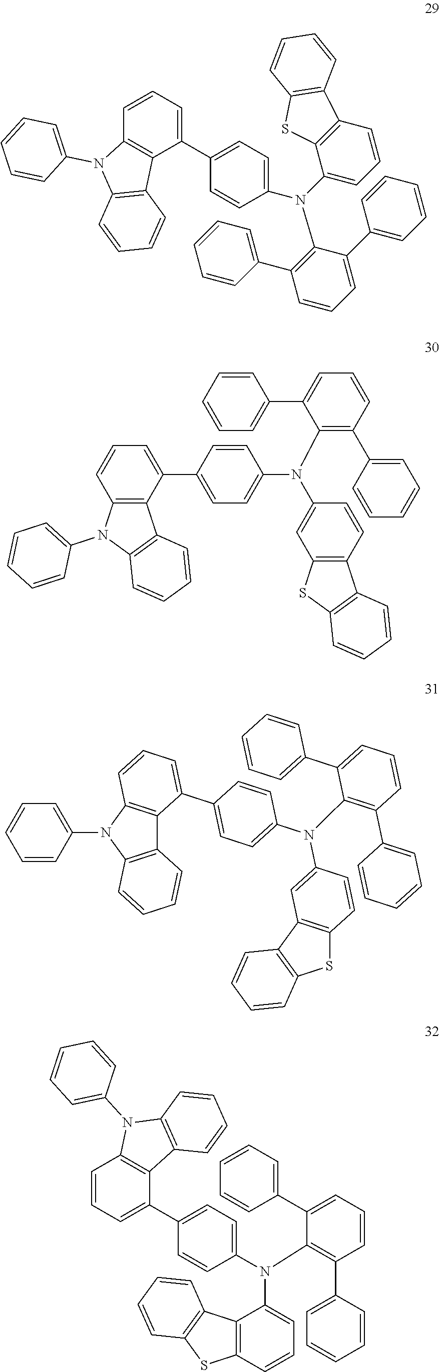 [SMILES: c1ccc(-c2cccc(-c3ccccc3)c2N(c2ccc(-c3cccc4c3c3ccccc3n4-c3ccccc3)cc2)c2cccc3c2sc2ccccc23)cc1 ; c1ccc(-c2cccc(-c3ccccc3)c2N(c2ccc(-c3cccc4c3c3ccccc3n4-c3ccccc3)cc2)c2ccc3c(c2)sc2ccccc23)cc1 ; c1ccc(-c2cccc(-c3ccccc3)c2N(c2ccc(-c3cccc4c3c3ccccc3n4-c3ccccc3)cc2)c2ccc3sc4ccccc4c3c2)cc1 ; c1ccc(-c2cccc(-c3ccccc3)c2N(c2ccc(-c3cccc4c3c3ccccc3n4-c3ccccc3)cc2)c2cccc3sc4ccccc4c23)cc1]

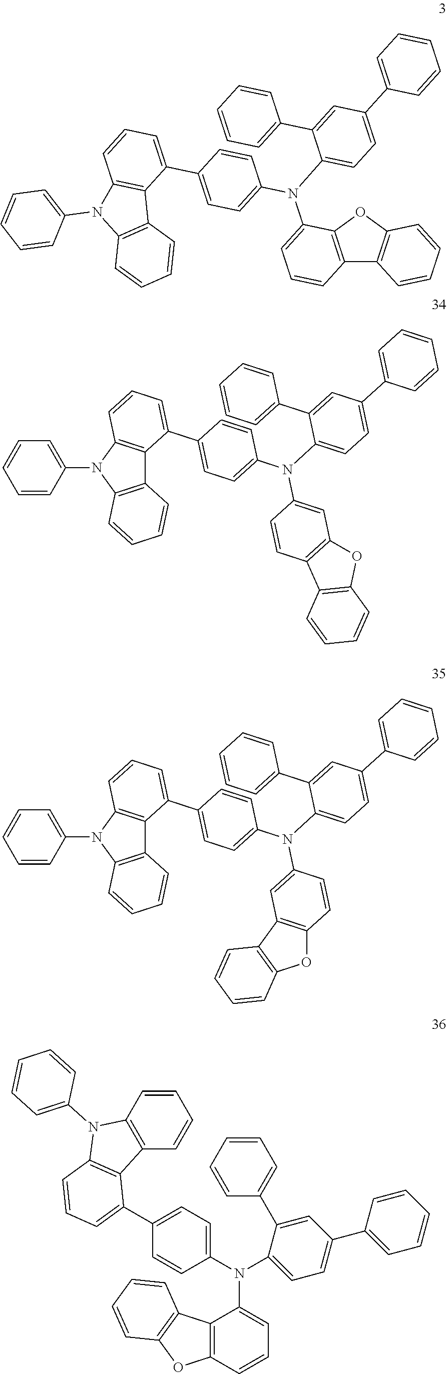

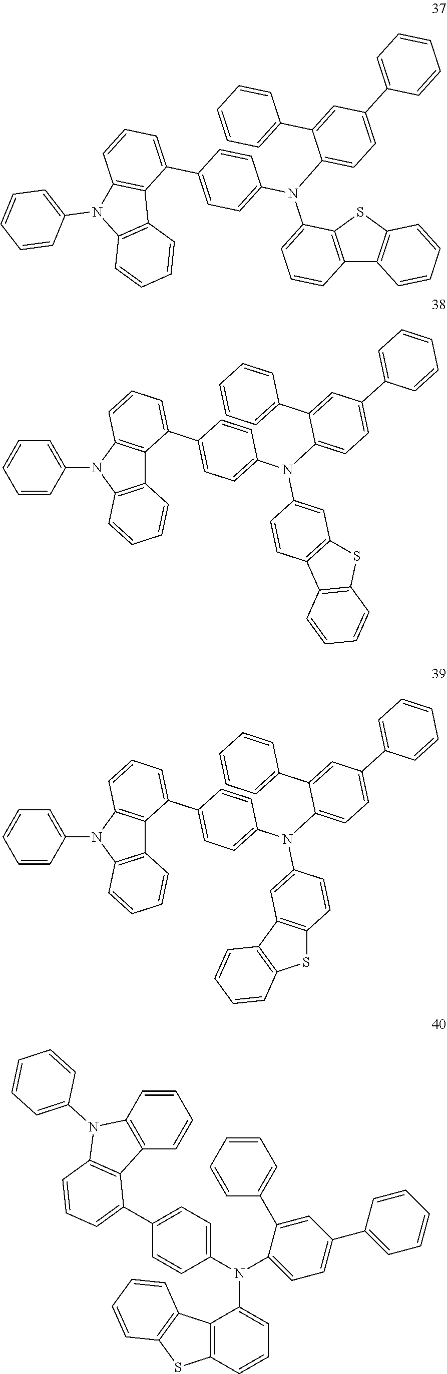

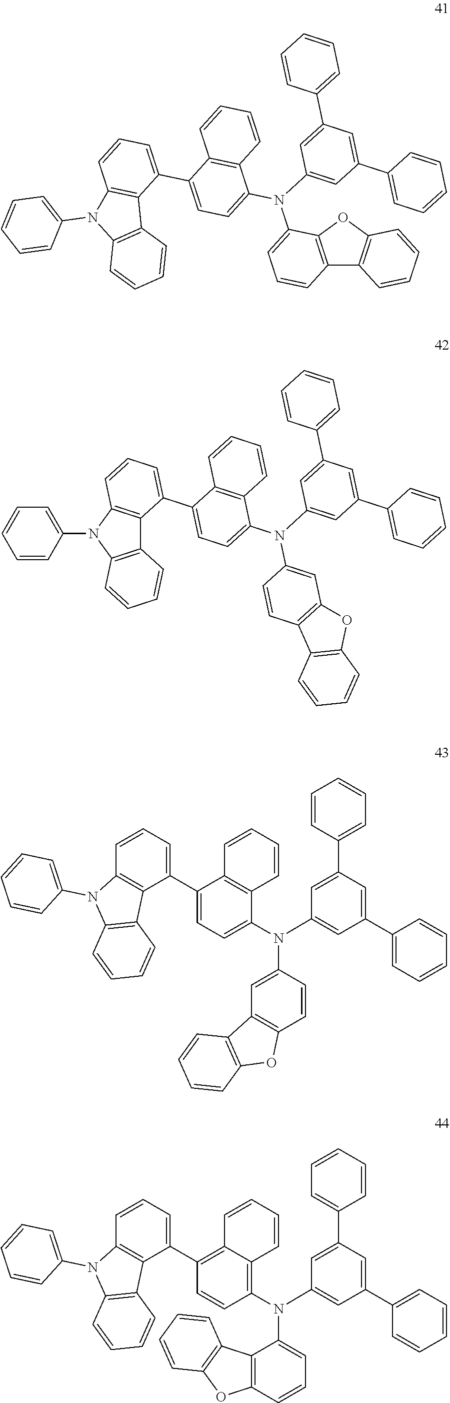 [SMILES: c1ccc(-c2cc(-c3ccccc3)cc(N(c3ccc(-c4cccc5c4c4ccccc4n5-c4ccccc4)c4ccccc34)c3cccc4c3oc3ccccc34)c2)cc1 ; c1ccc(-c2cc(-c3ccccc3)cc(N(c3ccc4c(c3)oc3ccccc34)c3ccc(-c4cccc5c4c4ccccc4n5-c4ccccc4)c4ccccc34)c2)cc1 ; c1ccc(-c2cc(-c3ccccc3)cc(N(c3ccc4oc5ccccc5c4c3)c3ccc(-c4cccc5c4c4ccccc4n5-c4ccccc4)c4ccccc34)c2)cc1 ; c1ccc(-c2cc(-c3ccccc3)cc(N(c3ccc(-c4cccc5c4c4ccccc4n5-c4ccccc4)c4ccccc34)c3cccc4oc5ccccc5c34)c2)cc1]

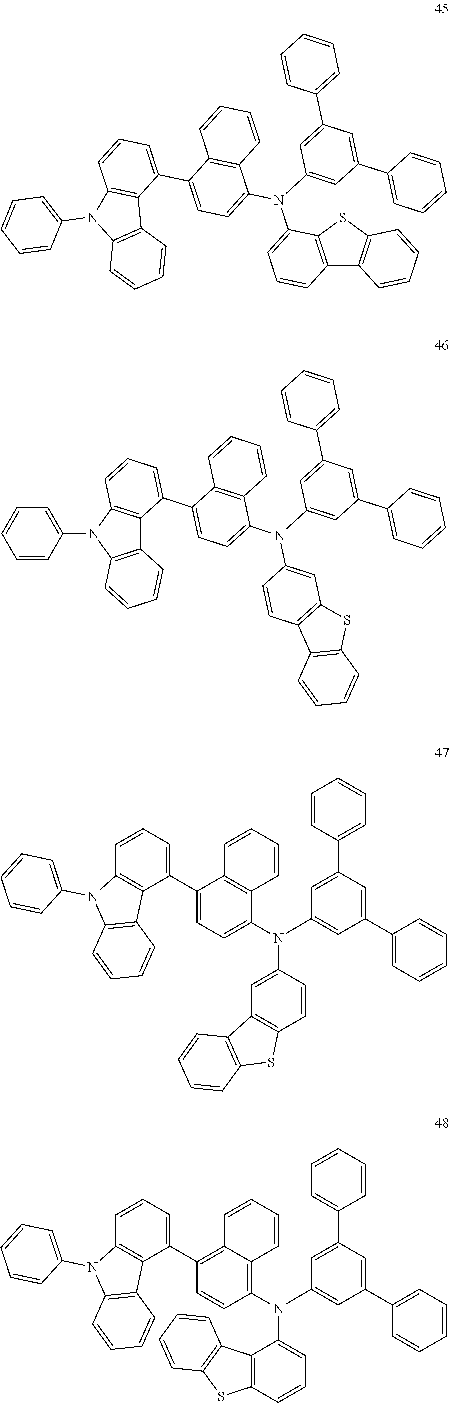 [SMILES: c1ccc(-c2cc(-c3ccccc3)cc(N(c3ccc(-c4cccc5c4c4ccccc4n5-c4ccccc4)c4ccccc34)c3cccc4c3sc3ccccc34)c2)cc1 ; c1ccc(-c2cc(-c3ccccc3)cc(N(c3ccc4c(c3)sc3ccccc34)c3ccc(-c4cccc5c4c4ccccc4n5-c4ccccc4)c4ccccc34)c2)cc1 ; c1ccc(-c2cc(-c3ccccc3)cc(N(c3ccc4sc5ccccc5c4c3)c3ccc(-c4cccc5c4c4ccccc4n5-c4ccccc4)c4ccccc34)c2)cc1 ; c1ccc(-c2cc(-c3ccccc3)cc(N(c3ccc(-c4cccc5c4c4ccccc4n5-c4ccccc4)c4ccccc34)c3cccc4sc5ccccc5c34)c2)cc1]

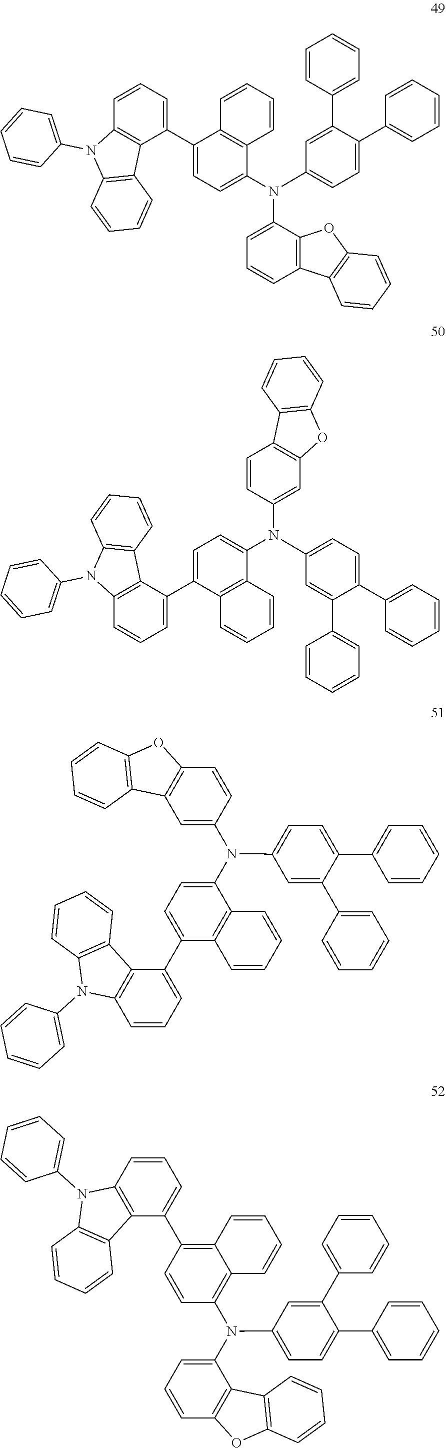 [SMILES: c1ccc(-c2ccc(N(c3ccc(-c4cccc5c4c4ccccc4n5-c4ccccc4)c4ccccc34)c3cccc4c3oc3ccccc34)cc2-c2ccccc2)cc1 ; c1ccc(-c2ccc(N(c3ccc4c(c3)oc3ccccc34)c3ccc(-c4cccc5c4c4ccccc4n5-c4ccccc4)c4ccccc34)cc2-c2ccccc2)cc1 ; c1ccc(-c2ccc(N(c3ccc4oc5ccccc5c4c3)c3ccc(-c4cccc5c4c4ccccc4n5-c4ccccc4)c4ccccc34)cc2-c2ccccc2)cc1 ; c1ccc(-c2ccc(N(c3ccc(-c4cccc5c4c4ccccc4n5-c4ccccc4)c4ccccc34)c3cccc4oc5ccccc5c34)cc2-c2ccccc2)cc1]

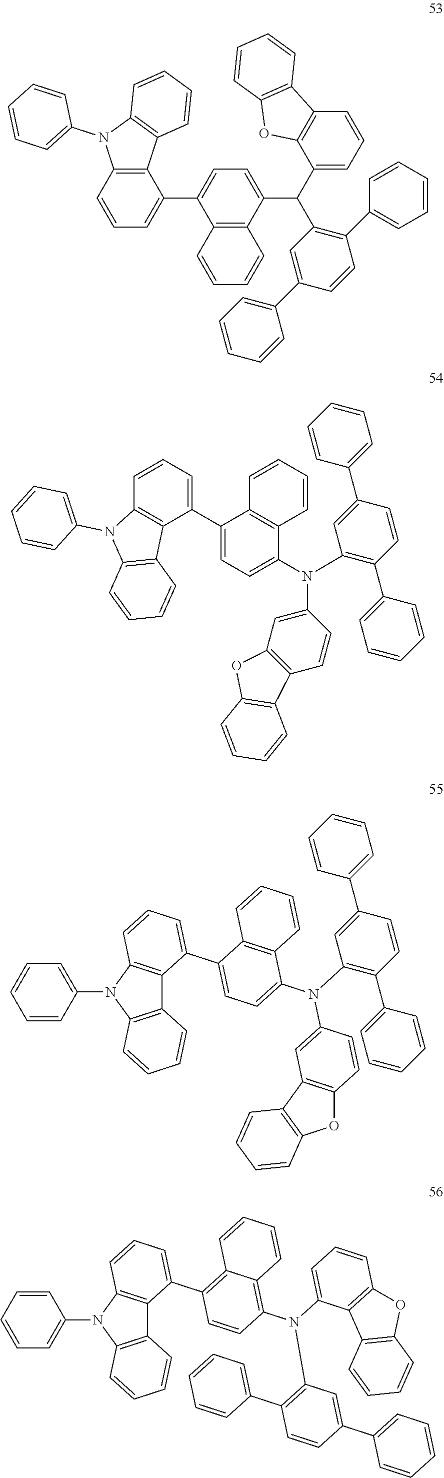 [SMILES: c1ccc(-c2ccc(-c3ccccc3)c(C(c3ccc(-c4cccc5c4c4ccccc4n5-c4ccccc4)c4ccccc34)c3cccc4c3oc3ccccc34)c2)cc1 ; c1ccc(-c2ccc(-c3ccccc3)c(N(c3ccc4c(c3)oc3ccccc34)c3ccc(-c4cccc5c4c4ccccc4n5-c4ccccc4)c4ccccc34)c2)cc1 ; c1ccc(-c2ccc(-c3ccccc3)c(N(c3ccc4oc5ccccc5c4c3)c3ccc(-c4cccc5c4c4ccccc4n5-c4ccccc4)c4ccccc34)c2)cc1 ; c1ccc(-c2ccc(-c3ccccc3)c(N(c3ccc(-c4cccc5c4c4ccccc4n5-c4ccccc4)c4ccccc34)c3cccc4oc5ccccc5c34)c2)cc1]

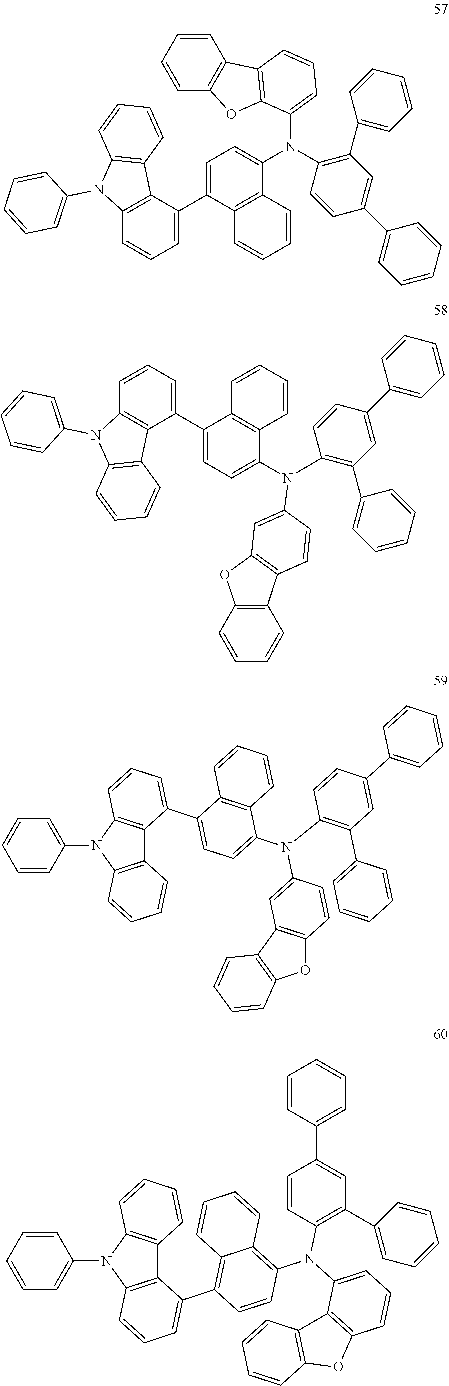 [SMILES: c1ccc(-c2ccc(N(c3ccc(-c4cccc5c4c4ccccc4n5-c4ccccc4)c4ccccc34)c3cccc4c3oc3ccccc34)c(-c3ccccc3)c2)cc1 ; c1ccc(-c2ccc(N(c3ccc4c(c3)oc3ccccc34)c3ccc(-c4cccc5c4c4ccccc4n5-c4ccccc4)c4ccccc34)c(-c3ccccc3)c2)cc1 ; c1ccc(-c2ccc(N(c3ccc4oc5ccccc5c4c3)c3ccc(-c4cccc5c4c4ccccc4n5-c4ccccc4)c4ccccc34)c(-c3ccccc3)c2)cc1 ; c1ccc(-c2ccc(N(c3ccc(-c4cccc5c4c4ccccc4n5-c4ccccc4)c4ccccc34)c3cccc4oc5ccccc5c34)c(-c3ccccc3)c2)cc1]

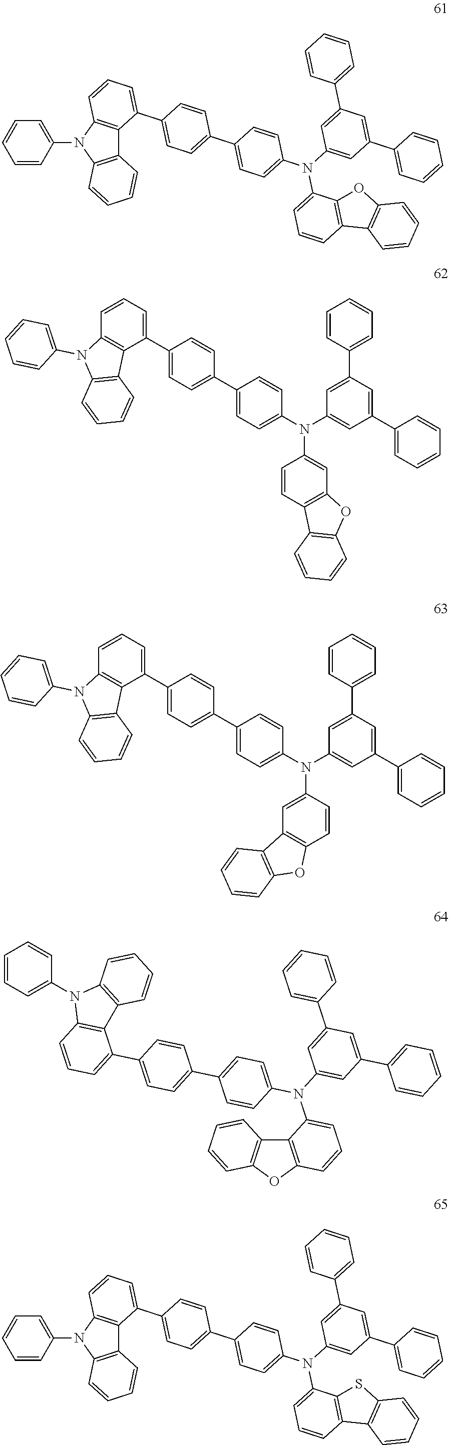

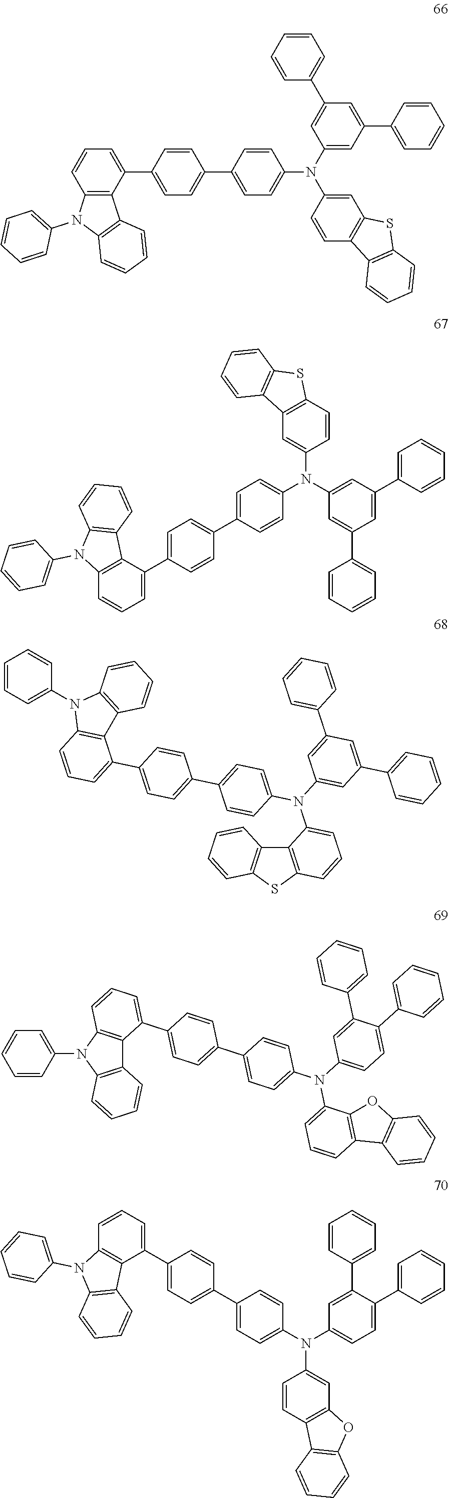

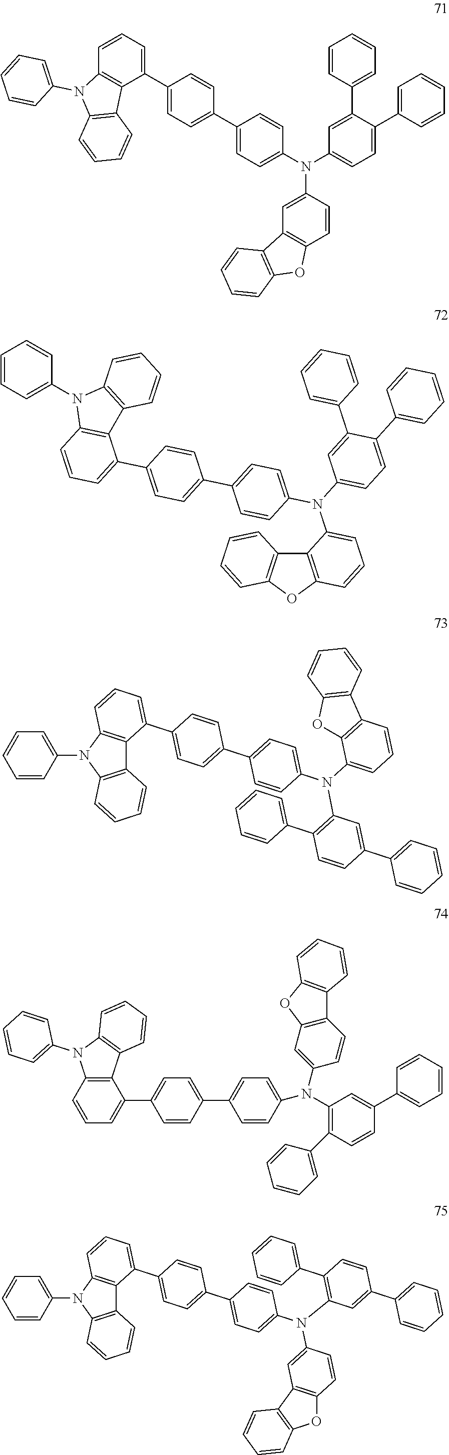

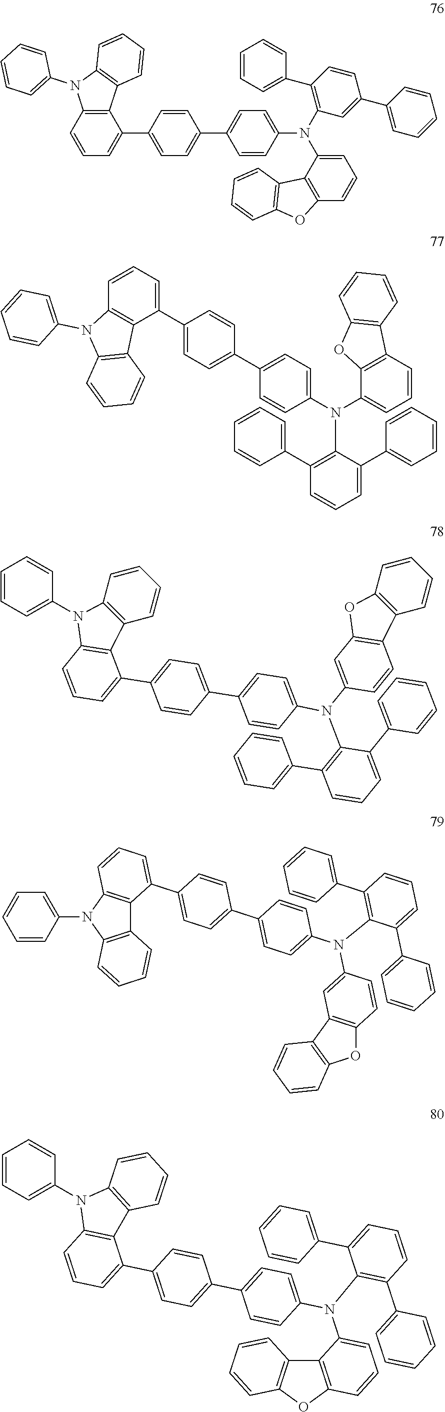

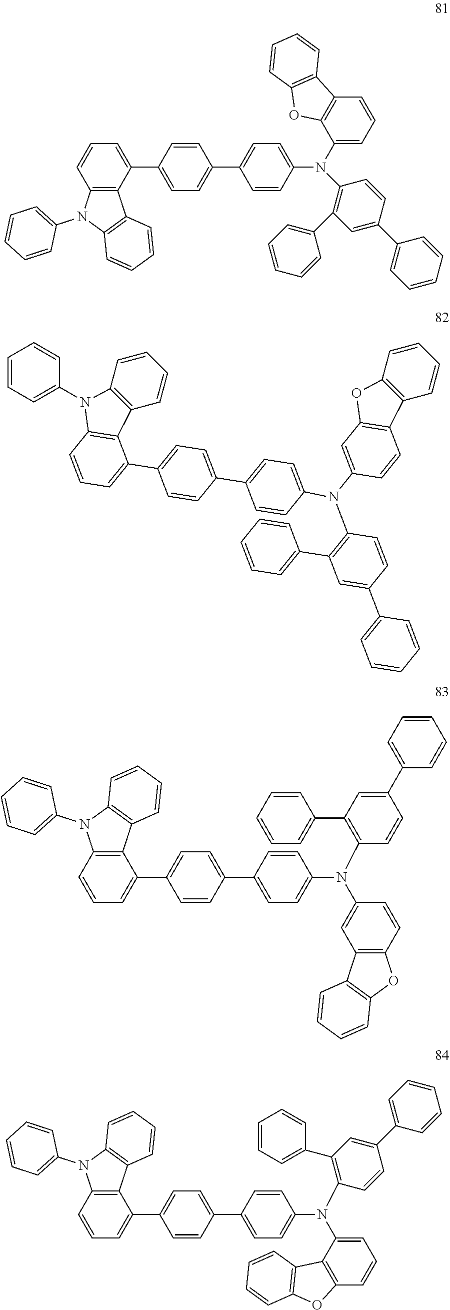 [SMILES: c1ccc(-c2ccc(N(c3ccc(-c4ccc(-c5cccc6c5c5ccccc5n6-c5ccccc5)cc4)cc3)c3cccc4c3oc3ccccc34)c(-c3ccccc3)c2)cc1 ; c1ccc(-c2ccc(N(c3ccc(-c4ccc(-c5cccc6c5c5ccccc5n6-c5ccccc5)cc4)cc3)c3ccc4c(c3)oc3ccccc34)c(-c3ccccc3)c2)cc1 ; c1ccc(-c2ccc(N(c3ccc(-c4ccc(-c5cccc6c5c5ccccc5n6-c5ccccc5)cc4)cc3)c3ccc4oc5ccccc5c4c3)c(-c3ccccc3)c2)cc1 ; c1ccc(-c2ccc(N(c3ccc(-c4ccc(-c5cccc6c5c5ccccc5n6-c5ccccc5)cc4)cc3)c3cccc4oc5ccccc5c34)c(-c3ccccc3)c2)cc1]

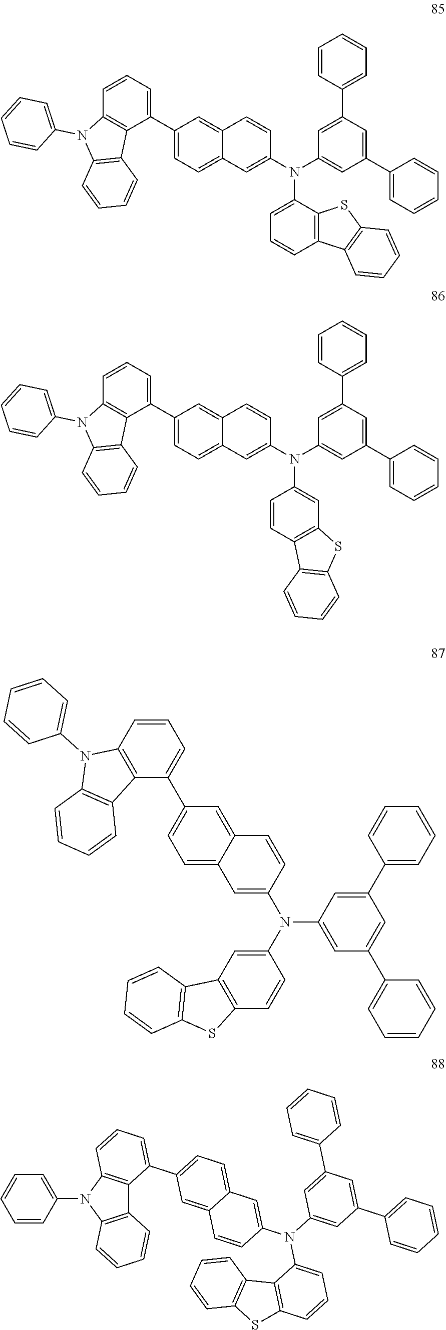 [SMILES: c1ccc(-c2cc(-c3ccccc3)cc(N(c3ccc4cc(-c5cccc6c5c5ccccc5n6-c5ccccc5)ccc4c3)c3cccc4c3sc3ccccc34)c2)cc1 ; c1ccc(-c2cc(-c3ccccc3)cc(N(c3ccc4cc(-c5cccc6c5c5ccccc5n6-c5ccccc5)ccc4c3)c3ccc4c(c3)sc3ccccc34)c2)cc1 ; c1ccc(-c2cc(-c3ccccc3)cc(N(c3ccc4cc(-c5cccc6c5c5ccccc5n6-c5ccccc5)ccc4c3)c3ccc4sc5ccccc5c4c3)c2)cc1 ; c1ccc(-c2cc(-c3ccccc3)cc(N(c3ccc4cc(-c5cccc6c5c5ccccc5n6-c5ccccc5)ccc4c3)c3cccc4sc5ccccc5c34)c2)cc1]

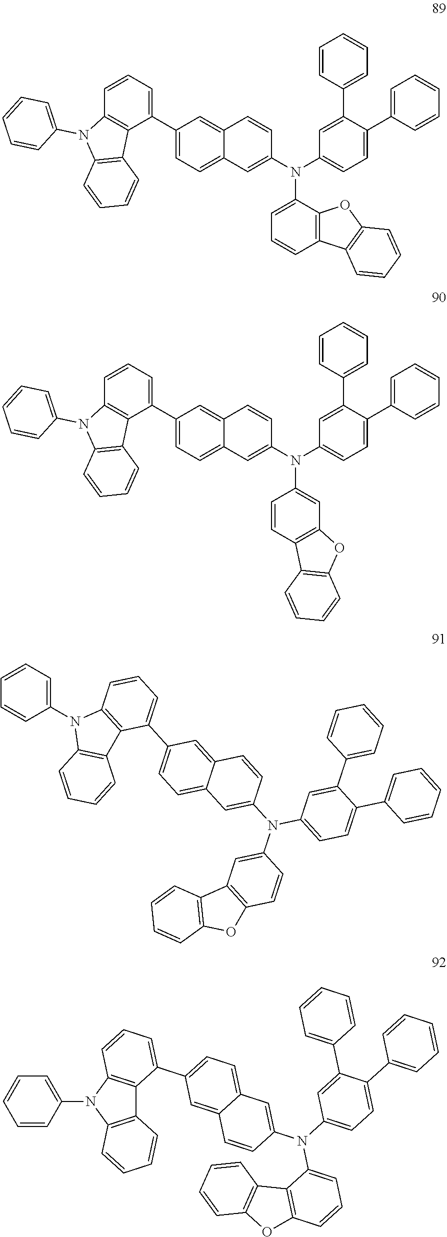 [SMILES: c1ccc(-c2ccc(N(c3ccc4cc(-c5cccc6c5c5ccccc5n6-c5ccccc5)ccc4c3)c3cccc4c3oc3ccccc34)cc2-c2ccccc2)cc1 ; c1ccc(-c2ccc(N(c3ccc4cc(-c5cccc6c5c5ccccc5n6-c5ccccc5)ccc4c3)c3ccc4c(c3)oc3ccccc34)cc2-c2ccccc2)cc1 ; c1ccc(-c2ccc(N(c3ccc4cc(-c5cccc6c5c5ccccc5n6-c5ccccc5)ccc4c3)c3ccc4oc5ccccc5c4c3)cc2-c2ccccc2)cc1 ; c1ccc(-c2ccc(N(c3ccc4cc(-c5cccc6c5c5ccccc5n6-c5ccccc5)ccc4c3)c3cccc4oc5ccccc5c34)cc2-c2ccccc2)cc1]

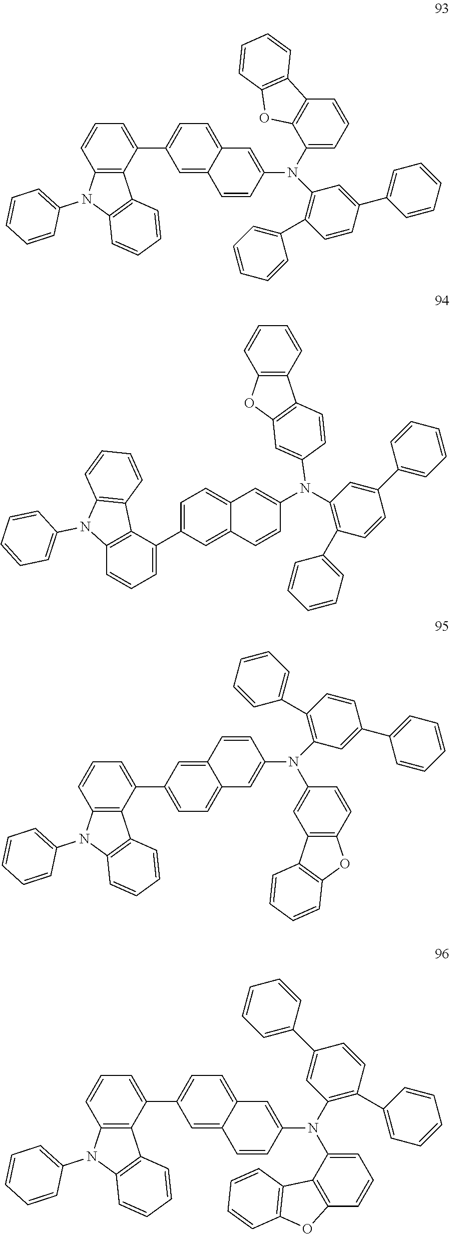 [SMILES: c1ccc(-c2ccc(-c3ccccc3)c(N(c3ccc4cc(-c5cccc6c5c5ccccc5n6-c5ccccc5)ccc4c3)c3cccc4c3oc3ccccc34)c2)cc1 ; c1ccc(-c2ccc(-c3ccccc3)c(N(c3ccc4cc(-c5cccc6c5c5ccccc5n6-c5ccccc5)ccc4c3)c3ccc4c(c3)oc3ccccc34)c2)cc1 ; c1ccc(-c2ccc(-c3ccccc3)c(N(c3ccc4cc(-c5cccc6c5c5ccccc5n6-c5ccccc5)ccc4c3)c3ccc4oc5ccccc5c4c3)c2)cc1 ; c1ccc(-c2ccc(-c3ccccc3)c(N(c3ccc4cc(-c5cccc6c5c5ccccc5n6-c5ccccc5)ccc4c3)c3cccc4oc5ccccc5c34)c2)cc1]

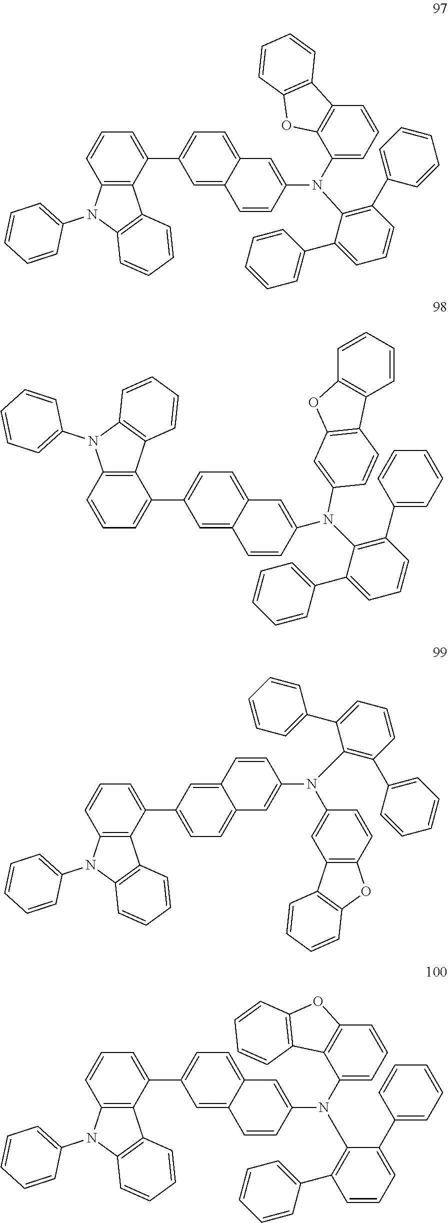 [SMILES: c1ccc(-c2cccc(-c3ccccc3)c2N(c2ccc3cc(-c4cccc5c4c4ccccc4n5-c4ccccc4)ccc3c2)c2cccc3c2oc2ccccc23)cc1 ; c1ccc(-c2cccc(-c3ccccc3)c2N(c2ccc3cc(-c4cccc5c4c4ccccc4n5-c4ccccc4)ccc3c2)c2ccc3c(c2)oc2ccccc23)cc1 ; c1ccc(-c2cccc(-c3ccccc3)c2N(c2ccc3cc(-c4cccc5c4c4ccccc4n5-c4ccccc4)ccc3c2)c2ccc3oc4ccccc4c3c2)cc1 ; c1ccc(-c2cccc(-c3ccccc3)c2N(c2ccc3cc(-c4cccc5c4c4ccccc4n5-c4ccccc4)ccc3c2)c2cccc3oc4ccccc4c23)cc1]

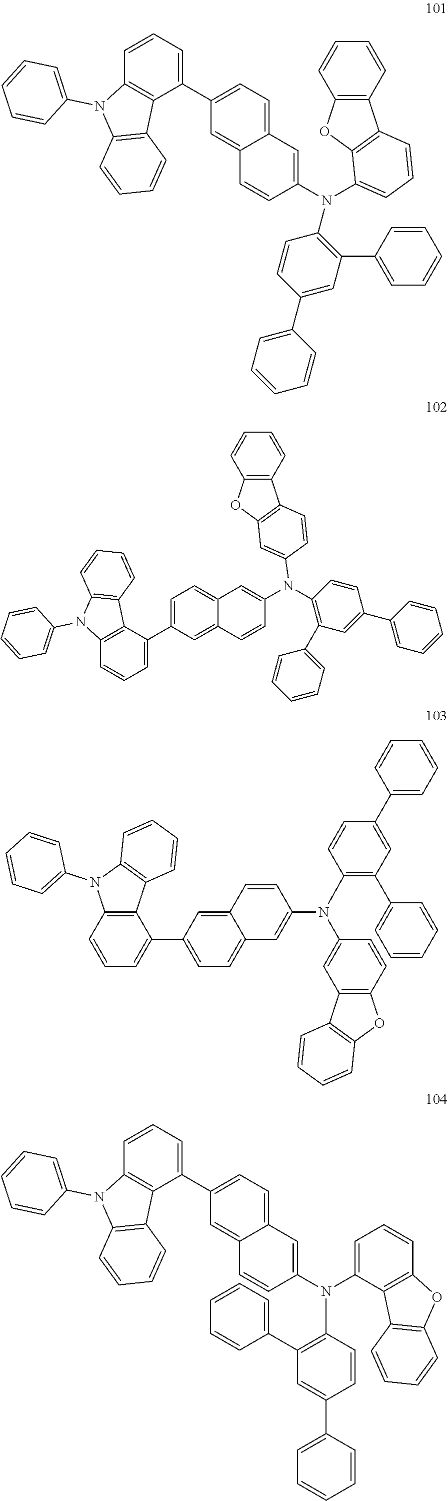

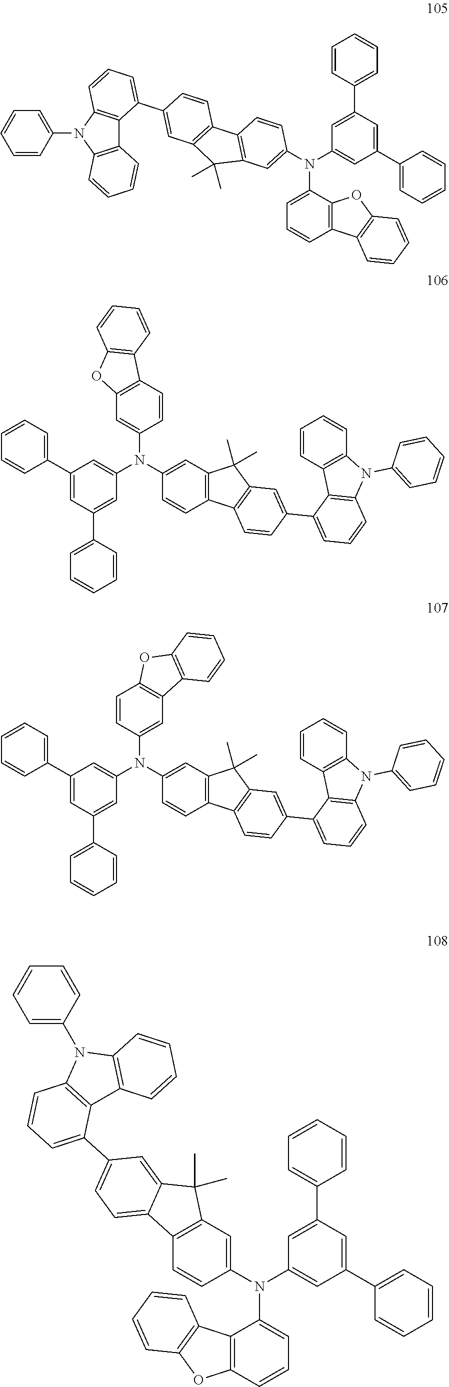 [SMILES: CC1(C)c2cc(-c3cccc4c3c3ccccc3n4-c3ccccc3)ccc2-c2ccc(N(c3cc(-c4ccccc4)cc(-c4ccccc4)c3)c3cccc4c3oc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4c(c3)oc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4oc5ccccc5c4c3)cc21 ; CC1(C)c2cc(-c3cccc4c3c3ccccc3n4-c3ccccc3)ccc2-c2ccc(N(c3cc(-c4ccccc4)cc(-c4ccccc4)c3)c3cccc4oc5ccccc5c34)cc21]

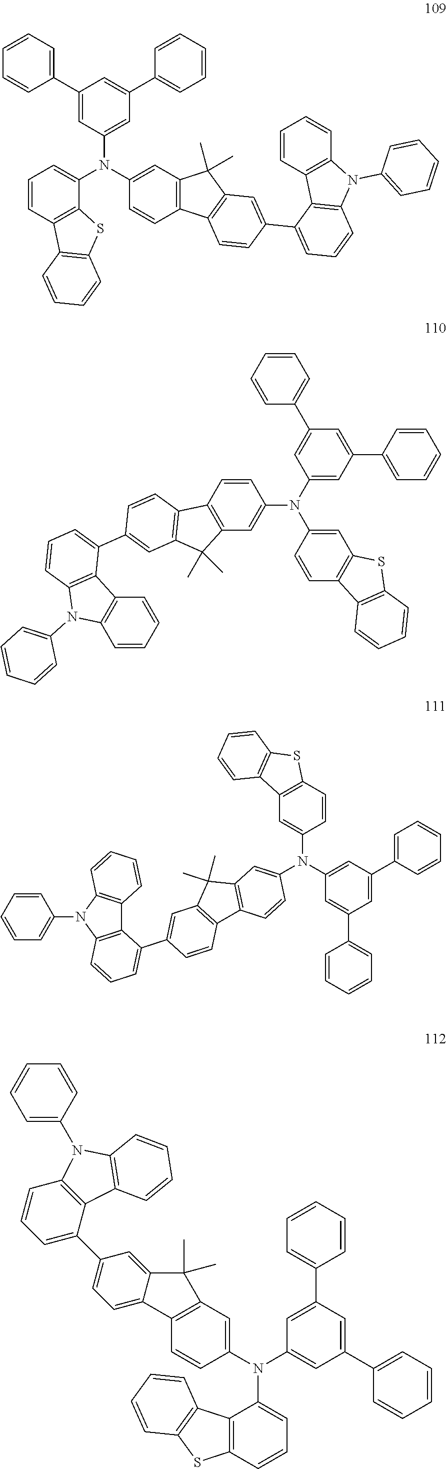 [SMILES: CC1(C)c2cc(-c3cccc4c3c3ccccc3n4-c3ccccc3)ccc2-c2ccc(N(c3cc(-c4ccccc4)cc(-c4ccccc4)c3)c3cccc4c3sc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4c(c3)sc3ccccc34)cc21 ; CC1(C)c2cc(-c3cccc4c3c3ccccc3n4-c3ccccc3)ccc2-c2ccc(N(c3cc(-c4ccccc4)cc(-c4ccccc4)c3)c3ccc4sc5ccccc5c4c3)cc21 ; CC1(C)c2cc(-c3cccc4c3c3ccccc3n4-c3ccccc3)ccc2-c2ccc(N(c3cc(-c4ccccc4)cc(-c4ccccc4)c3)c3cccc4sc5ccccc5c34)cc21]

3. An organic light emitting device including a first electrode; a second electrode; and one or more organic layers disposed between the first electrode and the second electrode, wherein one or more of the organic layers include an organic compound represented by Formula I according to claim 1.

4. The organic light emitting device of claim 3, wherein:
the organic layers include one or more layers selected from a hole injecting layer, a hole transport layer, a layer having functions of both hole injection and hole transport, an electron transport layer, an electron injecting layer, a layer having functions of both electron transport and electron injection, an electron blocking layer, a hole blocking layer, and a light emitting layer, and
the one or more layers include an organic light emitting compound represented by the Formula I.

5. The organic light emitting device of claim 4, wherein the electron blocking layer includes an organic light emitting compound represented by the Formula I.
